SEQUENCE LISTING (45 sequences)

SEQ ID NO: 1 - DNA, 3325 bp, Arabidopsis thaliana
aaaagacttatttctcaaaaaaaatggcgagttataatcttcgttcacaagttcgtcttatagcaataacaatagtaatcatcattactctctcaactccgatcacaaccaacacatcaccacaaccatggaatatcctttcacacaacgaattcgccggaaaactcacctcctcctcctcctccgtcgaatcagccgccacagatttcggccacgtcaccaaaatcttcccttccgccgtcttaatcccttcctccgttgaagacatcacagatctcataaaactctcttttgactctcaactgtcttttcctttagccgctcgtggtcacggacacagccaccgtggccaagcctcggctaaagacggagttgtggtcaacatgcggtccatggtaaaccgggatcgaggtatcaaggtgtctaggacctgtttatatgttgacgtggacgctgcgtggctatggattgaggtgttgaataaaactttggagttagggttaacgccggtttcttggacggattatttgtatttaacagtcggtgggacgttatcaaacggcggaattagtggacaaacgtttcggtacggtccacagatcactaatgttctagagatggatgttattactggtacgtaccacgatctttttcacacagagattaaaaaaaacagtaatagtgattttaacttcgtacgtttctgatagacaacaaagaacttcgtacgtttttcgaagttttttcgtctttttcattttagatctgcgcggccatttttggttatgctattgtttgtttgtattgtttgtctctgtttatttatttctcgaacttgttgatagcttttcttcttttcacacatcaatctaatcaccttttttggtcttaagattagaaagaagatacggactaggtaaaaataggtggttgtaaacgtagacgcattaaaaaaatattggtttttttattttttgataagcaaaattggtggttggtctaagattataaacttgatattaatgcaaaggtcgatctagcaatagaagattaatcaatattcttggtgttttaacaacagattatttcatcattaaaatcgtgaaacaaagaaattttggtagtatacattacgtgtagttttgttagtttattaaaaaaaatagtatatagttttgttaaaacgcgatttatttagtaacacattagtatattacacgtttaaccaactaaacttttttttttgaataattatgttctatatttcttactcaaattatgcaaatttcgtggattcgaagtcaaatttctgcgaaatttacatggtcatatattataaaactgttcatataacccggtgaacaaacagacaattaagggtttgaatggttacggcggttggggcggacacaaccgtcaatagatcagaccgttttttatttaccattcatcaattatattccgcagtggtttggggtaaaaaaaatagaagaaaaccgcagcggaccaattccataccgtttttacatacaaataaacatggtgcgcaacggtttattgtccgcctcaaaaatgaaatggactaaaccgcagataaattagaccgctttgtccgctgcctccattcatagactaaaaaaaaacaaccaaaaaaaaaatggtcccacgcccatgattttacacgaggtttcttgtggcgtaaggacaaaactcaaaagttcataacgtttggtcctaaccaggtgtaatggattaagtaacagtcaattttcttattatagctgtatccattatgtccacatatgcatccatatacattacactgttggtctcaagtgtagttagattacgaagactttcaagttccattttttggttaggagataaacataatttaatgataccgactttagcactctaggctcaaaacaagtacagaagagaatagttttatttcaaactcgttgcattgttgtatcaattaattgtgttagtctttgtatattcttacataacggtccaagtttgttgaaatagtttacttactaaacttttcctaatggggtcaaattttattttataggaaaaggagagattgcaacttgttccaaggacatgaactcggatcttttcttcgcggtgttaggaggtttgggtcaattcggcattataacaagagccagaattaaacttgaagtagctccgaaaagggtatgttaaatttgtaaattatgcaactacagaaaattctatgaaatttatgaatgaacatatatgcatttttggatttttgtaggccaagtggttaaggtttctatacatagatttctccgaattcacaagagatcaagaacgagtgatatcgaaaacggacggtgtagatttcttagaaggttccattatggtggaccatggcccaccggataactggagatccacgtattatccaccgtccgatcacttgaggatcgcctcaatggtcaaacgacatcgtgtcatctactgccttgaagtcgtcaagtattacgacgaaacttctcaatacacagtcaacgaggtccgtacatacatacaatcataaatcatacatgtataattgggagatctttatgcattattcaattatattaatttactttagttatttaacttatgcaggaaatggaggagttaagcgatagtttaaaccatgtaagagggtttatgtacgagaaagatgtgacgtatatggatttcctaaaccgagttcgaaccggagagctaaacctgaaatccaaaggccaatgggatgttccacatccatggcttaatctcttcgtaccaaaaactcaaatctccaaatttgatgatggtgtttttaagggtattatcctaagaaataacatcactagcggtcctgttcttgtttatcctatgaatcgcaacaagtaagtttaactcgatattgcaaaatttactatctacattttcgttttggaatccgaaatattcttacaagctaattttatgcggcgtttttaggtggaatgatcggatgtctgccgctatacccgaggaagatgtattttatgcggtagggtttttaagatccgcgggttttgacaattgggaggcttttgatcaagaaaacatggaaatactgaagttttgtgaggatgctaatatgggggttatacaatatcttccttatcattcatcacaagaaggatgggttagacattttggtccgaggtggaatattttcgtagagagaaaatataaatatgatcccaaaatgatattatcaccgggacaaaatatatttcaaaaaataaactcgagttag

SEQ ID NO: 2 - DNA, 1572 bp, Arabidopsis thaliana
atggcgagttataatcttcgttcacaagttcgtcttatagcaataacaatagtaatcatcattactctctcaactccgatcacaaccaacacatcaccacaaccatggaatatcctttcacacaacgaattcgccggaaaactcacctcctcctcctcctccgtcgaatcagccgccacagatttcggccacgtcaccaaaatcttcccttccgccgtcttaatcccttcctccgttgaagacatcacagatctcataaaactctcttttgactctcaactgtcttttcctttagccgctcgtggtcacggacacagccaccgtggccaagcctcggctaaagacggagttgtggtcaacatgcggtccatggtaaaccgggatcgaggtatcaaggtgtctaggacctgtttatatgttgacgtggacgctgcgtggctatggattgaggtgttgaataaaactttggagttagggttaacgccggtttcttggacggattatttgtatttaacagtcggtgggacgttatcaaacggcggaattagtggacaaacgtttcggtacggtccacagatcactaatgttctagagatggatgttattactggaaaaggagagattgcaacttgttccaaggacatgaactcggatcttttcttcgcggtgttaggaggtttgggtcaattcggcattataacaagagccagaattaaacttgaagtagctccgaaaagggccaagtggttaaggtttctatacatagatttctccgaattcacaagagatcaagaacgagtgatatcgaaaacggacggtgtagatttcttagaaggttccattatggtggaccatggcccaccggataactggagatccacgtattatccaccgtccgatcacttgaggatcgcctcaatggtcaaacgacatcgtgtcatctactgccttgaagtcgtcaagtattacgacgaaacttctcaatacacagtcaacgaggaaatggaggagttaagcgatagtttaaaccatgtaagagggtttatgtacgagaaagatgtgacgtatatggatttcctaaaccgagttcgaaccggagagctaaacctgaaatccaaaggccaatgggatgttccacatccatggcttaatctcttcgtaccaaaaactcaaatctccaaatttgatgatggtgtttttaagggtattatcctaagaaataacatcactagcggtcctgttcttgtttatcctatgaatcgcaacaagtggaatgatcggatgtctgccgctatacccgaggaagatgtattttatgcggtagggtttttaagatccgcgggttttgacaattgggaggcttttgatcaagaaaacatggaaatactgaagttttgtgaggatgctaatatgggggttatacaatatcttccttatcattcatcacaagaaggatgggttagacattttggtccgaggtggaatattttcgtagagagaaaatataaatatgatcccaaaatgatattatcaccgggacaaaatatatttcaaaaaataaactcgagttag

SEQ ID NO: 3 - protein, 523 aa, Arabidopsis thaliana
MASYNLRSQVRLIAITIVIIITLSTPITTNTSPQPWNILSHNEFAGKLTSSSSSVESAATDFGHVTKIFPSAVLIPSSVEDITDLIKLSFDSQLSFPLAARGHGHSHRGQASAKDGVVVNMRSMVNRDRGIKVSRTCLYVDVDAAWLWIEVLNKTLELGLTPVSWTDYLYLTVGGTLSNGGISGQTFRYGPQITNVLEMDVITGKGEIATCSKDMNSDLFFAVLGGLGQFGIITRARIKLEVAPKRAKWLRFLYIDFSEFTRDQERVISKTDGVDFLEGSIMVDHGPPDNWRSTYYPPSDHLRIASMVKRHRVIYCLEVVKYYDETSQYTVNEEMEELSDSLNHVRGFMYEKDVTYMDFLNRVRTGELNLKSKGQWDVPHPWLNLFVPKTQISKFDDGVFKGIILRNNITSGPVLVYPMNRNKWNDRMSAAIPEEDVFYAVGFLRSAGFDNWEAFDQENMEILKFCEDANMGVIQYLPYHSSQEGWVRHFGPRWNIFVERKYKYDPKMILSPGQNIFQKINSS

SEQ ID NO: 4 - DNA, 3905 bp, Arabidopsis thaliana
ataaacgcacaaactcgttaaatttgtacgaatataattttttttaaaacactcgttataatatattaaagtttcacccaaaccgaaaaaagagagaatctgtgcatgttgctcagaaaatcttcaaagcgtaatctgggcttacgttagctctcacgaacccccaaggatcttctatatatgttttttcatttccccataaaatctttcattatctaaaaaatattattatcgtatcttttttcttctatatattcttcctcctcaatcttgattcttgtttcttgagtattctttgatgaatcgtgaaatgacgtcaagctttcttctcctgacgttcgccatatgtaaactgatcatagccgtgggtctaaacgtgggccccagtgagctcctccgcatcggagccatagatgtcgacggccacttcaccgtccacccttccgacttagcctccgtctcctcagacttcggtatgctgaagtcacctgaagagccattggccgtgcttcatccatcatcggccgaagacgtggcacgactcgtcagaacagcttacggttcagccacggcgtttccggtctcagcccgaggccacggccattccataaacggacaagccgcggcggggaggaacggtgtggtggttgaaatgaaccacggcgtaaccgggacgcccaagccactcgtccgaccggatgaaatgtatgtggatgtatggggtggagagttatgggtcgatgtgttgaagaaaacgttggagcatggcttagcaccaaaatcatggacggattacttgtatctaaccgttggaggtacactctccaatgcaggaatcagtggtcaagcttttcaccatggtcctcaaattagtaacgtccttgagctcgacgttgtaactggttagtattaaaacattcaagttcatatattttaaatgcttttgtctgaagttttactaataacaagaaattgataccaaaaagtagggaaaggagaggtgatgagatgctcagaagaagagaacacaaggctattccatggagttcttggtggattaggtcaatttgggatcatcactcgagcacgaatctctctcgaaccagctccccaaagggtaatatttttttaatgactagctatcaaaaatccctggcgggtccatacgttgtaatctttttagtttttactgttgatggtattttttatatattttggataataaaaccctaaaatggtatattgtgatgacaggtgagatggatacgggtattgtattcgagcttcaaagtgtttacggaggaccaagagtacttaatctcaatgcatggtcaattaaagtttgattacgtggaaggttttgtgattgtggacgaaggactcgtcaacaattggagatcttctttcttctctccacgtaaccccgtcaagatctcctctgttagttccaacggctctgttttgtattgccttgagatcaccaagaactaccacgactccgactccgaaatcgttgatcaggtcactttcattattcacttagaaaaaagcgatattttcattttttatattgatgaatatctggaaggatttaacgctatgcgactattgggaaatcattatgaaaaaatatttagtttatatgattgaaagtggtctccatagtatttttgttgtgtcgactttattataacttaaatttggaagaggacatgaagaagaagccagagaggatctacagagatctagcttttccacctgaacttaataatgcacatttatataattatttttcttcttctaaagtttagtttatcactagcgaattaatcatggttactaattaagtagtggacagggtcatggaccactcactcaccaaataatgattcctctttactcttaagtttaattttaataaaaccaactctactggaatcttaacttatccttggttttggtaggcttttatagcaacacggtttttttaattttcctattccagattttgtatattaaatgtcgattttttttctttttgtttcaggaagttgagattctgatgaagaaattgaatttcataccgacatcggtctttacaacggatttacaatatgtggactttctcgaccgggtacacaaggccgaattgaagctccggtccaagaatttatgggaggttccacacccatggctcaacctcttcgtgccaaaatcaagaatctctgacttcgataaaggcgttttcaagggcattttgggaaataaaacaagtggccctattcttatctaccccatgaacaaagacaagtaagtcttgacattaccattgattactacttctaaatttcttctctagaaaaaagaataaaacgagttttgcattgcatgcatgcaaagttacacttgtggggattaattagtggtccaagaaaaaaagtttgtcaaaattgaaaaaaactagacacgtggtacatgggattgtccgaaaaacgttgtccacatgtgcatcgaaccagctaagattgacaacaacacttcgtcggctcgtatttctctttttgttttgtgaccaaatccgatggtccagattgggtttatttgtttttaagttcctagaactcatggtgggtgggtcccaatcagattctcctagaccaaaccgatctcaacgaaccctccgcacatcattgattattacattaatatagatattgtcgttgctgacgtgtcgtaatttgatgttattgtcagatgggacgagaggagctcagccgtgacgccggatgaggaagttttctatctggtggctctattgagatcagctttaacggacggtgaagagacacagaagctagagtatctgaaagatcagaaccgtcggatcttggagttctgtgaacaagccaagatcaatgtgaagcagtatcttcctcaccacgcaacacaggaagagtgggtggctcattttggggacaagtgggatcggttcagaagcttaaaggctgagtttgatccgcgacacatactcgctactggtcagagaatctttcaaaacccatctttgtctttgtttcctccgtcgtcgtcttcttcgtcagcggcttcatggtgacacaggacaaaaccaagaggaaagtagtagttgagaaatattcttcctgatgtatatatatgacctaatagagaagcatcagagagattaggttgggtcttttgttagtaggggttttaaaattagagtagaagattcaaaattagtcaaatttattgtgttgtttttatacatcacggattttggaaatgggtttgggacagcttaaacccaattaaattaaaaatagacaacaaacctcaaagactgttttggactattttacattttttttggttgatcgaggtttatactttatcgtcgtggaaccaccatcgggtctcctttggagatcttccgttcttacaattcttcacatatcggtcattatcggttggtcgttgctgcaacaaaacaagccaagcgagacgaaggttcagcttcagcaacaaagagatcaaagtttatatattaatggtgagtgtaagagctgtaagtaccttttcagtcgagcttgagagcatggagaggatgctaatgcagatagaactcaccgtcatggctggagaccacgaatcatacaaaatatctgcaacgatgatacacaacatcagatacttcttccaggaggcctatgttaaataataagctactgaaatttgagtgtacccttggatctaaaagcaaaatctcaagcaatatgagtttcgaagagggtcttatcttcgggtaagacttcaaatgagtttaggtatacaaacgacttatgttacctctcacattgtgattattaatatattagagatcatac

SEQ ID NO: 5 - DNA, 1623 bp, Arabidopsis thaliana
atgaatcgtgaaatgacgtcaagctttcttctcctgacgttcgccatatgtaaactgatcatagccgtgggtctaaacgtgggccccagtgagctcctccgcatcggagccatagatgtcgacggccacttcaccgtccacccttccgacttagcctccgtctcctcagacttcggtatgctgaagtcacctgaagagccattggccgtgcttcatccatcatcggccgaagacgtggcacgactcgtcagaacagcttacggttcagccacggcgtttccggtctcagcccgaggccacggccattccataaacggacaagccgcggcggggaggaacggtgtggtggttgaaatgaaccacggcgtaaccgggacgcccaagccactcgtccgaccggatgaaatgtatgtggatgtatggggtggagagttatgggtcgatgtgttgaagaaaacgttggagcatggcttagcaccaaaatcatggacggattacttgtatctaaccgttggaggtacactctccaatgcaggaatcagtggtcaagcttttcaccatggtcctcaaattagtaacgtccttgagctcgacgttgtaactgggaaaggagaggtgatgagatgctcagaagaagagaacacaaggctattccatggagttcttggtggattaggtcaatttgggatcatcactcgagcacgaatctctctcgaaccagctccccaaagggtgagatggatacgggtattgtattcgagcttcaaagtgtttacggaggaccaagagtacttaatctcaatgcatggtcaattaaagtttgattacgtggaaggttttgtgattgtggacgaaggactcgtcaacaattggagatcttctttcttctctccacgtaaccccgtcaagatctcctctgttagttccaacggctctgttttgtattgccttgagatcaccaagaactaccacgactccgactccgaaatcgttgatcaggaagttgagattctgatgaagaaattgaatttcataccgacatcggtctttacaacggatttacaatatgtggactttctcgaccgggtacacaaggccgaattgaagctccggtccaagaatttatgggaggttccacacccatggctcaacctcttcgtgccaaaatcaagaatctctgacttcgataaaggcgttttcaagggcattttgggaaataaaacaagtggccctattcttatctaccccatgaacaaagacaaatgggacgagaggagctcagccgtgacgccggatgaggaagttttctatctggtggctctattgagatcagctttaacggacggtgaagagacacagaagctagagtatctgaaagatcagaaccgtcggatcttggagttctgtgaacaagccaagatcaatgtgaagcagtatcttcctcaccacgcaacacaggaagagtgggtggctcattttggggacaagtgggatcggttcagaagcttaaaggctgagtttgatccgcgacacatactcgctactggtcagagaatctttcaaaacccatctttgtctttgtttcctccgtcgtcgtcttcttcgtcagcggcttcatggtga

SEQ ID NO: 6 - protein, 540 aa, Arabidopsis thaliana
MNREMTSSFLLLTFAICKLIIAVGLNVGPSELLRIGAIDVDGHFTVHPSDLASVSSDFGMLKSPEEPLAVLHPSSAEDVARLVRTAYGSATAFPVSARGHGHSINGQAAAGRNGVVVEMNHGVTGTPKPLVRPDEMYVDVWGGELWVDVLKKTLEHGLAPKSWTDYLYLTVGGTLSNAGISGQAFHHGPQISNVLELDVVTGKGEVMRCSEEENTRLFHGVLGGLGQFGIITRARISLEPAPQRVRWIRVLYSSFKVFTEDQEYLISMHGQLKFDYVEGFVIVDEGLVNNWRSSFFSPRNPVKISSVSSNGSVLYCLEITKNYHDSDSEIVDQEVEILMKKLNFIPTSVFTTDLQYVDFLDRVHKAELKLRSKNLWEVPHPWLNLFVPKSRISDFDKGVFKGILGNKTSGPILIYPMNKDKWDERSSAVTPDEEVFYLVALLRSALTDGEETQKLEYLKDQNRRILEFCEQAKINVKQYLPHHATQEEWVAHFGDKWDRFRSLKAEFDPRHILATGQRIFQNPSLSLFPPSSSSSSAASW

SEQ ID NO: 7 - DNA, 2796 bp, Brassica napus
  exon (1)..(613): exon1
  exon (1546)..(1673): exon2
  exon (1780)..(2040): exon3
  exon (2154)..(2422): exon4
  variation (2244)..(2244): C to T substitution in YIIN501
  exon (2508)..(2796): exon5
atggtgagctataattttccatcacaaattcatcttctaatgataacgatattagttatcatcacaactcttttaactccgatcacaaccaacacatcatcactaccgtggaatatcctttccaatgacaacttcgccggaaaactcactaccgcatcatcctccgtcgaatcagctgccactgatttcggccacatcaccaaaatctttccctccgccgtcttaaacccttcctccgtccaagacatcaccgatcttataaaactttctttcgattctcaatcctcttttcctctagccgctcgtggccacggacacagccaccgtggtcaagccgcggctaaagacggagtcgtggtcaacatgcggtcaatggtaaacagggaccgaggcatcaaggtgtctaggaccggtttatatgctgacgtggacagcgcgtggctatggatcgaggtgttgaataaaacgttggagttagggctgacgccggtttcttggacggattatttgtatttaacggtcggtggaacgttatctaacggcggtataagcggacaaacgtttcggtatggtccacagatcagtaatgttttagagatggatgttattactggtacgtaccatgatgattttttacatgcattctaaatatatgaaacagtaatgacttttacttcgtacatttggcaataaaaaaaatatcgtacgtttttcgtcttttctttaagatctgtgcgctcacttatgtttatgctcttgtttgtttgtaccgtttgtctcttgttctatttatttctaaaacttgttgatagctgttcttcttttcacacatctagctaatcacattattgagtagaaaaacacacgagctatgtgattttatgttttttttctaatttgatactgaattctgttaacaaattatattttttttttttgacaacgttaacaaattatattataagttaagaaccaaatgtttgaaaaattatttctataatttcttacatattatgaacgtttcgctgaatctactgcgatttttacattatggtaataaatgagattagattacgtaactgttcataaaacccggtgaccaaacaaaaaaaaaagacaaatgaaaatcaaccaaaaatggtcccacgcttgatttttcacgaggtttcttgttggcataaggacacaagtcacaagtttataaattttggtcctaaccaggtgtaatggatttaagtaacagtcagttttcttattatagctgtatccattatgtccacacatgcatcattccacatacacctttttgacaacatatatatatatacatacattaatacaactgttgctctcaaatgtagttagttaacgatgattttctagttccattgtgtggttaagagataagataaattaatgagaccgacttcaacactttaggctcaaaacagcgatagtcttatctgaaactcgttgcattagttgaaaaagttttaagagtaactttctaacttggtcagttttgttttacccggaattttataggaaaaggagagattgcaacttgttccaaagacatgaactcagatcttttctatgccgcgttaggaggtttaggtcaattcggaattataacgagagcaagaatcaaactcgaattagctccgaaaaaggtatgttatatatgtaaattatacaataatatttatgttatgtaagctatttaaatctatataaaataataaataaacttatctgcaaattttggaaaaatttcaggctaaatggttaaggtttctatacactgatttctctgagtttacaagagatcaagaacgattgatatcagaaacgaacgggccacatttcctggaaggttctgttatgttagaccatggcccacctgataactggcggtccacttactatccaccgtccgagcacatgaggatcgtctcaatggtcaaacaacatcgtgtcatctactgccttgaagtcgtcaagtattacgacgaaacttcccaacactcagtcaacgaggtccgtacacattatacataattgtacaagtatagtcacaatctttactcataacgaagccactcttccataattataattttattgattgctcaatcatttagacaatgcaggaaatggaggagttaagcgacagtttaaactatgaacgagggtttgtgtacgagaaagatgtgacgtatatggatttcttaaaccgggttcgaaccggagagctaaaactgaaatccaaaggcaagtgggatgttccacatccatggcttaatcttttcgtaccaaaatctcagatttcaagattcgagtatggcgtttttaagggtattatccttagaaataacatcactaccggtccccttcttgtttatcccatgaaccgcaacaagtaagttataatcaatatagccacattatctacagtttcaaaattttcaaatgcgaaatacttaaatctcgaggttttaattcaggtggaatgatcgaatgtcgaccgttatacccgaggaagatgtattttatgctgtaggatttttacgatcggcgagttttgacaattgggatgattatgaaaaagaaaacatggaagtattaaagttttgtgaggatgctaatatgagggtcatacaatatcttccttactatgcatcacaagaaggatgggttggccattttggtccgaggtggaatatttttctcgaaagaaaatatagatatgatcctagaatgatattgtcccctggacaaaatatatttccataa

SEQ ID NO: 8 - DNA, 1560 bp, Brassica napus
  variation (1093)..(1093): C to T substitution in YIIN501
atggtgagctataattttccatcacaaattcatcttctaatgataacgatattagttatcatcacaactcttttaactccgatcacaaccaacacatcatcactaccgtggaatatcctttccaatgacaacttcgccggaaaactcactaccgcatcatcctccgtcgaatcagctgccactgatttcggccacatcaccaaaatctttccctccgccgtcttaaacccttcctccgtccaagacatcaccgatcttataaaactttctttcgattctcaatcctcttttcctctagccgctcgtggccacggacacagccaccgtggtcaagccgcggctaaagacggagtcgtggtcaacatgcggtcaatggtaaacagggaccgaggcatcaaggtgtctaggaccggtttatatgctgacgtggacagcgcgtggctatggatcgaggtgttgaataaaacgttggagttagggctgacgccggtttcttggacggattatttgtatttaacggtcggtggaacgttatctaacggcggtataagcggacaaacgtttcggtatggtccacagatcagtaatgttttagagatggatgttattactggaaaaggagagattgcaacttgttccaaagacatgaactcagatcttttctatgccgcgttaggaggtttaggtcaattcggaattataacgagagcaagaatcaaactcgaattagctccgaaaaaggctaaatggttaaggtttctatacactgatttctctgagtttacaagagatcaagaacgattgatatcagaaacgaacgggccacatttcctggaaggttctgttatgttagaccatggcccacctgataactggcggtccacttactatccaccgtccgagcacatgaggatcgtctcaatggtcaaacaacatcgtgtcatctactgccttgaagtcgtcaagtattacgacgaaacttcccaacactcagtcaacgaggaaatggaggagttaagcgacagtttaaactatgaacgagggtttgtgtacgagaaagatgtgacgtatatggatttcttaaaccgggttcgaaccggagagctaaaactgaaatccaaaggcaagtgggatgttccacatccatggcttaatcttttcgtaccaaaatctcagatttcaagattcgagtatggcgtttttaagggtattatccttagaaataacatcactaccggtccccttcttgtttatcccatgaaccgcaacaagtggaatgatcgaatgtcgaccgttatacccgaggaagatgtattttatgctgtaggatttttacgatcggcgagttttgacaattgggatgattatgaaaaagaaaacatggaagtattaaagttttgtgaggatgctaatatgagggtcatacaatatcttccttactatgcatcacaagaaggatgggttggccattttggtccgaggtggaatatttttctcgaaagaaaatatagatatgatcctagaatgatattgtcccctggacaaaatatatttccataa

SEQ ID NO: 9 - protein, 519 aa, Brassica napus
MVSYNFPSQIHLLMITILVIITTLLTPITTNTSSLPWNILSNDNFAGKLTTASSSVESAATDFGHITKIFPSAVLNPSSVQDITDLIKLSFDSQSSFPLAARGHGHSHRGQAAAKDGVVVNMRSMVNRDRGIKVSRTGLYADVDSAWLWIEVLNKTLELGLTPVSWTDYLYLTVGGTLSNGGISGQTFRYGPQISNVLEMDVITGKGEIATCSKDMNSDLFYAALGGLGQFGIITRARIKLELAPKKAKWLRFLYTDFSEFTRDQERLISETNGPHFLEGSVMLDHGPPDNWRSTYYPPSEHMRIVSMVKQHRVIYCLEVVKYYDETSQHSVNEEMEELSDSLNYERGFVYEKDVTYMDFLNRVRTGELKLKSKGKWDVPHPWLNLFVPKSQISRFEYGVFKGIILRNNITTGPLLVYPMNRNKWNDRMSTVIPEEDVFYAVGFLRSASFDNWDDYEKENMEVLKFCEDANMRVIQYLPYYASQEGWVGHFGPRWNIFLERKYRYDPRMILSPGQNIFP

SEQ ID NO: 10 - DNA, 2991 bp, Brassica napus
  exon (1)..(607): exon1
  exon (1710)..(1834): exon2
  exon (1944)..(2204): exon3
  exon (2311)..(2579): exon4
  variation (2482)..(2482): C to T substitution in YIIN512
  exon (2703)..(2991): exon5
atggcaagtaataattttccatcccaaagtcatcttctagtaataataatatttatcacaactcttttaactccgatcacaaccaacacatcaccacaaccatggaatatcctttccaacgacaacttcgccggaaaactcacctccgcatcctcctccgtcgaagcagcttccatcgatttcggccacgtcaccaaaattctaccctccgccgtcttaaacccttcctccgtccaagacatcatcgatctcataaaactttctttcgactctcaatcctctttccctatagccgctcgtggccacggacacagcttccgtggccaagccgctgctaaagacggagtcgtggtcaacatgcggtcaatggtaaacgaggaccgaggcataaaggtgtctaggaccggtttatatgcggacgtggacactgcgtggctatggattgaggtgttgaataaaacgttggagttggggttaacgccggtttcttggacggactatttgtatttaacggtcggtggaacgttatccaacggcggaataagcggacaaacgtctcggtacggtccacagatcagtaatgttcttgagctggatattattactggtacgtaccatgatttttacatatattccaaatctatgcaatacttacttcgtacgtttgacgacaaaaataaaatacaataaaaacgtacgtttttttagatctgggtggtcacttgtgtttatgctcttgttttgtttaatattgtgtcttttttacgtatttgtcgaacttgttgatagcttttcttgttttcacacatctagctaatcacattatttttgcgtctaagagtagaaagaaacacgggctatgtataaaataagtggttgtaagaaaagatttttctttgttgtagtgatagaagatttagcattattgtttttttgcaacagaaatttcaaattaaaattcgtgaaagatacgtgtaataatatatagtctctacttgaaagtgttgcacaaaaaaaaagtctctacttgaaagttgaaacagaacaatttttttgattttatgttcatattacgattttgtactaacgctatatataacaagttactaactaaatagtttttgaaattttgaaatttataatttcttacatattatggatgtttcgtcgaatctaattttgttctccgcgaaatttacataatggtcacgccgcgcacgttaatgttatgtaattgttcatataaaccggtgacaaacaaatattaaaaatggtcccacgcgtatgttttttcacgaggtttcttgtggcatagagacaaaactcaaaatcttataacgtttggtcataattaaccaggtgtaatgaatttaagtaacagtcaattttcttattatagctaatagttgtaccactatgtccacatatgcatccataattccatatacgaacactgttggtctgaagtttagttcgtttacgaagactttctagttccaaagtttggttaggacttaggagataagatatattaatgggaccgactttaacattttaggcttaaaacaacgatagttttatttgaaactcgttgcatcttttatataaactatgatattctttgtatagtattacggtccaaatatgttgaaatagtttaagactgaactttctaattaggtcagctatttttccccggaactttataggaaaaggggagattgcaacttgttccaacgacatgaactcagatcttttctacgcggcgttaggaggtttgggtcaattcggaattataacaagagccaggattaaactcgaattagctccaaaaagggtatgttatattaatatgtaaattattcaatatatattttatataagtaagaataatttaccgaaatttttgattaaatatatctcttttgttggaaacttttcaggctaaatggttaaggtttctatacactgatttctctgaattcacaagagatcaagaacgattgatatcagaagcgggcggtttacatttcttggaaggttccgttatgcttgaccatggcccacctgataactggagatctacttactatccaccgtccgatcacttgaggatcgtctcaatgataaaacgacaccgtgtcatctactgtctcgaagtcgtcaagtattacgacgaattttctcaacactcagtcaacgaggtccgtacacatatacacaagtataattaggatctccactcacaattaaaaaactctatcttatattatatgtttattaattttctcaattatttaaactttgcaggaaatggaggagttaagcgagagtttaaactttgtaagagggtttgtgtacgagaaagatgtgacgtatatagatttcttgaaccgggttcgaacgggagagctaaacctgaaatccaaaggccaatgggatgttccacatccatggcttaatctctttgtaccaaaatctcagatttcaagatttgattatggtgtctttaagggtattatccttagaaataacatcactaccggtccacttcttgtttatcccatgaagcgcattatgtaagttaaaatatagagcgatcatccattcaattttttatattaaagttcttactaaatgtttatggcctccaaaatttcagctttgtgtttaaataactgacttttaaggttttgcttcaggtggaatgatcaaatgtccaccgctatacccgaagaagatgtattttatgcggtagggttcttacgatctgcggagtttgataattgggaggcttatgataaagaaaatatggaggtattgatgttttgtgaggatgctaaaatggatgtcatacagtatcttccttatcatgcatcacaagaaggatgggttagacattttggtccgaggtggaatacttttgtagagagaaaatataaatacgatcctaaaatgatattgtctccaggacaaaatatatttcaataa

SEQ ID NO: 11 - DNA, 1554 bp, Brassica napus
  variation (1168)..(1168): C to T substitution in YIIN512
atggcaagtaataattttccatcccaaagtcatcttctagtaataataatatttatcacaactcttttaactccgatcacaaccaacacatcaccacaaccatggaatatcctttccaacgacaacttcgccggaaaactcacctccgcatcctcctccgtcgaagcagcttccatcgatttcggccacgtcaccaaaattctaccctccgccgtcttaaacccttcctccgtccaagacatcatcgatctcataaaactttctttcgactctcaatcctctttccctatagccgctcgtggccacggacacagcttccgtggccaagccgctgctaaagacggagtcgtggtcaacatgcggtcaatggtaaacgaggaccgaggcataaaggtgtctaggaccggtttatatgcggacgtggacactgcgtggctatggattgaggtgttgaataaaacgttggagttggggttaacgccggtttcttggacggactatttgtatttaacggtcggtggaacgttatccaacggcggaataagcggacaaacgtctcggtacggtccacagatcagtaatgttcttgagctggatattattactggaaaaggggagattgcaacttgttccaacgacatgaactcagatcttttctacgcggcgttaggaggtttgggtcaattcggaattataacaagagccaggattaaactcgaattagctccaaaaagggctaaatggttaaggtttctatacactgatttctctgaattcacaagagatcaagaacgattgatatcagaagcgggcggtttacatttcttggaaggttccgttatgcttgaccatggcccacctgataactggagatctacttactatccaccgtccgatcacttgaggatcgtctcaatgataaaacgacaccgtgtcatctactgtctcgaagtcgtcaagtattacgacgaattttctcaacactcagtcaacgaggaaatggaggagttaagcgagagtttaaactttgtaagagggtttgtgtacgagaaagatgtgacgtatatagatttcttgaaccgggttcgaacgggagagctaaacctgaaatccaaaggccaatgggatgttccacatccatggcttaatctctttgtaccaaaatctcagatttcaagatttgattatggtgtctttaagggtattatccttagaaataacatcactaccggtccacttcttgtttatcccatgaagcgcattatgtggaatgatcaaatgtccaccgctatacccgaagaagatgtattttatgcggtagggttcttacgatctgcggagtttgataattgggaggcttatgataaagaaaatatggaggtattgatgttttgtgaggatgctaaaatggatgtcatacagtatcttccttatcatgcatcacaagaaggatgggttagacattttggtccgaggtggaatacttttgtagagagaaaatataaatacgatcctaaaatgatattgtctccaggacaaaatatatttcaataa

SEQ ID NO: 12 - protein, 517 aa, Brassica napus
MASNNFPSQSHLLVIIIFITTLLTPITTNTSPQPWNILSNDNFAGKLTSASSSVEAASIDFGHVTKILPSAVLNPSSVQDIIDLIKLSFDSQSSFPIAARGHGHSFRGQAAAKDGVVVNMRSMVNEDRGIKVSRTGLYADVDTAWLWIEVLNKTLELGLTPVSWTDYLYLTVGGTLSNGGISGQTSRYGPQISNVLELDIITGKGEIATCSNDMNSDLFYAALGGLGQFGIITRARIKLELAPKRAKWLRFLYTDFSEFTRDQERLISEAGGLHFLEGSVMLDHGPPDNWRSTYYPPSDHLRIVSMIKRHRVIYCLEVVKYYDEFSQHSVNEEMEELSESLNFVRGFVYEKDVTYIDFLNRVRTGELNLKSKGQWDVPHPWLNLFVPKSQISRFDYGVFKGIILRNNITTGPLLVYPMKRIMWNDQMSTAIPEEDVFYAVGFLRSAEFDNWEAYDKENMEVLMFCEDAKMDVIQYLPYHASQEGWVRHFGPRWNTFVERKYKYDPKMILSPGQNIFQ

SEQ ID NO: 13 - DNA, 2776 bp, Brassica napus
  exon (1)..(613): exon1
  exon (1525)..(1652): exon2
  exon (1759)..(2019): exon3
  variation (1893)..(1893): G to A substitution in YIIN521
  exon (2133)..(2401): exon4
  exon (2488)..(2776): exon5
atggtgagctataattttccatcacaaattcatcttctaatgataacgatattagtaatcatcacaactcttttcactccgatcacaaccaacacatcatcactaccatggaatatcctttccaatgacaacttcgcccgaaaactcactactgcatcatcctccgtcgaatcagctgccactgatttcggccacatcaccaaaatctttccctccgccgtcttaaacccttcctccgtccaagacatcaccgatcttataaaactttctttcgattctcaatcttcttttcctctagccgctcgtggccacggacacagccaccgtggtcaagccgcggctaatgacggagtcgtggtcaacatgcggtcaatggtaaacagggaccgaggcatcaaggtgtctaggacaggtttatatgctgacgtggacagcgcgtggctatggatcgaggtgttgaataaaacgttggagttagggctgacgccggtttcttggacggattatttgcatttaacggtcggtggaacgttatctaacggcggtataagcggacaaacttttcggtatggtccacagatcagtaatgttttagagatggatgttattactggtacgtaccatgatgattttttacatgcattctaaatatatgaaacagtaatgacttttacttcgtacatttggcaataaaaaaaatatcgtacgtttttcgtcttttctttaagatctgtgcgctcactttatgtttatgctcttgtttgtttgtactgtttgtgtcttgttctatttatttctataacttgttgatagctgttcttcttttcacacatttagctaatcacattattgagtagaaaaacacacgagctatgtgattttatggttttttctaatttgataatgaattactgttaaaaaattatattataagttaataaccaaatgtttgaaaatttatttctataatttcttacatattatgacgtttcgctgaatctactgcgaattttacatgatggtaataaatgagatcagattacgtaactgttcataaaacccggtgaccaaacaaacaaaaaagacaaatgaaaatcaaccaaaaatggtcccacacttgatttttcacgaggtttcttgttggcataaggacacaagtcacaagtttataaattttggtcctaaccaggtgtaatggatttaagtaacagtcagttttcttattatagctgtatccattatgtccacacatgcatccattccatatacacctttttgacaacatatatatatatatatatatatacatacattaatacaactgttgctctcaaatgtagttagttaacgatgactttctagttccattgtgtggttaagagataagataaattaatgagaccgacttcaacactttaggctcaaaacagcgatagtctaatctgaaactcgttgcatttgttgaaaaagttttaagagtaactttctaacttggtcagttttgttttacccggaattttataggaaaaggagagattgcaacttgttccaaagacatgaactcagatcttttctatgccgcgttaggaggtttaggtcaattcggaattataacgagagcaagaatcaaactcgaattagctccgaaaaatgtatgttatatatgtaaattatataataatatttatgttatattagctatttaaatctatataaaataataaataaacatatctgcaaattttggaaaattttcaggctaaatggttaaggtttctatacactgatttctctgagtttacaagagatcaagaacgattgatatcagaaacgaacgggctacatttcctggaaggttctgttatgttagaccatggcccacctgataactggcggtccacttactatccaccgtccgagcacatgaggatcgtctcaatggtcaaacaacatcgtgtcatctactgccttgaagtcgtcaagtattacgacgaaacttcccaacactcagtcaacgaggtccgtacacattatacataattgtacaagtatagtcacgatctttactcataacgaagccactcttccataattataattttattgattgatcaatcatttaaacaatgcaggaaatggaggagttaagcgacagtttaaactatgaacgagggtttgtgtacgagaaagatgtgacgtatatggatttcttaaaccgggttcgaaccggagagctaaaactgaaatccaaaggccagtgggatgttccacatccatggcttaatcttttcgtaccaaaatctcagatttcaagattcgagtatggcgtttttaagggtattatccttagaaataacatcactactggtccccttcttgtttatcccatgaaccgcaacaagtaagttatgatcaatatagccacattatctacagtttcaaaattttcaaaatgcgaaatacttaaatctcgaggttttaattcaggtggaatgatcgaatgtcgaccgttttacctgaggaagacgtattttatgctgtcgggtttttacgatcggcgagttttgacaattgggatgattatgaaaaagaaaacatggaagtattaaagttttgcgaggatgctaatatgagggtcatacaatatcttccttactatgcatcacaagaaggatgggttggacattttggtacgaggtggaatatttttctcgaaagaaaatatagatatgatcctagaatgatattgtcccctggacaaaatatatttccataa

SEQ ID NO: 14 - DNA, 1560 bp, Brassica napus
  variation (876)..(876): G to A substitution in YIIN521
atggtgagctataattttccatcacaaattcatcttctaatgataacgatattagtaatcatcacaactcttttcactccgatcacaaccaacacatcatcactaccatggaatatcctttccaatgacaacttcgcccgaaaactcactactgcatcatcctccgtcgaatcagctgccactgatttcggccacatcaccaaaatctttccctccgccgtcttaaacccttcctccgtccaagacatcaccgatcttataaaactttctttcgattctcaatcttcttttcctctagccgctcgtggccacggacacagccaccgtggtcaagccgcggctaatgacggagtcgtggtcaacatgcggtcaatggtaaacagggaccgaggcatcaaggtgtctaggacaggtttatatgctgacgtggacagcgcgtggctatggatcgaggtgttgaataaaacgttggagttagggctgacgccggtttcttggacggattatttgcatttaacggtcggtggaacgttatctaacggcggtataagcggacaaacttttcggtatggtccacagatcagtaatgttttagagatggatgttattactggaaaaggagagattgcaacttgttccaaagacatgaactcagatcttttctatgccgcgttaggaggtttaggtcaattcggaattataacgagagcaagaatcaaactcgaattagctccgaaaaatgctaaatggttaaggtttctatacactgatttctctgagtttacaagagatcaagaacgattgatatcagaaacgaacgggctacatttcctggaaggttctgttatgttagaccatggcccacctgataactggcggtccacttactatccaccgtccgagcacatgaggatcgtctcaatggtcaaacaacatcgtgtcatctactgccttgaagtcgtcaagtattacgacgaaacttcccaacactcagtcaacgaggaaatggaggagttaagcgacagtttaaactatgaacgagggtttgtgtacgagaaagatgtgacgtatatggatttcttaaaccgggttcgaaccggagagctaaaactgaaatccaaaggccagtgggatgttccacatccatggcttaatcttttcgtaccaaaatctcagatttcaagattcgagtatggcgtttttaagggtattatccttagaaataacatcactactggtccccttcttgtttatcccatgaaccgcaacaagtggaatgatcgaatgtcgaccgttttacctgaggaagacgtattttatgctgtcgggtttttacgatcggcgagttttgacaattgggatgattatgaaaaagaaaacatggaagtattaaagttttgcgaggatgctaatatgagggtcatacaatatcttccttactatgcatcacaagaaggatgggttggacattttggtacgaggtggaatatttttctcgaaagaaaatatagatatgatcctagaatgatattgtcccctggacaaaatatatttccataa

SEQ ID NO: 15 - protein, 519 aa, Brassica napus
MVSYNFPSQIHLLMITILVIITTLFTPITTNTSSLPWNILSNDNFARKLTTASSSVESAATDFGHITKIFPSAVLNPSSVQDITDLIKLSFDSQSSFPLAARGHGHSHRGQAAANDGVVVNMRSMVNRDRGIKVSRTGLYADVDSAWLWIEVLNKTLELGLTPVSWTDYLHLTVGGTLSNGGISGQTFRYGPQISNVLEMDVITGKGEIATCSKDMNSDLFYAALGGLGQFGIITRARIKLELAPKNAKWLRFLYTDFSEFTRDQERLISETNGLHFLEGSVMLDHGPPDNWRSTYYPPSEHMRIVSMVKQHRVIYCLEVVKYYDETSQHSVNEEMEELSDSLNYERGFVYEKDVTYMDFLNRVRTGELKLKSKGQWDVPHPWLNLFVPKSQISRFEYGVFKGIILRNNITTGPLLVYPMNRNKWNDRMSTVLPEEDVFYAVGFLRSASFDNWDDYEKENMEVLKFCEDANMRVIQYLPYYASQEGWVGHFGTRWNIFLERKYRYDPRMILSPGQNIFP

SEQ ID NO: 16 - DNA, 2964 bp, Brassica napus
  exon (1)..(610): exon1
  exon (1699)..(1826): exon2
  exon (1928)..(2188): exon3
  variation (2171)..(2171): C to T substitution in YIIN531
  exon (2283)..(2551): exon4
  exon (2676)..(2964): exon5
atggcaagtaataattttccatcacaaagtcatcttctagtaataataatatttatcacaactcttttaactccgatcacaaccaacaacacatcaccacaaccatggaatatcctttccaacgacaacttcgccggaaaactgacctccgcatcctcctccgtcgaagcagctgccatcgatttcggccacgtcaccaaaattctaccctcagccgtcttaaacccttcctccgtccaagacatcatcgatctcataaaactttctttcgactctcaatcctctttccctatagccgctcgtggccacggacacagcttccgtggccaagccgctgctaaagacggagtcgtggtcaacatgcggtcaatggtaaacgaggaccgaggcataaaggtgtctaggaccggtttatatgcggatgtggacactgcatggctatggattgaggtgttgaataaaacgttggagttggggttaacgcctgtttcttggacggactatttgtatttaacgatcggtggaacgttatccaacggcggaataagcggacaaacgtctcggtacggtccacagatcagtaatgttctagagctggatgttattactggtacgtaccatgcttttacatatattccaaatctatgcaatatttacttcgtacgtttgacgacaaaaataaaatacaataaaatcgtacgttttttagatctgcgtggtcacttgtgtttatgctcttgttttgtttatattgtttgtgtcttttgtacatatttctcgaactagcttttcttgttttcacacatctagctaatcacattatttttgtgtctaagagtagaaagaaacacgggctatgtataaaataggtggttgttagaaaagatttttctttgttgtacgtagtgatagaagatttagcattattgctgtttttgcaacaaaaatttcaaattaaaattcgtgaaagatacgtgtaattataatatattgtctctacttgaaagtgttgcacaaaaaaagtgtctacttgaaagttgaaacagaacaatttttttttgattttatgtttttattacgattttgtactaacgctatatataacaagttcaattactaactaaatagtttttcaaattttgaattttataatttcttacatattatggatgtttcgtcgaatctaagtttgatctccgcgaaatttacataatggtcacgccgcgcacgttaatgttatgtaattgttcatataaaccggtgacaaacaaatattcaaccaaaaatggtcccacgcgtatgttttttcacgaggtttcttgtggcatagagacaaaactcaaaagcttataacgtttggtcataaccaggtgtaaattttcttattacagctaatagttgtaccactatgtccacatatgcatccatatataattccatatacaaacactgttggtctcaagtttagttcgtttacgaagactttctagttccaaagtttggttaggagataagatatattaatgggaccgactttaacattttaggcttaaaacaacgatagttttatttgaaactcgttgcatcttttctataaactatgatattctttgtatagtcttacggtccaaatatgttgaaatagtttaagactgaactttctaattgggtcagttatttttccccggaactttataggaaaaggggagattgcaacttgttccaacgacacgaactcagatcttttctacgcggcgttaggaggtttgggtcaattcggaattataacaagagccaggattaaactcgaattagctccaaaaagggtatgttaaatatgtaaattattcaatatatattttatacaagtaagaatatttaccgaaatttttgattaaatatatctcttttgttggaaacttttcaggctaaatggttaaggtttctatacactgatttctctgaattcacaagagatcaagaacgattgatatcagaaacggacggtttacatttcttggaaggttccgttatgcttgaccatggcccacctgataactggagatctacttactatccaccgtccgatcacttgaggatcgtctcaatgatcaaacgataccgtgtcatctactgtctcgaagtcgccaagtattacgacgaaacttctcaacactcagtcaacgaggtccgtacacatatacacgatctctactcataattaaacaactctatcttatattatatgtttcttaattttctcaattatttaaactatgcaggaaattgagaagttaagcgagagtttgaactatgtaagagggtttgtgtacgagaaagatgtgacgtatatagatttcttgaaccgggttcgaacgggagagctaaacctgaaatccaaaggccaatgggatgttccacatccatggcttaatctctttgtaccaaaatctcagatttcaagatttgattatggtgtctttaagggtattatccttagaaataacatcactaccggtccacttcttgtttatcccatgaagcgcattatgtaagttaaaactatagagcgatcatccattcaattttttttattaaagttcttactaaatgtttatggcctccaaaatttcagctttgtgtttaaataactgacttttaaggttttgcttcaggtggaatgatcaaatgtctaccgctatacccgaagaagatgtattttatgcggtagggttcttacgatctgccgagtttgacaattgggaggcttatgataaagaaaatatggaggtattaaagttttgtgaggatgctaaaatggatgtcatacagtatcttccttatcatgcatcacaagaaggatgggttagacattttggtccgatgtggaatacttttgtagagagaaaatatagatacgatcctaaaatgatattgtctccaggacaaaatatatttcgataa

SEQ ID NO: 17 - DNA, 1557 bp, Brassica napus
  variation (982)..(982): C to T substitution in YIIN531
atggcaagtaataattttccatcacaaagtcatcttctagtaataataatatttatcacaactcttttaactccgatcacaaccaacaacacatcaccacaaccatggaatatcctttccaacgacaacttcgccggaaaactgacctccgcatcctcctccgtcgaagcagctgccatcgatttcggccacgtcaccaaaattctaccctcagccgtcttaaacccttcctccgtccaagacatcatcgatctcataaaactttctttcgactctcaatcctctttccctatagccgctcgtggccacggacacagcttccgtggccaagccgctgctaaagacggagtcgtggtcaacatgcggtcaatggtaaacgaggaccgaggcataaaggtgtctaggaccggtttatatgcggatgtggacactgcatggctatggattgaggtgttgaataaaacgttggagttggggttaacgcctgtttcttggacggactatttgtatttaacgatcggtggaacgttatccaacggcggaataagcggacaaacgtctcggtacggtccacagatcagtaatgttctagagctggatgttattactggaaaaggggagattgcaacttgttccaacgacacgaactcagatcttttctacgcggcgttaggaggtttgggtcaattcggaattataacaagagccaggattaaactcgaattagctccaaaaagggctaaatggttaaggtttctatacactgatttctctgaattcacaagagatcaagaacgattgatatcagaaacggacggtttacatttcttggaaggttccgttatgcttgaccatggcccacctgataactggagatctacttactatccaccgtccgatcacttgaggatcgtctcaatgatcaaacgataccgtgtcatctactgtctcgaagtcgccaagtattacgacgaaacttctcaacactcagtcaacgaggaaattgagaagttaagcgagagtttgaactatgtaagagggtttgtgtacgagaaagatgtgacgtatatagatttcttgaaccgggttcgaacgggagagctaaacctgaaatccaaaggccaatgggatgttccacatccatggcttaatctctttgtaccaaaatctcagatttcaagatttgattatggtgtctttaagggtattatccttagaaataacatcactaccggtccacttcttgtttatcccatgaagcgcattatgtggaatgatcaaatgtctaccgctatacccgaagaagatgtattttatgcggtagggttcttacgatctgccgagtttgacaattgggaggcttatgataaagaaaatatggaggtattaaagttttgtgaggatgctaaaatggatgtcatacagtatcttccttatcatgcatcacaagaaggatgggttagacattttggtccgatgtggaatacttttgtagagagaaaatatagatacgatcctaaaatgatattgtctccaggacaaaatatatttcgataa

SEQ ID NO: 18 - protein, 518 aa, Brassica napus
MASNNFPSQSHLLVIIIFITTLLTPITTNNTSPQPWNILSNDNFAGKLTSASSSVEAAAIDFGHVTKILPSAVLNPSSVQDIIDLIKLSFDSQSSFPIAARGHGHSFRGQAAAKDGVVVNMRSMVNEDRGIKVSRTGLYADVDTAWLWIEVLNKTLELGLTPVSWTDYLYLTIGGTLSNGGISGQTSRYGPQISNVLELDVITGKGEIATCSNDTNSDLFYAALGGLGQFGIITRARIKLELAPKRAKWLRFLYTDFSEFTRDQERLISETDGLHFLEGSVMLDHGPPDNWRSTYYPPSDHLRIVSMIKRYRVIYCLEVAKYYDETSQHSVNEEIEKLSESLNYVRGFVYEKDVTYIDFLNRVRTGELNLKSKGQWDVPHPWLNLFVPKSQISRFDYGVFKGIILRNNITTGPLLVYPMKRIMWNDQMSTAIPEEDVFYAVGFLRSAEFDNWEAYDKENMEVLKFCEDAKMDVIQYLPYHASQEGWVRHFGPMWNTFVERKYRYDPKMILSPGQNIFR

SEQ ID NO: 19 - DNA, 2863 bp, Brassica napus
  exon (1)..(580): exon1
  variation (399)..(399): G to A substitution in YIIN805
  variation (465)..(465): G to A substitution in YIIN801
  exon (665)..(792): exon2
  exon (952)..(1218): exon3
  exon (1799)..(2061): exon4
  exon (2506)..(2863): exon5
atgacgtcaagctttcttctcatgacattcgcgatttgtacactgatcatagccgttggtcccagcgagctcctccgcatcggagccatagacgtcgacggccacttcaccttcaagccttccgacttagcctccgtctcgtcggacttcggtatgctaaagtcgccggaggagccattagccgtacttcatccatcctcagctgaagacgtggcacggctcatcagaacagcttacggctcagccacggcctttccggtctcagcccgtggccacggccattccataaacggacaggcttcgacagggaggaacggtgtggtggtggagatgagccaccgcaacatagggacgccggagccactagttgaacccgaggaaatgtatgtggacgtttggggtggagagttatgggttgatgtgttgaaaaaaacgttggagcatggcttagcaccaaagtcatggactgattacttgtatctttccgttggtggtacactctctaatgcaggaattagtggtcaagcttttcaccatggtcctcaaattagtaacgtccttgagctagacgttgtaactggttagtataaaacgttcaagttcatatatttggttcttgaattgattttcaatatttataacaaacaaattgaatcaaaaacagggagaggagaggtgatgagatgttcagaagaagagaacacaaggttattccatggagttcttggtgggttaggtcaatttgggatcattacacgagcacgaatctctctcgaacctgctccccaaagggtaaatgtttttaaattttgattctatgttgtcaagaaataaaaactgccaggcgggtccatgagttacattaacattttcactgttgtggtgacatattttgttttataaatttattaaatttagttaatgaaaccctaaataataatcgtctgacaggtaagatggataagagtgttgtattcgagcttcgaagtgtttacagaggaccaagaatacttaatatctatgcatggtcaatcaaagtttgattacgtggaagggtttgtgatcgtggacgaaggtctcgtcaacaactggagatcatctttcttctctcctcgtaaccccgttaaaatttcttccgttagctctaacggctccgttctgtattgccttgagatcaccaagaactaccgcgagtccgagtccgacaccgtcgatcaggtaaaatttaattaagaagttctcaattttttttctcaattagataaagagatattattctttttataatgatgaatatctggaacgatttaacgctatgcgattattgaggaaatcatgattaaaaaaattggttttcttttagtttatatgattgaaagtggtctccctagtatttttgttgtgtcggctttataatataattaaattgtttattataatttaaattaggaagaagacatgaaaaagaagccagagaggatctacagagatctagcttttccacctgaactattatgtttagtttatcactagcgaattaatcatggttactaataaagtagtggacggggtcatggaccactcaccaaataatgattcccctttactctaattaagttttaataaaaccaactctactggaatcttaacttatccttggttttgatagactttaatagcaaaacagttttttaattttctcctttaactttaattttttttttttttgaaaaatcctttaactttaattttcctagtctggtttaaataaattaaaagtgggttttgtttgctccaggaagtagagattttgatgaagaaattaaattttataccgacatcggtattcacgacggatttgcaatatgtggatttcttggaccgggttcacaaggcggagctgaagctccggtcgaagaatttatgggaggttccacacccatggctcaacctattcgtaccaaaatcgagaatctctgattttgacaaaggcgttttcaagggcattttagggaataaaactagtggccctatccttatctacccaatgaacaaagacaagtaagtctcgatcgatattaccagcgtttacttagaaatttcttttcagaaacaaaagaataaaacgagttttgcatgcatgcaaagttacacttgtggggattaaattagtggtccaagaaaataaatgttttgtcgaaactgtaaagaactactgacacgtggtacatgggattgtccgaaaaactttgttcacatgtgcatcgaatcgaaccagctaagattgataataacgcatcatcgtcggctcatctttctcttttggttcgttgtccaatctggacggtccagattgggtctattgttttttagttcctataactaatggtggtgggtcccaatcggattctactaaaccaaaccgatctcaacgaccctccctcacatcatttattaactataagattatgtcgtgctgacgtgacgtactttttgtaatgccagatgggacgaaagaagctcggccgttacaccggacgaggaagttttctacctcgtggctttactgagatcagctttatcggacggtgaggaaacgcagaagctagagtatttgaaagatcagaaccgtcggatcttggagttctgtgaacaagccaagatcaatgtgaagcagtaccttcctcaccatgcaacgcaagaggagtgggtggctcattatggagacaaatgggatcagtttagaagcatgaaggctgagttcgatccacggcatatactcgctaccggtcagaaaatctttcagaacccatctctggctttgtttcgtccatcgtcgtcttcgtcgtcagcttcatggtaa

SEQ ID NO: 20 - DNA, 1596 bp, Brassica napus
  variation (399)..(399): G to A substitution in YIIN805
  variation (465)..(465): G to A substitution in YIIN801
atgacgtcaagctttcttctcatgacattcgcgatttgtacactgatcatagccgttggtcccagcgagctcctccgcatcggagccatagacgtcgacggccacttcaccttcaagccttccgacttagcctccgtctcgtcggacttcggtatgctaaagtcgccggaggagccattagccgtacttcatccatcctcagctgaagacgtggcacggctcatcagaacagcttacggctcagccacggcctttccggtctcagcccgtggccacggccattccataaacggacaggcttcgacagggaggaacggtgtggtggtggagatgagccaccgcaacatagggacgccggagccactagttgaacccgaggaaatgtatgtggacgtttggggtggagagttatgggttgatgtgttgaaaaaaacgttggagcatggcttagcaccaaagtcatggactgattacttgtatctttccgttggtggtacactctctaatgcaggaattagtggtcaagcttttcaccatggtcctcaaattagtaacgtccttgagctagacgttgtaactgggagaggagaggtgatgagatgttcagaagaagagaacacaaggttattccatggagttcttggtgggttaggtcaatttgggatcattacacgagcacgaatctctctcgaacctgctccccaaagggtaagatggataagagtgttgtattcgagcttcgaagtgtttacagaggaccaagaatacttaatatctatgcatggtcaatcaaagtttgattacgtggaagggtttgtgatcgtggacgaaggtctcgtcaacaactggagatcatctttcttctctcctcgtaaccccgttaaaatttcttccgttagctctaacggctccgttctgtattgccttgagatcaccaagaactaccgcgagtccgagtccgacaccgtcgatcaggaagtagagattttgatgaagaaattaaattttataccgacatcggtattcacgacggatttgcaatatgtggatttcttggaccgggttcacaaggcggagctgaagctccggtcgaagaatttatgggaggttccacacccatggctcaacctattcgtaccaaaatcgagaatctctgattttgacaaaggcgttttcaagggcattttagggaataaaactagtggccctatccttatctacccaatgaacaaagacaaatgggacgaaagaagctcggccgttacaccggacgaggaagttttctacctcgtggctttactgagatcagctttatcggacggtgaggaaacgcagaagctagagtatttgaaagatcagaaccgtcggatcttggagttctgtgaacaagccaagatcaatgtgaagcagtaccttcctcaccatgcaacgcaagaggagtgggtggctcattatggagacaaatgggatcagtttagaagcatgaaggctgagttcgatccacggcatatactcgctaccggtcagaaaatctttcagaacccatctctggctttgtttcgtccatcgtcgtcttcgtcgtcagcttcatggtaa

SEQ ID NO: 21 - protein, 531 aa, Brassica napus
MTSSFLLMTFAICTLIIAVGPSELLRIGAIDVDGHFTFKPSDLASVSSDFGMLKSPEEPLAVLHPSSAEDVARLIRTAYGSATAFPVSARGHGHSINGQASTGRNGVVVEMSHRNIGTPEPLVEPEEMYVDVWGGELWVDVLKKTLEHGLAPKSWTDYLYLSVGGTLSNAGISGQAFHHGPQISNVLELDVVTGRGEVMRCSEEENTRLFHGVLGGLGQFGIITRARISLEPAPQRVRWIRVLYSSFEVFTEDQEYLISMHGQSKFDYVEGFVIVDEGLVNNWRSSFFSPRNPVKISSVSSNGSVLYCLEITKNYRESESDTVDQEVEILMKKLNFIPTSVFTTDLQYVDFLDRVHKAELKLRSKNLWEVPHPWLNLFVPKSRISDFDKGVFKGILGNKTSGPILIYPMNKDKWDERSSAVTPDEEVFYLVALLRSALSDGEETQKLEYLKDQNRRILEFCEQAKINVKQYLPHHATQEEWVAHYGDKWDQFRSMKAEFDPRHILATGQKIFQNPSLALFRPSSSSSSASW

SEQ ID NO: 22 - DNA, 2804 bp, Brassica napus
  exon (1)..(580): exon1
  variation (465)..(465): G to A substitution in YIIN811
  exon (664)..(791): exon2
  exon (950)..(1216): exon3
  exon (1765)..(2027): exon4
  exon (2447)..(2804): exon5
atgacgtcaagctttcttctcgtgacattcgcgatttgtacactgatcatagccgttggtcccagcgagctcctccgcatcggagccatagacgtcgacggccacttcaccttcaagccttccgacttagcctccgtctcgtcggacttcggtatgctaaagtcgccggaggagccattagccgtacttcatccatcctcagctgaagacgtggcacggctcatcagaacagcttacggctcagccacggcctttccggtctcagcccgtggccacggccattccataaacggacaggcttcgacagggaggaacggtgtggtggtggagatgagccaccgcaacatagggacgctggagccactagttgaaccggaggaaatgtatgtggacgtttggggtggagagttatgggttgatgtgttgaaaaaaacgttggagcatggcttagcaccaaagtcatggactgattacctgtatctttccgttggtggtacactctccaatgcaggaattagtggtcaagcttttcaccatggtcctcaaattagtaacgtccttgagctagacgttgtaactggttagtataaaacgttcaagttcatatatttggttcttgaattgattttcatattaataacaaacatattgaatcaaaaacagggagaggagaggtgatgagatgttcagaagaagagaacacaaggcttttccatggagttcttggtgggttaggtcaatttgggatcattacacgagcacgaatctctctcgaacctgctccccaaagggtaaatgtttttaaattttgattctatgttgtcacgatataaaactgccaggcgggtccatgagttacattaaccttttcactattgtggtgacatattttgttttataaatttattaaatttagttaatgaaaccctaaatgataatcttgtgacaggtaagatggataagagtgttgtattcgagcttcgaagtgtttacagatgaccaagaatacttaatatctatgcatggtcattcaaagtttgattacgtggaagggtttgtgatcgtggacgaaggtctcgtcaacaactggagatcctctttcttctctcctcataaccccgttaaaatttcttccgttagctctaacggctccgttctgtattgccttgagatcaccaagaactaccacgagtccgagtccgacaccgtcgatcaggtaaaatttaattaagaagttctcaattttttttcttctcaattagataaagagatattattctttttataatgatgaatatctggaacgatttaacgctatgcgattattgaggaaatcatgattaaaaaatttggttttcttttagtttatatgattgaaagtggtctccatagtatttttgttgtgtcggctttattatataattaaaatagtttattataatttaaattaggaagaaggcatgaaaaagaagcgagagaggatctacagagatctagcttttccacctgaactattatgtttagtttatcactagcgaattaatcatggttactaattaagtagtggacggggtcatggaccactcaccaaataatgattcccctttactctaattaagttttaataaaaccaactctactggaatcttaacttatccttggttttggtaggctttaatagcaaaacagttttttaattttctcctaactttaattttcctattctggtttaaataaattaaaagtgggttttgtttgctccaggaagtagagattttgatgaagaaattaaattttataccgacatcggtattcacgacagatttgcaatatgtggatttcctggaccgggttcacaaggcggagctgaagctccggtcgaagaatttatgggaggttccacacccatggctcaacctattcgtaccaaaatcaagaatctctgattttgacaaaggcgttttcaagggcattttggggaataaaactagtggccctatccttatctacccaatgaacaaagacaagtaagttccgatcgatattaccaccgtttacttagaaatttcttttcagaaacaaaagaataaaacgagttttgcatgcatgcaaagttacacttgtggtccaaaaaaataaatgttttgtcgaaactgtaaattagtggtacatgggattgtccgaaaaactttgttcacatgtgcatcgaatcgaaccagctaagattgataataacgcatcatcgtcggctcatctttctcttttggttcattgtccgatctggacggtccagattgggtctattgttttttagttcctataactaatggtggtgggtcccaatcggattctacgacacggaaccgatctcaacgaccctcccccacatcatttattaactataagattatgtcgtgctgacgtgtcggattttttgtaatgtcagatgggacgaaagaagctcggccgttacaccggacgaggaagttttctacctggtggctctattgagatcagctttatcggatggtgaggaaacgcagaagctagagtatttgaaagatcagaaccgtcggatcttggagttctgtgaacaagccaagatcaatgtgaagcagtaccttcctcaccatgcaacgcaagaggagtgggtggatcattatggagacagatgggatcagtttagaagcatgaagactgagttcgatccacggcatatactcgctaccggtcagaaaatctttcagaacccatctctggctttgtttcctccatcgccgtcttcatcgtcagcttcatggtga

SEQ ID NO: 23 - DNA, 1596 bp, Brassica napus
  variation (465)..(465): G to A substitution in YIIN811
atgacgtcaagctttcttctcgtgacattcgcgatttgtacactgatcatagccgttggtcccagcgagctcctccgcatcggagccatagacgtcgacggccacttcaccttcaagccttccgacttagcctccgtctcgtcggacttcggtatgctaaagtcgccggaggagccattagccgtacttcatccatcctcagctgaagacgtggcacggctcatcagaacagcttacggctcagccacggcctttccggtctcagcccgtggccacggccattccataaacggacaggcttcgacagggaggaacggtgtggtggtggagatgagccaccgcaacatagggacgctggagccactagttgaaccggaggaaatgtatgtggacgtttggggtggagagttatgggttgatgtgttgaaaaaaacgttggagcatggcttagcaccaaagtcatggactgattacctgtatctttccgttggtggtacactctccaatgcaggaattagtggtcaagcttttcaccatggtcctcaaattagtaacgtccttgagctagacgttgtaactgggagaggagaggtgatgagatgttcagaagaagagaacacaaggcttttccatggagttcttggtgggttaggtcaatttgggatcattacacgagcacgaatctctctcgaacctgctccccaaagggtaagatggataagagtgttgtattcgagcttcgaagtgtttacagatgaccaagaatacttaatatctatgcatggtcattcaaagtttgattacgtggaagggtttgtgatcgtggacgaaggtctcgtcaacaactggagatcctctttcttctctcctcataaccccgttaaaatttcttccgttagctctaacggctccgttctgtattgccttgagatcaccaagaactaccacgagtccgagtccgacaccgtcgatcaggaagtagagattttgatgaagaaattaaattttataccgacatcggtattcacgacagatttgcaatatgtggatttcctggaccgggttcacaaggcggagctgaagctccggtcgaagaatttatgggaggttccacacccatggctcaacctattcgtaccaaaatcaagaatctctgattttgacaaaggcgttttcaagggcattttggggaataaaactagtggccctatccttatctacccaatgaacaaagacaaatgggacgaaagaagctcggccgttacaccggacgaggaagttttctacctggtggctctattgagatcagctttatcggatggtgaggaaacgcagaagctagagtatttgaaagatcagaaccgtcggatcttggagttctgtgaacaagccaagatcaatgtgaagcagtaccttcctcaccatgcaacgcaagaggagtgggtggatcattatggagacagatgggatcagtttagaagcatgaagactgagttcgatccacggcatatactcgctaccggtcagaaaatctttcagaacccatctctggctttgtttcctccatcgccgtcttcatcgtcagcttcatggtga

SEQ ID NO: 24 - protein, 531 aa, Brassica napus
MTSSFLLVTFAICTLIIAVGPSELLRIGAIDVDGHFTFKPSDLASVSSDFGMLKSPEEPLAVLHPSSAEDVARLIRTAYGSATAFPVSARGHGHSINGQASTGRNGVVVEMSHRNIGTLEPLVEPEEMYVDVWGGELWVDVLKKTLEHGLAPKSWTDYLYLSVGGTLSNAGISGQAFHHGPQISNVLELDVVTGRGEVMRCSEEENTRLFHGVLGGLGQFGIITRARISLEPAPQRVRWIRVLYSSFEVFTDDQEYLISMHGHSKFDYVEGFVIVDEGLVNNWRSSFFSPHNPVKISSVSSNGSVLYCLEITKNYHESESDTVDQEVEILMKKLNFIPTSVFTTDLQYVDFLDRVHKAELKLRSKNLWEVPHPWLNLFVPKSRISDFDKGVFKGILGNKTSGPILIYPMNKDKWDERSSAVTPDEEVFYLVALLRSALSDGEETQKLEYLKDQNRRILEFCEQAKINVKQYLPHHATQEEWVDHYGDRWDQFRSMKTEFDPRHILATGQKIFQNPSLALFPPSPSSSSASW

SEQ ID NO: 25 - protein, 364 aa, Brassica napus
MVSYNFPSQIHLLMITILVIITTLLTPITTNTSSLPWNILSNDNFAGKLTTASSSVESAATDFGHITKIFPSAVLNPSSVQDITDLIKLSFDSQSSFPLAARGHGHSHRGQAAAKDGVVVNMRSMVNRDRGIKVSRTGLYADVDSAWLWIEVLNKTLELGLTPVSWTDYLYLTVGGTLSNGGISGQTFRYGPQISNVLEMDVITGKGEIATCSKDMNSDLFYAALGGLGQFGIITRARIKLELAPKKAKWLRFLYTDFSEFTRDQERLISETNGPHFLEGSVMLDHGPPDNWRSTYYPPSEHMRIVSMVKQHRVIYCLEVVKYYDETSQHSVNEEMEELSDSLNYERGFVYEKDVTYMDFLNRV

SEQ ID NO: 26 - protein, 389 aa, Brassica napus
MASNNFPSQSHLLVIIIFITTLLTPITTNTSPQPWNILSNDNFAGKLTSASSSVEAASIDFGHVTKILPSAVLNPSSVQDIIDLIKLSFDSQSSFPIAARGHGHSFRGQAAAKDGVVVNMRSMVNEDRGIKVSRTGLYADVDTAWLWIEVLNKTLELGLTPVSWTDYLYLTVGGTLSNGGISGQTSRYGPQISNVLELDIITGKGEIATCSNDMNSDLFYAALGGLGQFGIITRARIKLELAPKRAKWLRFLYTDFSEFTRDQERLISEAGGLHFLEGSVMLDHGPPDNWRSTYYPPSDHLRIVSMIKRHRVIYCLEVVKYYDEFSQHSVNEEMEELSESLNFVRGFVYEKDVTYIDFLNRVRTGELNLKSKGQWDVPHPWLNLFVPKS

SEQ ID NO: 27 - protein, 291 aa, Brassica napus
MVSYNFPSQIHLLMITILVIITTLFTPITTNTSSLPWNILSNDNFARKLTTASSSVESAATDFGHITKIFPSAVLNPSSVQDITDLIKLSFDSQSSFPLAARGHGHSHRGQAAANDGVVVNMRSMVNRDRGIKVSRTGLYADVDSAWLWIEVLNKTLELGLTPVSWTDYLHLTVGGTLSNGGISGQTFRYGPQISNVLEMDVITGKGEIATCSKDMNSDLFYAALGGLGQFGIITRARIKLELAPKNAKWLRFLYTDFSEFTRDQERLISETNGLHFLEGSVMLDHGPPDN

SEQ ID NO: 28 - protein, 327 aa, Brassica napus
MASNNFPSQSHLLVIIIFITTLLTPITTNNTSPQPWNILSNDNFAGKLTSASSSVEAAAIDFGHVTKILPSAVLNPSSVQDIIDLIKLSFDSQSSFPIAARGHGHSFRGQAAAKDGVVVNMRSMVNEDRGIKVSRTGLYADVDTAWLWIEVLNKTLELGLTPVSWTDYLYLTIGGTLSNGGISGQTSRYGPQISNVLELDVITGKGEIATCSNDTNSDLFYAALGGLGQFGIITRARIKLELAPKRAKWLRFLYTDFSEFTRDQERLISETDGLHFLEGSVMLDHGPPDNWRSTYYPPSDHLRIVSMIKRYRVIYCLEVAKYYDETS

SEQ ID NO: 29 - protein, 154 aa, Brassica napus
MTSSFLLMTFAICTLIIAVGPSELLRIGAIDVDGHFTFKPSDLASVSSDFGMLKSPEEPLAVLHPSSAEDVARLIRTAYGSATAFPVSARGHGHSINGQASTGRNGVVVEMSHRNIGTPEPLVEPEEMYVDVWGGELWVDVLKKTLEHGLAPKS

SEQ ID NO: 30 - protein, 132 aa, Brassica napus
MTSSFLLMTFAICTLIIAVGPSELLRIGAIDVDGHFTFKPSDLASVSSDFGMLKSPEEPLAVLHPSSAEDVARLIRTAYGSATAFPVSARGHGHSINGQASTGRNGVVVEMSHRNIGTPEPLVEPEEMYVDV

SEQ ID NO: 31 - protein, 154 aa, Brassica napus
MTSSFLLVTFAICTLIIAVGPSELLRIGAIDVDGHFTFKPSDLASVSSDFGMLKSPEEPLAVLHPSSAEDVARLIRTAYGSATAFPVSARGHGHSINGQASTGRNGVVVEMSHRNIGTLEPLVEPEEMYVDVWGGELWVDVLKKTLEHGLAPKS

SEQ ID NO: 32 - DNA, 47 bp, Artificial Sequence
  KASP Primer BnCKX3-A1 WT allele
gaaggtgaccaagttcatgctgtatatggatttcttaaaccgggttc

SEQ ID NO: 33 - DNA, 48 bp, Artificial Sequence
  KASP Primer BnCKX3-A1 YIIN501 allele
gaaggtcggagtcaacggattcgtatatggatttcttaaaccgggttt

SEQ ID NO: 34 - DNA, 48 bp, Artificial Sequence
  KASP Primer BnCKX3-A2 WT allele
gaaggtgaccaagttcatgctggcttaatctctttgtaccaaaatctc

SEQ ID NO: 35 - DNA, 50 bp, Artificial Sequence
  KASP Primer BnCKX3-A2 YIIN512  allele
gaaggtcggagtcaacggattatggcttaatctctttgtaccaaaatctt

SEQ ID NO: 36 - DNA, 42 bp, Artificial Sequence
  KASP Primer BnCKX3-C1 WT allele
gaaggtgaccaagttcatgctggtggatagtaagtggaccgc

SEQ ID NO: 37 - DNA, 43 bp, Artificial Sequence
  KASP Primer BnCKX3-C1 YIIN521 allele
gaaggtcggagtcaacggattcggtggatagtaagtggaccgt

SEQ ID NO: 38 - DNA, 43 bp, Artificial Sequence
  KASP Primer BnCKX3-C2 WT allele
gaaggtgaccaagttcatgctggacctcgttgactgagtgttg

SEQ ID NO: 39 - DNA, 44 bp, Artificial Sequence
  KASP Primer BnCKX3-C2 YIIN531 allele
gaaggtcggagtcaacggattcggacctcgttgactgagtgtta

SEQ ID NO: 40 - DNA, 47 bp, Artificial Sequence
  KASP Primer BnCKX5-A1 WT allele
gaaggtgaccaagttcatgctcaacggaaagatacaagtaatcagtc

SEQ ID NO: 41 - DNA, 48 bp, Artificial Sequence
  KASP Primer BnCKX5-YIIN801 WT allele
gaaggtcggagtcaacggattccaacggaaagatacaagtaatcagtt

SEQ ID NO: 42 - DNA, 44 bp, Artificial Sequence
  KASP Primer BnCKX5-A1 WT allele
gaaggtgaccaagttcatgctcatcaacccataactctccaccc

SEQ ID NO: 43 - DNA, 45 bp, Artificial Sequence
  KASP Primer BnCKX5-A1 YIIN805 allele
gaaggtcggagtcaacggattacatcaacccataactctccacct

SEQ ID NO: 44 - DNA, 47 bp, Artificial Sequence
  KASP Primer BnCKX5C1 WT allele
gaaggtgaccaagttcatgctcaacggaaagatacaggtaatcagtc

SEQ ID NO: 45 - DNA, 47 bp, Artificial Sequence
  KASP Primer BnCKX5-C1 YIIN811 allele
gaaggtcggagtcaacggattcaacggaaagatacaggtaatcagtt

(19) United States
(12) Patent Application Publication
(10) Pub. No.: US 2019/0153456 A1
(43) Pub. Date: May 23, 2019

(54) BRASSICA PLANTS WITH ALTERED PROPERTIES IN SEED PRODUCTION

(71) Applicant: BAYER CROPSCIENCE NV, Diegem (BE)

(72) Inventors: Freya LAMMERTYN, Wachtebeke (BE); Marc BOTS, Utrecht (NL); Benjamin LAGA, Wingene (BE); Ralf-Christian SCHMIDT, Deurle (BE); Julia SCHMIDT, Deurle (BE); Celine MOUCHEL, De Pinte (BE)

(21) Appl. No.: 15/768,273

(22) PCT Filed: Oct. 13, 2016
(86) PCT No.: PCT/EP2016/074560

§ 371 Date: Apr. 13, 2018

(30) Foreign Application Priority Data
Oct. 16, 2015 (EP) 15190256.6

(51) Int. Cl. C12N 15/82; C12N 9/06

(52) U.S. Cl. CPC C12N 15/827; C12Y 105/99012; C12N 9/0026; C12N 15/8218; C12N 15/8295; C12N 9/0022

(57) ABSTRACT

The present invention relates to plants having increased number of flowers, pod and increased thousand seed weight (TSW). More specifically, the invention relates to Brassica plants in which expression of Cytokinin oxidase 5 or Cytokinin oxidase 5 and 3 is functionally reduced. Provided are Brassica plants comprising mutant CKX alleles, and Brassica plants in which expression of CKX is reduced. Also provided are methods and means to produce Brassica plants with increased number of flowers, pod or TSW.

### FIELD OF THE INVENTION

This invention relates to Brassica plants and parts, particularly Brassica napus plants, with altered flower number, pod number and seed production characteristics. The invention also relates to nucleic acids encoding cytokinin oxidases (CKX) from Brassica napus, and induced variant alleles thereof that affect flower, pod and seed production in Brassica napus plants.

### BACKGROUND OF THE INVENTION

Increasing productivity in agriculture is a continuous goal in order to meet the growing demand for food, feed and other plant derived product in view of growing human population and continuous decrease in land space with optimal characteristics which can be allocated to agriculture.

Cytokinin is a plant hormone that affects many aspects of plant growth and development. It stimulates the formation and activity of shoot meristems, is able to establish sink tissues, delay leaf senescence, inhibit root growth and branching, and plays a role in seed germination and stress responses (Mok and Mok, 2001, Ann. Rev. Plant Physiol. Mol. Biol. 52, 89-118). The chemistry and physiology of cytokinin have been studied extensively, as well as the regulation of cytokinin biosynthesis, metabolism, and signal transduction.

Cytokinin oxidases (CKX), also referred to as cytokinin dehydrogenases, regulate homeostasis of the plant hormone cytokinin. They catalyze the irreversible degradation of the cytokinins isopentenyladenine, zeatin, and their ribosides in a single enzymatic step by oxidative side chain cleavage. The genome of Arabidopsis thaliana encodes seven CKX genes, while the genome of rice comprises at least ten members of the CKX family. Individual CKX proteins differ in their catalytic properties, their subcellular localization and their expression patterns with regard to timing, developmental stage and tissue. CKX enzymes are responsible for most cytokinin catabolism and inactivate the hormone. Because changes in CKX protein level or functionality and subsequent changes in CKX activity alter the cytokinin concentration in tissues, CKX enzymes are important in controlling local cytokinin levels and contribute to the regulation of cytokinin-dependent processes. (Schmulling et al., 2003, J. Plant Res. 116, 241-252). Modulation of CKX gene expression and CKX protein activity has been used in biotechnological applications to alter plant morphology, biochemistry, physiology and development.

WO2001/96580 describes methods for stimulating root growth and/or enhancing the formation of lateral or adventitious roots and/or altering root geotropism comprising expression of a plant cytokinin oxidase or comprising expression of another protein that reduces the level of active cytokinins in plants or plant parts. Also described are novel plant cytokinin oxidase proteins, nucleic acid sequences encoding cytokinin oxidase proteins as well as to vectors, host cells, transgenic cells and plants comprising such sequences. The document also describes the use of these sequences for improving root-related characteristics including increasing yield and/or enhancing early vigor and/or modifying root/shoot ratio and/or improving resistance to lodging and/or increasing drought tolerance and/or promoting in vitro propagation of explants and/or modifying cell fate and/or plant development and/or plant morphology and/or plant biochemistry and/or plant physiology. Further described are the use of these sequences in the above-mentioned methods. Methods for identifying and obtaining proteins and compounds interacting with cytokinin oxidase proteins are disclosed as well as the use of such compounds as a plant growth regulator or herbicide.

WO2003/050287 also describes methods for stimulating root growth and/or enhancing the formation of lateral or adventitious roots and/or altering root geotropism comprising expression of a plant cytokinin oxidase or comprising expression of another protein that reduces the level of active cytokinins in plants or plant parts. Also provided are methods for increasing seed size and/or weight, embryo size and/or weight, and cotyledon size and/or weight. The methods comprise expression of a plant cytokinin oxidase or expression of another protein that reduces the level of active cytokinins in plants or plant parts. The document further describes novel plant cytokinin oxidase proteins, nucleic acid sequences encoding cytokinin oxidase proteins as well as to vectors, host cells, transgenic cells and plants comprising said sequences. Also disclosed are the use of such sequences for improving root-related characteristics including increasing yield and/or enhancing early vigor and/or modifying root/shoot ratio and/or improving resistance to lodging and/or increasing drought tolerance and/or promoting in vitro propagation of explants and/or modifying cell fate and/or plant development and/or plant morphology and/or plant biochemistry and/or plant physiology. Finally the described technology also relates to the use of such sequences in the above-mentioned methods as well as methods for identifying and obtaining proteins and compounds interacting with cytokinin oxidase proteins and use of such compounds as a plant growth regulator or herbicide.

WO2005/123926 describes methods and compositions for increasing seed yield of a plant. The methods comprise expression of a cytokinin oxidase in the aleurone and/or embryo of a seed. Further described are vectors comprising a nucleic acid encoding a cytokinin oxidase that is operably linked to a promoter capable of driving expression in the aleurone and/or embryo of a seed, and to host cells, transgenic cells and plants comprising such sequences. The use of these sequences for increasing yield is also provided.

US2006123507 describes a CKX gene that regulates the increase and decrease of the particle-bearing number (including glumous flowers, fruits, and seeds) of cereal plants which was successfully isolated and identified by a linkage analysis. In addition, breeding methods that utilize this gene to increase the particle-bearing number (including glumous flowers, fruits, and seeds) of plants were also discovered.

US2013/014291 describes cytokining oxidase like sequences (from Zea mays) and methods of use. The sequences can be used in a variety of methods including modulating root development, modulating floral development, modulating leaf and/or shoot development, modulating seed size and/or weight, modulating tolerance under abiotic stress, and modulating resistance to pathogens.

Cervinkova et al. (2013 J. Exp. Bot. 64, 2805-2815) described enhanced drought and heat stress tolerance of tobacco plants with ectopically enhanced cytokinin oxidase/dehydrogenase gene expression.

Köllmer et al. (2014, Plant J. 78, 359-371) reported that overexpression of the cytosolic cytokinin oxidase/dehydrogenase (CKX7) from Arabidopsis causes specific changes in root growth and xylem differentiation.

WO2011/004003 is directed to isolated plant cells and plants comprising a disruption in at least a CKX3 gene and in one further gene encoding for a cytokininoxidase/dehydrogenase and being different from CKX3 well as to methods of producing such plants and to methods of increasing seed yield in a plant and/or plant height.

Batrina et al. (2011, The Plant Cell 23, 69-80) report that the size and activity of the shoot apical meristem is regulated by transcription factors and low molecular mass signals, including the plant hormone cytokinin. The cytokinin status of the meristem depends on different factors, including metabolic degradation of the hormone, which is catalyzed by cytokinin oxidase/dehydrogenase (CKX) enzymes. In this document they report that CKX3 and CKX5 regulate the activity of the reproductive meristems of Arabidopsis thaliana. CKX3 is expressed in the central WUSCHEL (WUS) domain, while CKX5 shows a broader meristematic expression. ckx3 ckx5 double mutants in Arabidopsis thaliana form larger inflorescence and floral meristems. An increased size of the WUS domain and enhanced primordia formation indicate a dual function for cytokinin in defining the stem cell niche and delaying cellular differentiation. Consistent with this, mutation of a negative regulator gene of cytokinin signaling, ARABIDOPSIS HISTIDINE PHOSPHOTRANSFER PROTEIN 6, which is expressed at the meristem flanks, caused a further delay of differentiation. Terminal cellular differentiation was also retarded in ckx3 ckx5 flowers, which formed more cells and became larger, corroborating the role of cytokinin in regulating flower organ size. Furthermore, higher activity of the ckx3 ckx5 placenta tissue established supernumerary ovules leading to an increased seed set per silique. Together, the results underpin the important role of cytokinin in reproductive development. The increased cytokinin content caused an ˜55% increase in seed yield in Arabidopsis thaliana, highlighting the relevance of sink strength as a yield factor.

Song et et al. (2015 J. Exp. Bot. 66 pp 5067-5082) report that expression patterns of Brassica napus genes implicate IPT, CKX, sucrose transporter, cell wall invertase and amino acid permease gene family members in leaf, flower, silique, and seed development. The publication reports the identification of a gene family for cytokinin degradation (BnCKX1 to BnCKX7) in B. napus, as well as the homeologues from B. oleracea and B. rapa. No accession numbers for the sequences are published (although the supplementary data annex 1 purports to list such numbers). BnCKX2 and 4 were identified as targets for TILLING, EcoTILLING and MAS in an effort to improve seed yield without affecting forage yield and quality in forage brassica (Brassica napus cv. Greenland) which is bred for vegetative growth and biomass production.

There thus remains a need for non-functional variant alleles of CKX3 and CKX5 genes from B. napus which can be used to alter flower production, pod production and seed production as well as seed characteristics in Brassica napus.

### SUMMARY OF THE INVENTION

The inventors have found that by controlling the number and/or types of CKX5 or CKX5 and CKX3 genes alleles that are “functionally expressed” in said plants, i.e. that result in functional (biologically active) CKX5 or CKX5 and CKX3 protein in Brassica plants, the number of flowers, pods and seeds per plant can be modulated.

By combining certain induced variant alleles of the CKX5 or CKX5 and CKX3 genes, resulting in a reduction of the level of functional CKX5 or CKX5 and CKX3 protein, the number of flowers per plant can be increased, particularly the number of flowers on the main branch can be increased under greenhouse or field trial conditions. Furthermore, the number of pods on the main branch can be increased in field trial conditions, as well as the number of seeds per pod on the main branch. Also an increase in Thousand Seed Weight (TSW) can be achieved, particularly a higher TSW without a significant negative effect on seed yield, contrary to other approaches yielding a higher TSW but compensating this with a lower seed number yield.

In one embodiment, a Brassica plant comprising at least one CKX5 gene, comprising at least one mutant CKX5 allele in its genome is provided, particularly wherein said mutant CKX5 allele is a mutant allele of a CKX5 gene comprising a nucleic acid sequence selected from the group consisting of: a nucleotide sequence which comprises at least 90% sequence identity to SEQ ID NO: 19 or SEQ ID NO: 23; a nucleotide sequence comprising a coding sequence which comprises at least 90% sequence identity to SEQ ID NO: 20 or SEQ ID NO: 23; and a nucleotide sequence encoding an amino acid sequence which comprises at least 90% sequence identity to SEQ ID NO: 21, or SEQ ID NO: 24.

The Brassica plant may comprise two CKX5 genes and be selected from the group consisting of Brassica napus, Brassica juncea and Brassica carinata. The plant may also comprise at least two mutant CKX5 alleles, or at least three mutant CKX5 alleles, or at least four mutant CKX5 alleles.

In a particular embodiment the mutant CKX5 allele may be selected from: a mutant CKX5 allele comprising a G to A substitution at a position corresponding to position 465 of SEQ ID NO: 19 or position 465 pf SEQ ID No. 20; a mutant CKX5 allele comprising a G to A substitution at a position corresponding to position 399 of SEQ ID NO: 19 or position 399 of SEQ ID No. 20; and a mutant CKX5 allele comprising a G to A substitution at a position corresponding to position 465 of SEQ ID NO: 22 or position 399 of SEQ ID No. 23.

In _{y}et another embodiment, the plant may further comprise at least two CKX3 genes, further comprising at least two mutant CKX3 alleles in its genome, particularly wherein said mutant CKX3 allele is a mutant allele of a CKX3 gene comprising a nucleic acid sequence selected from the group consisting of: a nucleotide sequence which comprises at least 90% sequence identity to SEQ ID NO: 7, SEQ ID NO: 10; SEQ ID NO: 13 or SEQ ID NO: 16; a nucleotide sequence comprising a coding sequence which comprises at least 90% sequence identity to SEQ ID NO: 8, SEQ ID NO: 11; SEQ ID NO: 14 or SEQ ID NO: 17; and a nucleotide sequence encoding an amino acid sequence which comprises at least 90% sequence identity SEQ ID NO: 9, SEQ ID NO: 12; SEQ ID NO: 15 or SEQ ID NO: 18.

The Brassica plant may comprise four CKX3 genes, said Brassica plant selected from the group consisting of Brassica napus, Brassica juncea and Brassica carinata and may further comprise at least two mutant CKX3 alleles, or at least three mutant CKX3 alleles, or at least four mutant CKX3 alleles, or at least five mutant CKX3 alleles, or at least six mutant CKX3 alleles, or at least seven mutant CKX3 alleles, or at least eight mutant CKX3 alleles.

[23] In a particular embodiment the mutant CKX3 allele may be selected from a mutant CKX3 allele comprising a C to T substitution at a position corresponding to position 2244 of SEQ ID NO: 7 or position 1093 of SEQ ID No. 8; a mutant CKX3 allele comprising a C to T substitution at a position corresponding to position 2482 of SEQ ID NO: 10 or position 1168 of SEQ ID No. 11; a mutant CKX3 allele comprising a G to A substitution at a position corresponding to position 1893 of SEQ ID NO: 13 or position 876 of SEQ ID No. 14; or a mutant CKX3 allele comprising a C to T substitution at a position corresponding to position 2171 of SEQ ID NO: 16 or position 982 of SEQ ID No. 17.

The Brassica plant may be homozygous for the mutant CKX3 allele and/or for the mutant CKX5 allele.

Such plants may have increased flower number per plant, an increased pod number per plant, such as an increased pod or flower number on the main branch of the plant or an increased Thousand Seed Weight (TSW).

The invention also provides plant cells, pods, seeds, or progeny of the plant characterized by the presence of the mutant alleles herein described.

The invention further provides a mutant allele of a Brassica CKX3 or CKX5 gene, wherein the CKX5 gene is selected from the group consisting of: a nucleotide sequence which comprises at least 90% sequence identity to SEQ ID NO: 19 or SEQ ID NO: 23; (b) a nucleotide sequence comprising a coding sequence which comprises at least 90% sequence identity to SEQ ID NO: 20 or SEQ ID NO: 23; and a nucleotide sequence encoding an amino acid sequence which comprises at least 90% sequence identity to SEQ ID NO: 21, or SEQ ID NO: 24; and wherein the CKX3 gene is selected from the group consisting of a nucleotide sequence which comprises at least 90% sequence identity to SEQ ID NO: 7, SEQ ID NO: 10; SEQ ID NO: 13 or SEQ ID NO: 16; a nucleotide sequence comprising a coding sequence which comprises at least 90% sequence identity to SEQ ID NO: 8, SEQ ID NO: 11; SEQ ID NO: 14 or SEQ ID NO: 17; and a nucleotide sequence encoding an amino acid sequence which comprises at least 90% sequence identity SEQ ID NO: 9, SEQ ID NO: 12; SEQ ID NO: 15 or SEQ ID NO: 18.

In yet another embodiment, a mutant allele is provided selected from the group consisting of:
a. a mutant CKX5 allele comprising a G to A substitution at a position corresponding to position 465 of SEQ ID NO: 19 or position 465 pf SEQ ID No. 20;
b. a mutant CKX5 allele comprising a G to A substitution at a position corresponding to position 399 of SEQ ID NO: 19 or position 399 of SEQ ID No. 20; and
c. a mutant CKX5 allele comprising a G to A substitution at a position corresponding to position 465 of SEQ ID NO: 22 or position 399 of SEQ ID No. 23;
d. a mutant CKX3 allele comprising a C to T substitution at a position corresponding to position 2244 of SEQ ID NO: 7 or position 1093 of SEQ ID No. 8;
e. a mutant CKX3 allele comprising a C to T substitution at a position corresponding to position 2482 of SEQ ID NO: 10 or position 1168 of SEQ ID No. 11;
f. a mutant CKX3 allele comprising a G to A substitution at a position corresponding to position 1893 of SEQ ID NO: 13 or position 876 of SEQ ID No. 14;
g. a mutant CKX3 allele comprising a C to T substitution at a position corresponding to position 2171 of SEQ ID NO: 16 or position 982 of SEQ ID No. 17.

The invention also provides a chimeric gene comprising the following operably linked DNA fragments:
(a) a plant-expressible promoter;
(b) a DNA region, which when transcribed yields an RNA or protein molecule inhibitory to the expression or activity of one or more CKX5 or CKX5 and CKX3 genes or proteins; and optionally,
(c) a 3′ end region involved in transcription termination and polyadenylation.

Yet another embodiment of the invention concerns a method for identifying a mutant CKX5 or CKX3 allele as herein described in a biological sample, which comprises determining the presence of a mutant CKX5 or CKX3 specific region in a nucleic acid present in said biological sample.

Still another embodiment of the invention concerns a method for determining the zygosity status of a mutant CKX3 or CKX5 allele as herein described in a Brassica plant, plant material or seed, which comprises determining the presence of a mutant and/or a corresponding wild type CKX3 or CKX5 specific region in the genomic DNA of said plant, plant material or seed.

The invention also provides a kit for identifying a mutant CKX3 or CKX5 allele as herein described, in a biological sample, comprising a set of at least two primers, said set being selected from the group consisting of:
(a) a set of primers, wherein one of said primers specifically recognizes the 5′ or 3′ flanking region of the mutant allele and the other of said primers specifically recognizes the mutation region of the mutant CKX3 or CKX5 allele, and
(b) a set of primers, wherein one of said primers specifically recognizes the 5′ or 3′ flanking region of the mutant CKX3 or CKX5 allele and the other of said primers specifically recognizes the joining region between the 3′ or 5′ flanking region and the mutation region of the mutant CKX3 or CKX5 allele, respectively;
or said kit comprising a set of at least one probe, said probe being selected from the group consisting of:
(a) a probe specifically recognizing the mutation region of the mutant CKX3 or CKX5 allele, and
(b) a probe specifically recognizing the joining region between the 3′ or 5′ flanking region between the mutation region of the mutant CKX3 or CKX5 allele.

Also provided is a method for transferring at least one selected mutant CKX3 or CKX5 allele as herein described, from one plant to another plant comprising the steps of:
(a) identifying a first plant comprising at least one selected mutant CKX3 or CKX5 allele using the described method,
(b) crossing the first plant with a second plant not comprising the at least one selected mutant CKX3 or CKX5 allele and collecting F1 hybrid seeds from said cross,
(c) optionally, identifying F1 plants comprising the at least one selected mutant CKX3 or CKX5 allele using the described method,
(d) backcrossing the F1 plants comprising the at least one selected mutant CKX3 or CKX5 allele with the second plant not comprising the at least one selected mutant CKX3 or CKX5 allele for at least one generation (x) and collecting BCx seeds from said crosses, and
(e) identifying in every generation BCx plants comprising the at least one selected mutant CKX3 or CKX5 allele using the method according to the described method.

In yet another embodiment, the invention provides a method to increase flower number per plant, comprising introducing at least one mutant CKX5 or one mutant CKX5 and one mutant CKX3 allele into a Brassica plant; or introducing the chimeric gene herein described into a Brassica plant.

In yet another embodiment, the invention provides a method to increase pod number per plant, comprising introducing at least one mutant CKX5 or one mutant CKX5 and one mutant CKX3 allele into a Brassica plant; or introducing the chimeric gene herein described into a Brassica plant.

In yet another embodiment, the invention provides a method to increase TSW, comprising introducing at least one mutant CKX5 or one mutant CKX5 and one mutant CKX3 allele into a Brassica plant; or introducing the chimeric gene herein described into a Brassica plant.

In a particular embodiment, the invention provides a Brassica plant selected from the group consisting of:
a Brassica plant comprising a mutant CKX5 allele comprising a G to A substitution at a position corresponding to position 465 of SEQ ID NO: 19 or position 465 of SEQ ID No. 20, reference seeds comprising said allele having been deposited at the NCIMB Limited on 5 Oct. 2015, under accession number NCIMB 42464;
a Brassica plant comprising a mutant CKX5 allele comprising a G to A substitution at a position corresponding to position 399 of SEQ ID NO: 19 or position 399 of SEQ ID No. 20, reference seeds comprising said allele having been deposited at the NCIMB Limited on 5 Oct. 2015, under accession number NCIMB 42465;
a Brassica plant comprising a mutant CKX5 allele comprising a G to A substitution at a position corresponding to position 465 of SEQ ID NO: 22 or position 399 of SEQ ID No. 23, reference seeds comprising said allele having been deposited at the NCIMB Limited on 5 Oct. 2015, under accession number NCIMB 42464;
a Brassica plant comprising a mutant CKX3 allele comprising a C to T substitution at a position corresponding to position 2244 of SEQ ID NO: 7 or position 1093 of SEQ ID No. 8, reference seeds comprising said allele having been deposited at the NCIMB Limited on 5 Oct. 2015, under accession number NCIMB 42464;
a Brassica plant comprising a mutant CKX3 allele comprising a C to T substitution at a position corresponding to position 2482 of SEQ ID NO: 10 or position 1168 of SEQ ID No. 11, reference seeds comprising said allele having been deposited at the NCIMB Limited on 5 Oct. 2015, under accession number NCIMB 42464;
a Brassica plant comprising a mutant CKX3 allele comprising a G to A substitution at a position corresponding to position 1893 of SEQ ID NO: 13 or position 876 of SEQ ID No. 14, reference seeds comprising said allele having been deposited at the NCIMB Limited on 5 Oct. 2015, under accession number NCIMB 42464;
a Brassica plant comprising a mutant CKX3 allele comprising a C to T substitution at a position corresponding to position 2171 of SEQ ID NO: 16 or position 982 of SEQ ID No. 17, reference seeds comprising said allele having been deposited at the NCIMB Limited on 5 Oct. 2015, under accession number NCIMB 42464.

In still another embodiment, the invention provides the use of the mutant CKX5 or mutant CKX5 and mutant CKX3 alleles as herein described or the chimeric gene as herein described to increase flower number per plant, pod number per plant or increase TSW in Brassica plants or to produce oilseed rape oil or an oilseed rape seed cake.

### BRIEF DESCRIPTION OF THE DRAWINGS

FIG. 1: Alignment of amino acid sequences of proteins encoded by AtCKX3 from Arabidopsis thaliana (SEQ ID NO. 3); by BnCKX3-A1 wild type allele (SEQ ID NO. 9); by BnCKX3-A1 YIIN501 allele (SEQ ID NO. 25); by BnCKX3-A2 wild type allele (SEQ ID No. 12) and by BnCKX3-A2 YIIN502 allele (SEQ ID NO. 26). Boxes and arrows refer to the conserved motifs and sites as indicated in Table 1.

FIG. 2: Alignment of amino acid sequences of protein encoded by AtCKX3 from Arabidopsis thaliana (SEQ ID NO. 3); by BnCKX3-C1 wild type allele (SEQ ID NO. 15); by BnCKX3-C1 YIIN521 allele (SEQ ID NO. 27); by BnCKX3-C2 wild type allele (SEQ ID No. 18) or by BnCKX3-C1 YIIN531 allele (SEQ ID NO. 28). Boxes and arrows refer to the conserved motifs and sites as indicated in Table 1.

FIG. 3: Alignment of amino acid sequences of protein encoded by AtCKX5 from Arabidopsis thaliana (SEQ ID NO. 6); by BnCKX5-A1 wild type allele (SEQ ID NO. 21); by BnCKX5-A1 YIIN801 allele (SEQ ID NO. 29); by BnCKX5-A1 YIIN805 allele (SEQ ID NO. 30); by BnCKX3-C1 wild type allele (SEQ ID No. 24) or by BnCKX3-C1 YIIN811 allele (SEQ ID NO. 31). Boxes and arrows refer to the conserved motifs and sites as indicated in Table 2.

### GENERAL DEFINITIONS

A Brassica “fruit”, as used herein, refers to an organ of a Brassica plant that develops from a gynoecium composed of fused carpels, which, upon fertilization, grows to become a “(seed) pod” or “silique” that contains the developing seeds.

“Crop plant” refers to plant species cultivated as a crop, such as Brassica napus (AACC, 2n=38), Brassica juncea (AABB, 2n=36), Brassica carinata (BBCC, 2n=34), Brassica rapa (syn. B. campestris) (AA, 2n=20), Brassica oleracea (CC, 2n=18) or Brassica nigra (BB, 2n=16). The definition does not encompass weeds, such as Arabidopsis thaliana.

A “Brassica plant” as used herein refers to allotetraploid or amphidiploid Brassica napus (AACC, 2n=38), Brassica juncea (AABB, 2n=36), Brassica carinata (BBCC, 2n=34), or to diploid Brassica rapa (syn. B. campestris) (AA, 2n=20), Brassica oleracea (CC, 2n=18) or Brassica nigra (BB, 2n=16).

A “Crop of oilseed rape” as used herein refers to oilseed rape cultivated as a crop, such as Brassica napus, Brassica juncea, Brassica carinata, Brassica rapa (syn. B. campestris), Brassica oleracea or Brassica nigra.

The term “nucleic acid sequence” (or nucleic acid molecule) refers to a DNA or RNA molecule in single or double stranded form, particularly a DNA encoding a protein or protein fragment according to the invention. An “endogenous nucleic acid sequence” refers to a nucleic acid sequence which occurs naturally within a plant cell, e.g. an endogenous allele of a CKX3 or CKX5 gene present within the nuclear genome of a Brassica cell. An “isolated nucleic acid sequence” is used to refer to a nucleic acid sequence that is no longer in its natural environment, for example in vitro or in a recombinant bacterial or plant host cell.

The term “gene” means a DNA sequence comprising a region (transcribed region), which is transcribed into an RNA molecule (e.g. into a pre-mRNA, comprising intron sequences, which is then spliced into a mature mRNA, or directly into a mRNA without intron sequences) in a cell, operable linked to regulatory regions (e.g. a promoter). A gene may thus comprise several operably linked sequences, such as a promoter, a 5′ leader sequence comprising e.g. sequences involved in translation initiation, a (protein) coding region (cDNA or genomic DNA) and a 3′ non-translated sequence comprising e.g. transcription termination sites. “Endogenous gene” is used to differentiate from a “foreign gene”, “transgene” or “chimeric gene”, and refers to a gene from a plant of a certain plant genus, species or variety, which has not been introduced into that plant by transformation (i.e. it is not a “transgene”), but which is normally present in plants of that genus, species or variety, or which is introduced in that plant from plants of another plant genus, species or variety, in which it is normally present, by normal breeding techniques or by somatic hybridization, e.g., by protoplast fusion. Similarly, an “endogenous allele” of a gene is not introduced into a plant or plant tissue by plant transformation, but is, for example, generated by plant mutagenesis and/or selection or obtained by screening natural populations of plants, or by gene targeting.

“Expression of a gene” or “gene expression” refers to the process wherein a DNA region, which is operably linked to appropriate regulatory regions, particularly a promoter, is transcribed into an RNA molecule. The RNA molecule is then processed further (by post-transcriptional processes) within the cell, e.g. by RNA splicing and translation initiation and translation into an amino acid chain (protein), and translation termination by translation stop codons. The term “functionally expressed” is used herein to indicate that a functional protein is produced; the term “not functionally expressed” to indicate that a protein with significantly reduced or no functionality (biological activity) is produced or that no protein is produced (see further below).

The term “protein” refers to a molecule consisting of a chain of amino acids, without reference to a specific mode of action, size, 3-dimensional structure or origin. A “fragment” or “portion” of a CKX3 or CK5 protein may thus still be referred to as a “protein”. An “isolated protein” is used to refer to a protein that is no longer in its natural environment, for example in vitro or in a recombinant bacterial or plant host cell. “Amino acids” are the principal building blocks of proteins and enzymes. They are incorporated into proteins by transfer RNA according to the genetic code while messenger RNA is being decoded by ribosomes. During and after the final assembly of a protein, the amino acid content dictates the spatial and biochemical properties of the protein or enzyme. The amino acid backbone determines the primary sequence of a protein, but the nature of the side chains determines the protein's properties. “Similar amino acids”, as used herein, refers to amino acids that have similar amino acid side chains, i.e. amino acids that have polar, non-polar or practically neutral side chains “Non-similar amino acids”, as used herein, refers to amino acids that have different amino acid side chains, for example an amino acid with a polar side chain is non-similar to an amino acid with a non-polar side chain. Polar side chains usually tend to be present on the surface of a protein where they can interact with the aqueous environment found in cells (“hydrophilic” amino acids). On the other hand, “non-polar” amino acids tend to reside within the center of the protein where they can interact with similar non-polar neighbors (“hydrophobic” amino acids”). Examples of amino acids that have polar side chains are arginine, asparagine, aspartate, cysteine, glutamine, glutamate, histidine, lysine, serine, and threonine (all hydrophilic, except for cysteine which is hydrophobic). Examples of amino acids that have non-polar side chains are alanine, glycine, isoleucine, leucine, methionine, phenylalanine, proline, and tryptophan (all hydrophobic, except for glycine which is neutral).

The term “CKX gene” refers herein to a nucleic acid sequence encoding a cytokinin oxidase/dehydrogenase (CKX) protein, which is an enzyme (EC1.5.99.12 and EC1.4.3.18 that oxidatively degrades cytokinin. For example, the breakdown of the active cytokinin isopentenyladenine yields adenine and an unsaturated aldehyde, 3-methyl-2-butenal. CKX enzymes are FAD-dependent oxidases.

As used herein, the term “allele(s)” means any of one or more alternative forms of a gene at a particular locus. In a diploid (or amphidiploid) cell of an organism, alleles of a given gene are located at a specific location or locus (loci plural) on a chromosome. One allele is present on each chromosome of the pair of homologous chromosomes.

As used herein, the term “homologous chromosomes” means chromosomes that contain information for the same biological features and contain the same genes at the same loci but possibly different alleles of those genes. Homologous chromosomes are chromosomes that pair during meiosis. “Non-homologous chromosomes”, representing all the biological features of an organism, form a set, and the number of sets in a cell is called ploidy. Diploid organisms contain two sets of non-homologous chromosomes, wherein each homologous chromosome is inherited from a different parent. In amphidiploid species, essentially two sets of diploid genomes exist, whereby the chromosomes of the two genomes are referred to as “homeologous chromosomes” (and similarly, the loci or genes of the two genomes are referred to as homeologous loci or genes). A diploid, or amphidiploid, plant species may comprise a large number of different alleles at a particular locus.

As used herein, the term “heterozygous” means a genetic condition existing when two different alleles reside at a specific locus, but are positioned individually on corresponding pairs of homologous chromosomes in the cell. Conversely, as used herein, the term “homozygous” means a genetic condition existing when two identical alleles reside at a specific locus, but are positioned individually on corresponding pairs of homologous chromosomes in the cell.

As used herein, the term “locus” (loci plural) means a specific place or places or a site on a chromosome where for example a gene or genetic marker is found. For example, the “CKX3-A1 locus” refers to the position on a chromosome of the A genome where the CKX3-A1 gene (and two CKX3-A1 alleles) may be found; the “CKX3-A2 locus” refers to the position on a chromosome of the A genome where the CKX3-A2 gene (and two CKX-A2 alleles) may be found, while the“CKX3-C1 locus” refers to the position on a chromosome of the C genome where the CKX3-C1 gene (and two CKX3-C1 alleles) may be found, and the“CKX3-C2 locus” refers to the position on a chromosome of the C genome where the CKX3-C2 gene (and two CKX3-C2 alleles) may be found. Likewise, the “CKX5-A1 locus” refers to the position on a chromosome of the A genome where the CKX5-A1 gene (and two CKX5-A1 alleles) may be found, while the“CKX5-C1 locus” refers to the position on a chromosome of the C genome where the CKX5-C1 gene (and two CKX5-C1 alleles) may be found.

Whenever reference to a “plant” or “plants” according to the invention is made, it is understood that also plant parts (cells, tissues or organs, seed pods, seeds, severed parts such as roots, leaves, flowers, pollen, etc.), progeny of the plants which retain the distinguishing characteristics of the parents (especially the fruit dehiscence properties), such as seed obtained by selfing or crossing, e.g. hybrid seed (obtained by crossing two inbred parental lines), hybrid plants and plant parts derived there from are encompassed herein, unless otherwise indicated.

A “molecular assay” (or test) refers herein to an assay that determines (directly or indirectly) the presence or absence of one or more particular CKX3 or CKX5 alleles at one or more CKX3 or CKX5 loci (e.g., for Brassica napus, at one or more of the CKX3 -A1, CKX3-A2, CKX3 -C1, CKX3-C2, CKX5-A1 or CKX5-C1 loci). In one embodiment it allows one to determine whether a particular (wild type or induced variant) CKX3 and/or CKX5 allele is homozygous or heterozygous at the locus in any individual plant.

“Wild type” (also written “wildtype” or “wild-type”), as used herein, refers to a typical form of a plant or a gene as it most commonly occurs in nature. A “wild type plant” refers to a plant with the most common phenotype of such plant in the natural population. A “wild type allele” refers to an allele of a gene required to produce the wild-type phenotype. By contrast, an “induced variant plant” (or “mutant plant”) refers to a plant with a different phenotype of such plant in the natural population or produced by human intervention, e.g. by mutagenesis, and an “induced variant allele” (or a “mutant allele”) refers to an allele of a gene required to produce the variant (or mutant) phenotype.

As used herein, the term “wild type CKX3”, means a naturally occurring CKX3 allele found within Brassicaceae plants, especially Brassica plants, which encodes a functional CKX3 protein. As used herein, the term “wild type CKX5”, means a naturally occurring CKX5 allele found within Brassicaceae plants, especially Brassica plants, which encodes a functional CKX5 protein.

In contrast, the term “variant CKX3” (or “induced variant CKX3” or “mutant CKX3”), as used herein, refers to a CKX3 allele, which does not encode a functional CKX3 protein, i.e. a CKX3 allele encoding a non-functional CKX3 protein, which, as used herein, refers to a CKX3 protein having no biological activity or a significantly reduced biological activity as compared to the corresponding wild-type functional CKX3 protein, or encoding no CKX3 protein at all. Such a “mutant CKX3 allele” (also called “full knock-out” or “null” allele) is a wild-type CKX3 allele, which comprises one or more mutations in its nucleic acid sequence, whereby the mutation(s) preferably result in a significantly reduced (absolute or relative) amount of functional CKX3 protein in the cell in vivo. As used herein, a “full knock-out CKX3 allele” is a mutant CKX3 allele, the presence of which results at least in the increase of the number of flowers and/or pods on that plant, particularly on the main branch of that plant (potentially in combination with another CKX allele such as a mutant CKX5 allele). Likewise, the term “variant CKX5” (or “induced variant CKX5” or “mutant CKX5”), as used herein, refers to a CKX5 allele, which does not encode a functional CKX5 protein, i.e. a CKX5 allele encoding a non-functional CKX5 protein, which, as used herein, refers to a CKX5 protein having no biological activity or a significantly reduced biological activity as compared to the corresponding wild-type functional CKX5 protein, or encoding no CKX5 protein at all. Such a “mutant CKX5 allele” (also called “full knock-out” or “null” allele) is a wild type CKX5 allele, which comprises one or more mutations in its nucleic acid sequence, whereby the mutation(s) preferably result in a significantly reduced (absolute or relative) amount of functional CKX5 protein in the cell in vivo. As used herein, a “full knock-out CKX5 allele” is a mutant CKX5 allele, the presence of which results at least in the increase of the number of flowers and/or pods on that plant, particularly on the main branch of that plant (potentially in combination with another CKX allele such as a mutant CKX3 allele).

Mutant alleles of the CKX3 protein-encoding nucleic acid sequences are designated as “ckx3” (e.g., for Brassica napus, ckx3-a1, ckx3-a2, ckx3-c1 or ckx3-c2, respectively) herein. Mutant alleles of the CKX5 protein-encoding nucleic acid sequences are designated as “ckx5” (e.g., for Brassica napus, ckx5-a1 or ckx5-c1, respectively). Mutant alleles can be either “natural mutant” alleles, which are mutant alleles found in nature (e.g. produced spontaneously without human application of mutagens) or “induced mutant” alleles, which are induced by human intervention, e.g. by mutagenesis.

A “full knock-out mutant CKX3 allele” is, for example, a wild type CKX3 allele, which comprises one or more mutations in its nucleic acid sequence, for example, one or more non-sense or mis-sense mutations. In particular, such a full knock-out mutant CKX3 allele is a wild-type CKX3 allele, which comprises a mutation that preferably results in the production of a CKX3 protein or truncated CKX3 protein lacking at least one conserved motif, such as the signal peptide comprising the amino acid residues at positions corresponding to positions 1-31 of AtCKX3 (SEQ ID NO: 3); the FAD-binding region comprising residues at positions corresponding to positions of 66 to 243 of AtCKX3 (SEQ ID NO: 3); the FAD-binding amino acid residues comprising amino acids at positions corresponding to positions 100 to 104 of AtCKX3 (SEQ ID NO: 3); the FAD-binding amino acid residues comprising amino acids at positions corresponding to positions 105 to 106 of AtCKX3 (SEQ ID NO: 3); the FAD-binding histidine at a position corresponding to position 105 of AtCKX3(SEQ ID No. 3); the FAD-binding amino acid at a position corresponding to position 110 of AtCKX3 (SEQ ID No. 3); the FAD-binding amino acid at a position corresponding to position 167 of AtCKX3 (SEQ ID No. 3); the FAD-binding amino acid at a position corresponding to position 172 of AtCKX3 (SEQ ID No. 3); the FAD-binding amino acids at a position corresponding to positions 178 to 182 of AtCKX3 (SEQ ID No. 3); the FAD-binding amino acid at a position corresponding to position 233 of AtCKX3 (SEQ ID No. 3); the FAD-binding amino acid at a position corresponding to position 476 of AtCKX3 (SEQ ID No. 3); the cytokinin-binding amino acids at positions 244 to 517 of AtCKX3 (SEQ ID No.3); the GIWeVPHPWLNL motif at positions corresponding to positions 374 to 385 of AtCKX3 (SEQ ID No. 3) or the PGQxIF motif at positions corresponding to positions 512 to 517 of AtCKX3 (SEQ ID No. 3), such that the biological activity of the CKX3 protein is reduced or completely abolished, or whereby the mutation(s) preferably result in a significantly reduced amount of functional CKX3 protein, or no production of CKX3 protein. The latter may be accomplished by deletions removing the complete CKX3 encoding nucleotide sequence, or by deletions encompassing the 5′ end of the CKX3 coding region.

A “full knock-out mutant CKX5 allele” is, for example, a wild-type CKX5 allele, which comprises one or more mutations in its nucleic acid sequence, for example, one or more non-sense or mis-sense mutations. In particular, such a full knock-out mutant CKX5 allele is a wild-type CKX5 allele, which comprises a mutation that preferably results in the production of a CKX5 protein or truncated CKX5 protein lacking at least one conserved motif, such as the signal peptide comprising the amino acid residues at positions corresponding to positions 1-24 of AtCKX5 (SEQ ID NO: 6); the FAD-binding region comprising residues at positions corresponding to positions of 63 to 241 of AtCKX5 (SEQ ID NO: 6); the FAD-binding amino acid residues comprising amino acids at positions corresponding to positions 97 to 101 of AtCKX5 (SEQ ID NO: 6); the FAD-binding amino acid residues comprising amino acids at positions corresponding to positions 102 to 103 of AtCKX5 (SEQ ID NO: 6); the FAD-binding histidine at a position corresponding to position 102 of AtCKX5 (SEQ ID No. 6); the FAD-binding amino acid at a position corresponding to position 107 of AtCKX5; the FAD-binding amino acid at a position corresponding to position 165 of AtCKX5 (SEQ ID No. 6); the FAD-binding amino acid at a position corresponding to position 170 of AtCKX3 (SEQ ID No. 6); the FAD-binding amino acids at a position corresponding to positions 176 to 180 of AtCKX5 (SEQ ID No. 6); the FAD-binding amino acid at a position corresponding to position 231 of AtCKX5 (SEQ ID No. 6); the FAD-binding amino acid at a position corresponding to position 479 of AtCKX5 (SEQ ID No. 6); the cytokinin-binding amino acids at positions 242 to 520 of AtCKX5 (SEQ ID No.6); the GIWeVPHPWLNL motif at positions corresponding to positions 374 to 385 of AtCKX5 (SEQ ID No. 6) or the PGQxIF motif at positions corresponding to positions 515 to 520 of AtCKX5 (SEQ ID No.6), such that the biological activity of the CKX5 protein is reduced or completely abolished, or whereby the mutation(s) preferably result in a significantly reduced amount of functional CKX5 protein, or no production of CKX5 protein. The latter may be accomplished by deletions removing the complete CKX5 encoding nucleotide sequence, or by deletions encompassing the 5′ end of the CKX5 coding region.

A “corresponding position” or “a position corresponding to position” in accordance with the present invention it is to be understood that nucleotides/amino acids may differ in the indicated number but may still have similar neighbouring nucleotides/amino acids. Said nucleotides/amino acids which may be exchanged, deleted or added are also comprised by the term “corresponding position”. The reference sequence may be the AtCKX3 or AtCKX5 sequence from Arabidopsis thaliana. Tables of correspondence between the reference amino acid sequences of CKX3 and CKX5 proteins from Arabidopsis thaliana with exemplary amino acid sequences of CKX3 and CKX5 proteins from Brassica napus are provided in

Tables 1 and 2.

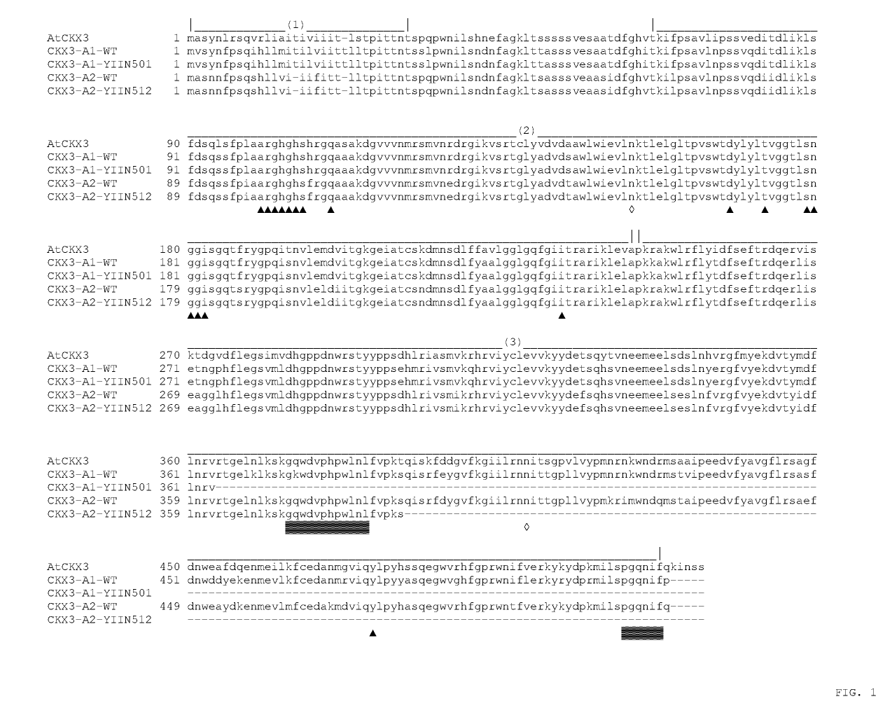

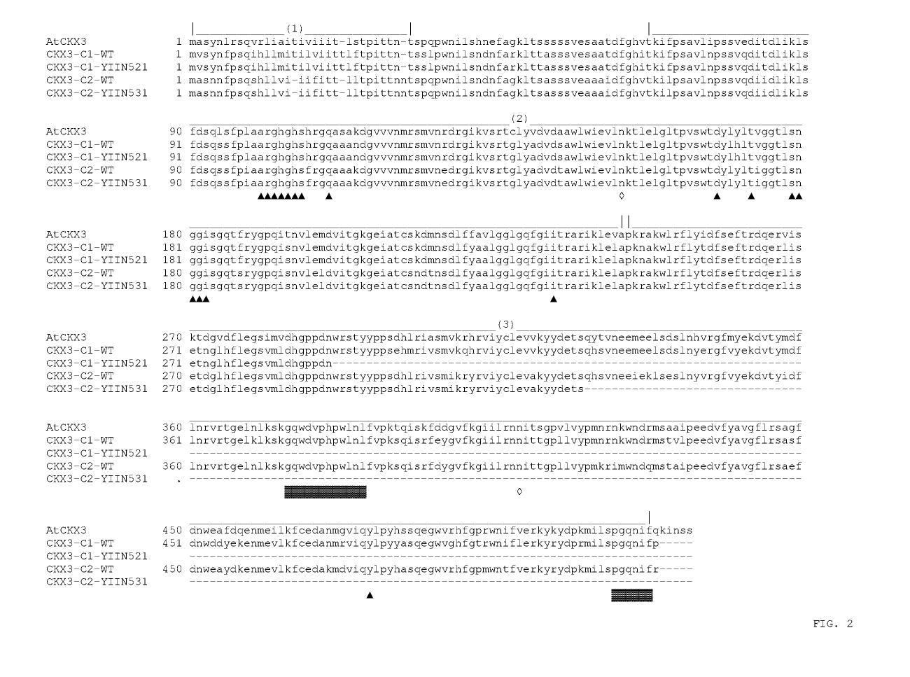

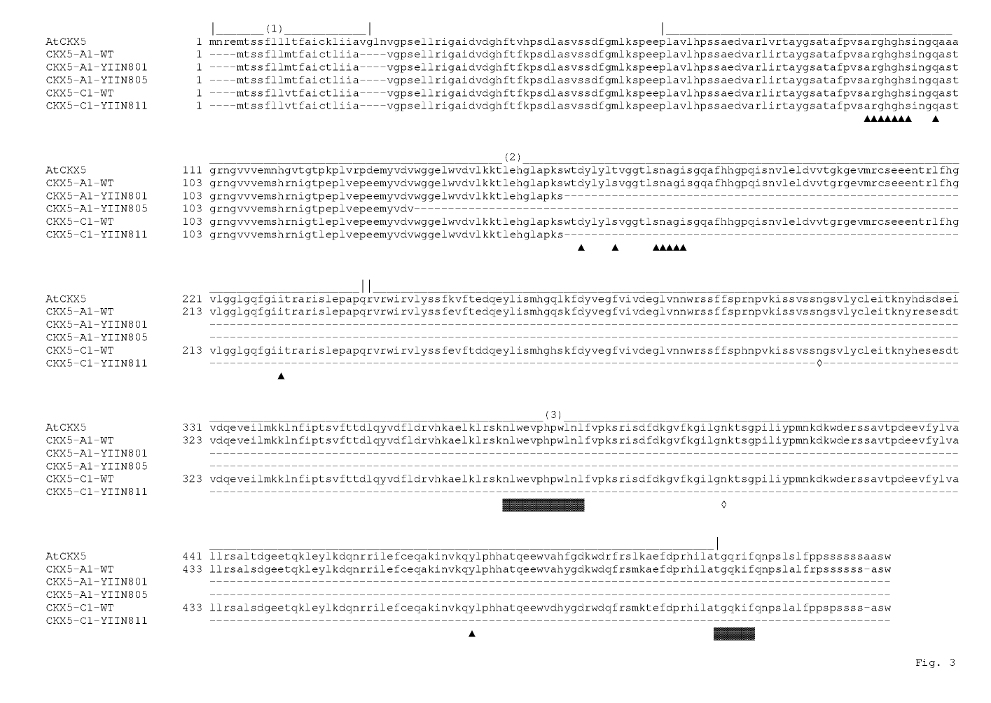

In order to determine whether a nucleotide residue or amino acid residue in a given CKX3 or CKX5 nucleotide/amino acid sequence corresponds to a certain position in the nucleotide sequence of another CKX3 or CKX5 nucleotide or amino acid sequence, the skilled person can use means and methods well-known in the art, e.g., alignments, either manually or by using computer programs such as BLAST (Altschul et al. (1990), Journal of Molecular Biology, 215, 403-410), which stands for Basic Local Alignment Search Tool or ClustalW (Thompson et al. (1994), Nucleic Acid Res., 22, 4673-4680) or any other suitable program which is suitable to generate sequence alignments. For an alignment of the Arabidopsis and Brassica CKX3 or CKX5 amino acid sequences, see for example, FIGS. 1 to 3.

A “significantly reduced amount of functional CKX3 or CKX5 protein” refers to a reduction in the amount of a functional CKX3 or CKX5 protein, respectively, produced by the cell comprising a mutant CKX allele by at least 30%, 40%, 50%, 60%, 70%, 80%, 90%, 95% or 100% (i.e. no functional CKX3 or CKX5 protein is produced by the cell) as compared to the amount of the functional protein produced by the cell not comprising the mutant CKX3 or CKX5 allele. This definition encompasses the production of a “non-functional” CKX3 or CKX5 protein (e.g. truncated CKX3 or CKX5 protein) having no biological activity in vivo, the reduction in the absolute amount of the functional CKX3 or CKX5 protein (e.g. no functional CKX3 or CKX5 protein being made due to the mutation in the CKX3 or CKX5 gene), the production of a CKX3 or CKX5 protein with significantly reduced biological activity compared to the activity of a functional wild type protein (such as a CKX3 or CKX5 protein in which one or more amino acid residues that are crucial for the biological activity of the encoded CKX3 or CKX5 protein, are substituted for another amino acid residue) and/or the adverse effect of dominant negative CKX3 or CKX5 proteins on other functional and/or partially functional CKX3 or CKX5 proteins.

The term “mutant CKX3 or CKX5protein”, as used herein, refers to a CKX3 or CKX5 protein encoded by a mutant CKX3 or CKX5 nucleic acid sequence (“ckx3 or ckx5 allele”) whereby the mutation results in a significantly reduced and/or no CKX3 or CKX5 activity in vivo, compared to the activity of the CKX3 or CKX5 protein encoded by a non-mutant, wild type CKX3 or CKX5 sequence (“CKX3 allele” respectively “CKX5 allele”).

“Mutagenesis” or “induced variation”, as used herein, refers to the process in which plant cells (e.g., a plurality of Brassica seeds or other parts, such as pollen, etc.) are subjected to a technique which induces mutations in the DNA of the cells, such as contact with a mutagenic agent, such as a chemical substance (such as ethylmethylsulfonate (EMS), ethylnitrosourea (ENU), etc.) or ionizing radiation (neutrons (such as in fast neutron mutagenesis, etc.), alpha rays, gamma rays (such as that supplied by a Cobalt 60 source), X-rays, UV-radiation, etc.), or a combination of two or more of these. Thus, the desired mutagenesis of one or more CKX alleles may be accomplished by use of chemical means such as by contact of one or more plant tissues with ethylmethylsulfonate (EMS), ethylnitrosourea, etc., by the use of physical means such as x-ray, etc, or by gamma radiation, such as that supplied by a Cobalt 60 source. While mutations created by irradiation are often large deletions or other gross lesions such as translocations or complex rearrangements, mutations created by chemical mutagens are often more discrete lesions such as point mutations. For example, EMS alkylates guanine bases, which results in base mispairing: an alkylated guanine will pair with a thymine base, resulting primarily in G/C to A/T transitions. Following mutagenesis, Brassica plants are regenerated from the treated cells using known techniques. For instance, the resulting Brassica seeds may be planted in accordance with conventional growing procedures and following self-pollination seed is formed on the plants. Alternatively, doubled haploid plantlets may be extracted to immediately form homozygous plants, for example as described by Coventry et al. (1988, Manual for Microspore Culture Technique for Brassica napus. Dep. Crop Sci. Techn. Bull. OAC Publication 0489. Univ. of Guelph, Guelph, Ontario, Canada). Additional seed that is formed as a result of such self-pollination in the present or a subsequent generation may be harvested and screened for the presence of mutant CKX alleles. Several techniques are known to screen for specific mutant alleles, e.g., Deleteagene™ (Delete-a-gene; Li et al., 2001, Plant J 27: 235-242) uses polymerase chain reaction (PCR) assays to screen for deletion mutants generated by fast neutron mutagenesis, TILLING (targeted induced local lesions in genomes; McCallum et al., 2000, Nat Biotechnol 18:455-457) identifies EMS-induced point mutations, etc. Additional techniques to screen for the presence of specific mutant CKX3 or CKX5 alleles are described in the Examples below. Mutagenesis can comprise random mutagenesis, or can comprise targeted mutagenesis. Mutagenesis can also result in epimutations that cause epigenetic silencing.

The term “gene targeting” refers herein to directed gene modification that uses mechanisms such as homologous recombination, mismatch repair or site-directed mutagenesis. The method can be used to replace, insert and delete endogenous sequences or sequences previously introduced in plant cells. Methods for gene targeting can be found in, for example, WO 2006/105946 or WO2009/002150.

As used herein, the term “non-naturally occurring” or “cultivated” when used in reference to a plant, means a plant with a genome that has been modified by man. A transgenic plant, for example, is a non-naturally occurring plant that contains an exogenous nucleic acid molecule, e.g., a chimeric gene comprising a transcribed region which when transcribed yields a biologically active RNA molecule capable of reducing the expression of an endogenous gene, such as a CKX3 or CKX5 gene, and, therefore, has been genetically modified by man. In addition, a plant that contains a mutation in an endogenous gene, for example, a mutation in an endogenous CKX3 or CKX5 gene, (e.g. in a regulatory element or in the coding sequence) as a result of an exposure to a mutagenic agent is also considered a non-naturally plant, since it has been genetically modified by man. Furthermore, a plant of a particular species, such as Brassica napus, that contains a mutation in an endogenous gene, for example, in an endogenous CKX3 or CKX5 gene, that in nature does not occur in that particular plant species, as a result of, for example, directed breeding processes, such as marker-assisted breeding and selection or introgression, with a plant of the same or another species, such as Brassica juncea or rapa, of that plant is also considered a non-naturally occurring plant. In contrast, a plant containing only spontaneous or naturally occurring mutations, i.e. a plant that has not been genetically modified by man, is not a “non-naturally occurring plant” as defined herein and, therefore, is not encompassed within the invention. One skilled in the art understands that, while a non-naturally occurring plant typically has a nucleotide sequence that is altered as compared to a naturally occurring plant, a non-naturally occurring plant also can be genetically modified by man without altering its nucleotide sequence, for example, by modifying its methylation pattern.

The term “ortholog” of a gene or protein refers herein to the homologous gene or protein found in another species, which has the same function as the gene or protein, but is (usually) diverged in sequence from the time point on when the species harboring the genes diverged (i.e. the genes evolved from a common ancestor by speciation). Orthologs of the Brassica napus CKX3 or CKX5 genes may thus be identified in other plant species (e.g. other pod-bearing plant species, such as other Brassicaceae plants, or Fabaceae plants such as, for example, Phaseolus species, or soybeans (Glycine max)) based on both sequence comparisons (e.g. based on percentages sequence identity over the entire sequence or over specific domains) and/or functional analysis.

A “variety” is used herein in conformity with the UPOV convention and refers to a plant grouping within a single botanical taxon of the lowest known rank, which grouping can be defined by the expression of the characteristics resulting from a given genotype or combination of genotypes, can be distinguished from any other plant grouping by the expression of at least one of the said characteristics and is considered as a unit with regard to its suitability for being propagated unchanged (stable).

The term “comprising” is to be interpreted as specifying the presence of the stated parts, steps or components, but does not exclude the presence of one or more additional parts, steps or components. A plant comprising a certain trait may thus comprise additional traits.

It is understood that when referring to a word in the singular (e.g. plant or root), the plural is also included herein (e.g. a plurality of plants, a plurality of roots). Thus, reference to an element by the indefinite article “a” or “an” does not exclude the possibility that more than one of the element is present, unless the context clearly requires that there be one and only one of the elements. The indefinite article “a” or “an” thus usually means “at least one”.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other, is regarded as a position with non-identical residues. The “optimal alignment” of two sequences is found by aligning the two sequences over the entire length according to the Needleman and Wunsch global alignment algorithm (Needleman and Wunsch, 1970, J Mol Biol 48(3):443-53) in The European Molecular Biology Open

Software Suite (EMBOSS, Rice et al., 2000, Trends in Genetics 16(6): 276-277; see e.g. http://www.ebi.ac.uk/emboss/align/index.html) using default settings (gap opening penalty =10 (for nucleotides)/10 (for proteins) and gap extension penalty=0.5 (for nucleotides)/0.5 (for proteins)). For nucleotides the default scoring matrix used is EDNAFULL and for proteins the default scoring matrix is EBLOSUM62.

“Substantially identical” or “essentially similar”, as used herein, refers to sequences, which, when optimally aligned as defined above, share at least a certain minimal percentage of sequence identity (as defined further below).

“Stringent hybridization conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tₘ) for the specific sequences at a defined ionic strength and pH. The Tₘ is the temperature (under defined ionic strength and pH) at which 50% of the target sequence hybridizes to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridizations (Northern blots using a probe of e.g. 100 nt) are for example those which include at least one wash in 0.2×SSC at 63° C. for 20 min, or equivalent conditions.

“High stringency conditions” can be provided, for example, by hybridization at 65° C. in an aqueous solution containing 6×SSC (20×SSC contains 3.0 M NaCl, 0.3 M Na-citrate, pH 7.0), 5× Denhardt's (100× Denhardt's contains 2% Ficoll, 2% Polyvinyl pyrollidone, 2% Bovine Serum Albumin), 0.5% sodium dodecyl sulphate (SDS), and 20 μg/ml denaturated carrier DNA (single-stranded fish sperm DNA, with an average length of 120-3000 nucleotides) as non-specific competitor. Following hybridization, high stringency washing may be done in several steps, with a final wash (about 30 min) at the hybridization temperature in 0.2-0.1×SSC, 0.1% SDS.

“Moderate stringency conditions” refers to conditions equivalent to hybridization in the above described solution but at about 60-62° C. Moderate stringency washing may be done at the hybridization temperature in 1×SSC, 0.1% SDS.

“Low stringency” refers to conditions equivalent to hybridization in the above described solution at about 50-52° C. Low stringency washing may be done at the hybridization temperature in 2×SSC, 0.1% SDS. See also Sambrook et al. (1989) and Sambrook and Russell (2001).

“Increased yield” or “increased harvested yield” or “increased seed or grain yield” refers to the larger amount of seeds or grains harvested from a plurality of plants, each comprising mutant CKX5 or CKX3/CKX5 alleles according to the invention, when compared to the amount of seeds or grains harvested from a similar number of isogenic plants without the mutant CKX5 or CKX3/CKX5 alleles. Yield is typically expressed in volume units of harvested seeds or grains per surface units, such as bushels/acre or kg/ha (although other units may be used such as gram/test plot or even grams/plant). The yield increase is typically expressed in percentage, whereby the yield of the reference or control plant is referred to as 100% and the yield of the plants according to the inventions is expressed in % relative to the yield of the control plant. Yield increase may be a yield of at least 101%, of at least 102%, of at least 103%, of at least 105%, of at least 108%, of at least 110%.

“Thousand Seed Weight” (TSW) refers to the weight in grams of 1000 seeds or grains . Increased Thousand Seed Weight” refers to the larger weight of 1000 seeds harvested from plants comprising mutant CKX5 or CKX3/CKX5 alleles according to the invention, when compared to the weight of 1000 seeds or grains harvested from a similar number of isogenic plants without the mutant CKX5 or CKX3/CKX5 alleles.

“Increased number of flowers” or “increased number of flowers on the main branch” refers to the larger amount of flowers on plants, respectively larger amount of flowers on the main branch of a plant, comprising mutant CKX5 or CKX3/CKX5 alleles according to the invention, when compared to the amount of flowers on plants, respectively larger amount of flowers on the main branch of plants, preferably isogenic plants without the mutant CKX5 or CKX3/CKX5 alleles.

“Increased number of pods” or “increased number of pods on the main branch” refers to the larger amount of pods on plants, respectively larger amount of pods on the main branch of plants, comprising mutant CKX5 or CKX3/CKX5 alleles according to the invention, when compared to the amount of pods on plants, respectively larger amount of pods on the main branch of plants (such as isogenic plants) without the mutant CKX5 or CKX3/CKX5 alleles.

### DETAILED DESCRIPTION

Brassica napus (genome AACC, 2n=4x=38), which is an allotetraploid (amphidiploid) species containing essentially two diploid genomes (the A and the C genome) due to its origin from diploid ancestors. Brassica napus comprises four CKX3 genes in its genome; two CKX3 genes are located on the A genome (hereinafter called CKX3-A1 and CKX3-A2) and two CKX3 genes are located on the C genome, herein after called CKX3-C1 and CKX3-C2. Brassica napus also comprises two CKX5 genes in its genome; one CKX5 gene is located on the A genome (hereinafter called CKX5-A1) and one CKX5 gene is located on the C genome, herein after called CKX5-C1. It was found by the inventors that the presence of mutant alleles of the CKX5 or mutant alleles of CKX5 and CKX3 increases the number of flowers per plant, particularly the number of flowers on the main branch. Also the number of pods on the main branch can be increased, as well as the number of seeds per pod on the main branch. Furthermore an increase in Thousand Seed Weight (TSW) can be achieved, particularly a higher TSW without a significant negative effect on seed yield.

The application relates to Brassica plants in which expression of CKX5 or CKX5 and CKX3 is functionally reduced. Functionally reduced expression can be reduction in CKX3/CKX5 protein production and/or activity.

Thus, in a first aspect, a Brassica plant is provided comprising at least one, preferably two CKX5 genes, characterised in that it comprises at least one mutant CKX5 allele in its genome. In a further aspect, a Brassica plant is provided comprising at least one CKX5 and at least two CKX3 genes, preferably two CKX5 genes and four CKX3 genes, characterised in that it comprises at least one mutant CKX5 allele and one mutant CKX3 in its genome.

In a further aspect, the mutant CKX3 allele is a mutant allele of a CKX3 gene comprising a nucleic acid sequence selected from the group consisting of:
a nucleotide sequence which comprises at least 90% sequence identity to SEQ ID NO: 7, SEQ ID NO: 10; SEQ ID NO: 13 or SEQ ID NO: 16;
a nucleotide sequence comprising a coding sequence which comprises at least 90% sequence identity to SEQ ID NO: 8, SEQ ID NO: 11; SEQ ID NO: 14 or SEQ ID NO: 17; and
a nucleotide sequence encoding an amino acid sequence which comprises at least 90% sequence identity SEQ ID NO: 9, SEQ ID NO: 12; SEQ ID NO: 15 or SEQ ID NO: 18; and
the mutant CKX5 allele is a mutant allele of a CKX gene comprising a nucleic acid sequence selected from the group consisting of:
a nucleotide sequence which comprises at least 90% sequence identity to SEQ ID NO: 19 or SEQ ID NO: 23;
a nucleotide sequence comprising a coding sequence which comprises at least 90% sequence identity to SEQ ID NO: 20 or SEQ ID NO: 23; and
a nucleotide sequence encoding an amino acid sequence which comprises at least 90% sequence identity to SEQ ID NO: 21, or SEQ ID NO: 24.

In a further aspect, the plant according to the invention is a Brassica plant comprising two CKX5 and four CKX3 genes, said Brassica plant selected from the group consisting of Brassica napus, Brassica juncea and Brassica carinata. In another embodiment, the plant according to the invention comprises comprising at least two mutant CKX5 alleles, or at least three mutant CKX5 alleles or four mutant CKX5 alleles, or at least two, three or four mutant CKX5 alleles and three mutant CKX3 alleles, or at least four mutant CKX3 alleles, or at least five mutant CKX3 alleles, or at least six mutant CKX3 alleles, or at least seven mutant CKX3 alleles, or at least eight mutant CKX3 alleles.

In yet another embodiment, the plants according to the invention comprise a mutant CKX5 allele selected from the group consisting of:
a mutant CKX5 allele comprising a G to A substitution at a position corresponding to position 465 of SEQ ID NO: 19 or position 465 pf SEQ ID No. 20;
a mutant CKX5 allele comprising a G to A substitution at a position corresponding to position 399 of SEQ ID NO: 19 or position 399 of SEQ ID No. 20;
a mutant CKX5 allele comprising a G to A substitution at a position corresponding to position 465 of SEQ ID NO: 22 or position 399 of SEQ ID No. 23;
   or the plants comprise a mutant CKX3 allele selected from the group consisting of:
a mutant CKX3 allele comprising a C to T substitution at a position corresponding to position 2244 of SEQ ID NO: 7 or position 1093 of SEQ ID No. 8;
a mutant CKX3 allele comprising a C to T substitution at a position corresponding to position 2482 of SEQ ID NO: 10 or position 1168 of SEQ ID No. 11;
a mutant CKX3 allele comprising a G to A substitution at a position corresponding to position 1893 of SEQ ID NO: 13 or position 876 of SEQ ID No. 14;
a mutant CKX3 allele comprising a C to T substitution at a position corresponding to position 2171 of SEQ ID NO: 16 or position 982 of SEQ ID No. 17.

In yet another embodiment, the plants according to the invention comprise at least one variant CKX5 protein wherein the variant CKX5 protein consist of an amino acid sequence selected from:
the amino acid sequence of SEQ ID No. 29;
the amino acid sequence of SEQ ID No. 30; or
the amino acid sequence of SEQ ID No. 31.

The plants according to the invention may in addition to the variant CKX5 protein comprise at least one variant CKX3 protein, wherein the variant CKX3 protein consist of an amino acid sequence selected from
the amino acid sequence of SEQ ID No. 25;
the amino acid sequence of SEQ ID No. 26;
the amino acid sequence of SEQ ID No. 27; or
the amino acid sequence of SEQ ID No. 28.

In again a further embodiment, said plant is homozygous for the mutant CKX5 allele or is homozygous for both the mutant CKX5 and mutant CK3 allele. In yet another embodiment, said plant has increased number of flowers per plant. In yet another embodiment, said plant has an increased number of pods per plant. In still another embodiment, said plant has an increased TSW.

A further embodiment provides a plant cell, pod, seed or progeny of the plant according to the invention.

In yet another embodiment, a Brassica plant is provided comprising wherein expression of at least one CKX5 gene or at least one CKX5 and at least one CKX3 gene is reduced. Expression can be reduced, for example, by introduction of a chimeric gene into said plant comprising a DNA region yielding an RNA molecule inhibitory to the expression of one or more CKX5 or CKX5 and CKX3 genes. In one embodiment, said plant comprises a chimeric gene, said chimeric gene comprising the following operably linked DNA fragments:
i. a plant-expressible promoter;
ii. a DNA region, which when transcribed yields an RNA or protein molecule inhibitory to the expression of one or more CKX5 or CKX5 and CKX3 genes encoding; and, optionally,
iii. a 3′ end region involved in transcription termination and polyadenylation.

Said DNA region may yield a sense RNA molecule capable of down-regulating expression of one or more CKX5 or CKX3 genes by co-suppression. The transcribed DNA region will yield upon transcription a so-called sense RNA molecule capable of reducing the expression of a CKX5 or CKX3 gene in the target plant or plant cell in a transcriptional or post-transcriptional manner. The transcribed DNA region (and resulting RNA molecule) comprises at least 19 or 20 consecutive nucleotides having at least 95% sequence identity, preferably are identical to a part of the nucleotide sequence of one or more CKX5 or CKX3 genes present in the plant cell or plant. The DNA region may thus comprise at least 19 or 20 consecutive nucleotides of the nucleotide sequence of SEQ ID Nos: 7, 8, 10, 11, 13, 14, 16 or 17 for CKX3 inhibitory RNA and/or SEQ ID Nos: 19, 20, 22 or 23 for CKX5 inhibitory RNA.

Said DNA region may also yield an antisense RNA molecule capable of down-regulating expression of one or more CKX5 or CKX3 genes. The transcribed DNA region will yield upon transcription a so-called antisense RNA molecule capable of reducing the expression of a CKX5 or CKX3 gene in the target plant or plant cell in a transcriptional or post-transcriptional manner. The transcribed DNA region (and resulting RNA molecule) comprises at least 20 consecutive nucleotides having at least 95% sequence identity to the complement of the nucleotide sequence of one or more functional CKX5 or CKX3 genes present in the plant cell or plant. The DNA region may thus comprise at least 19 or 20 consecutive nucleotides of the complement of the nucleotide sequence of SEQ ID Nos: 7, 8, 10, 11, 13, 14, 16 or 17 for CKX3 inhibitory RNA and/or SEQ ID Nos: 19, 20, 22 or 23 for CKX5 inhibitory RNA.

The minimum nucleotide sequence of the antisense or sense RNA region of about 20 nt of the CKX5 or CKX3 gene may be comprised within a larger RNA molecule, varying in size from 20 nt to a length equal to the size of the target gene. The mentioned antisense or sense nucleotide regions may thus be about from about 21 nt to about 1300 nt long, such as 21 nt, 40 nt, 50 nt, 100 nt, 200 nt, 300 nt, 500 nt, 1000 nt, or even about 1300 nt or larger in length. Moreover, it is not required for the purpose of the invention that the nucleotide sequence of the used inhibitory CKX5 or CKX3 RNA molecule or the encoding region of the transgene, is completely identical or complementary to the endogenous CKX5 or CKX3 gene the expression of which is targeted to be reduced in the plant cell. The longer the sequence, the less stringent the requirement for the overall sequence identity is. Thus, the sense or antisense regions may have an overall sequence identity of about 40% or 50% or 60% or 70% or 80% or 90% or 100% to the nucleotide sequence of the endogenous CKX5 or CKX3 gene or the complement thereof. However, as mentioned, antisense or sense regions should comprise a nucleotide sequence of 20 consecutive nucleotides having about 95 to about 100% sequence identity to the nucleotide sequence of the endogenous CKX5 or CKX3 gene. The stretch of about 95 to about 100% sequence identity may be about 50, 75 or 100 nt. It will be clear that all combinations between mentioned length and sequence identity can be made, both in sense and/or antisense orientation.

The efficiency of the above mentioned chimeric genes for antisense RNA or sense RNA-mediated gene expression level down-regulation may be further enhanced by inclusion of DNA elements which result in the expression of aberrant, non-polyadenylated CKX5 or CKX3 inhibitory RNA molecules. One such DNA element suitable for that purpose is a DNA region encoding a self-splicing ribozyme, as described in WO 00/01133. The efficiency may also be enhanced by providing the generated RNA molecules with nuclear localization or retention signals as described in WO 03/076619.

Said DNA region may also yield a double-stranded RNA molecule capable of down-regulating CKX5 or CKX3 gene expression. Upon transcription of the DNA region the RNA is able to form dsRNA molecule through conventional base paring between a sense and antisense region, whereby the sense and antisense region are nucleotide sequences as hereinbefore described. dsRNA-encoding CKX5 or CKX3 expression-reducing chimeric genes according to the invention may further comprise an intron, such as a heterologous intron, located e.g. in the spacer sequence between the sense and antisense RNA regions in accordance with the disclosure of WO 99/53050 (incorporated herein by reference). To achieve the construction of such a transgene, use can be made of the vectors described in WO 02/059294 A1.

Said DNA region may also yield a pre-miRNA molecule which is processed into a miRNA capable of guiding the cleavage of CKX5 or CKX3 mRNA. miRNAs are small endogenous RNAs that regulate gene expression in plants, but also in other eukaryotes. In plants, these about 21 nucleotide long RNAs are processed from the stem-loop regions of long endogenous pre-miRNAs by the cleavage activity of DICERLIKE1 (DCL1). Plant miRNAs are highly complementary to conserved target mRNAs, and guide the cleavage of their targets. miRNAs appear to be key components in regulating the gene expression of complex networks of pathways involved inter alia in development.

As used herein, a “miRNA” is an RNA molecule of about 20 to 22 nucleotides in length which can be loaded into a RISC complex and direct the cleavage of a target RNA molecule, wherein the target RNA molecule comprises a nucleotide sequence essentially complementary to the nucleotide sequence of the miRNA molecule whereby one or more of the following mismatches may occur:
A mismatch between the nucleotide at the 5′ end of said miRNA and the corresponding nucleotide sequence in the target RNA molecule;
A mismatch between any one of the nucleotides in position 1 to position 9 of said miRNA and the corresponding nucleotide sequence in the target RNA molecule;
Three mismatches between any one of the nucleotides in position 12 to position 21 of said miRNA and the corresponding nucleotide sequence in the target RNA molecule provided that there are no more than two consecutive mismatches;
No mismatch is allowed at positions 10 and 11 of the miRNA (all miRNA positions are indicated starting from the 5′ end of the miRNA molecule).

As used herein, a “pre-miRNA” molecule is an RNA molecule of about 100 to about 200 nucleotides, preferably about 100 to about 130 nucleotides which can adopt a secondary structure comprising a dsRNA stem and a single stranded RNA loop and further comprising the nucleotide sequence of the miRNA and its complement sequence of the miRNA* in the double-stranded RNA stem. Preferably, the miRNA and its complement are located about 10 to about 20 nucleotides from the free ends of the miRNA dsRNA stem. The length and sequence of the single stranded loop region are not critical and may vary considerably, e.g. between 30 and 50 nt in length. Preferably, the difference in free energy between unpaired and paired RNA structure is between −20 and −60 kcal/mole, particularly around −40 kcal/mole. The complementarity between the miRNA and the miRNA* do not need to be perfect and about 1 to 3 bulges of unpaired nucleotides can be tolerated. The secondary structure adopted by an RNA molecule can be predicted by computer algorithms conventional in the art such as mFold, UNAFold and RNAFold. The particular strand of the dsRNA stem from the pre-miRNA which is released by DCL activity and loaded onto the RISC complex is determined by the degree of complementarity at the 5′ end, whereby the strand which at its 5′ end is the least involved in hydrogen bounding between the nucleotides of the different strands of the cleaved dsRNA stem is loaded onto the RISC complex and will determine the sequence specificity of the target RNA molecule degradation. However, if empirically the miRNA molecule from a particular synthetic pre-miRNA molecule is not functional because the “wrong” strand is loaded on the RISC complex, it will be immediately evident that this problem can be solved by exchanging the position of the miRNA molecule and its complement on the respective strands of the dsRNA stem of the pre-miRNA molecule. As is known in the art, binding between A and U involving two hydrogen bounds, or G and U involving two hydrogen bounds is less strong that between G and C involving three hydrogen bounds.

miRNA molecules may be comprised within their naturally occurring pre-miRNA molecules but they can also be introduced into existing pre-miRNA molecule scaffolds by exchanging the nucleotide sequence of the miRNA molecule normally processed from such existing pre-miRNA molecule for the nucleotide sequence of another miRNA of interest. The scaffold of the pre-miRNA can also be completely synthetic. Likewise, synthetic miRNA molecules may be comprised within, and processed from, existing pre-miRNA molecule scaffolds or synthetic pre-miRNA scaffolds.

Also suitable to the invention is a Brassica plant comprising at least two CKX5 or CKX3 genes, wherein CKX5 or CKX3 protein activity is reduced, such as a Brassica plant comprising a DNA construct plant which encodes a dominant-negative CKX5 or CKX3 protein, or a DNA construct which encodes inactivating antibodies to CKX5 or CKX3 proteins, or a DNA construct encoding a protein which specifically inactivates the CKX5 or CKX3 protein, such as a protein with a specific CKX5 or CKX3 binding domain and a protein cleavage activity. “Inactivating antibodies to CKX5 or CKX3 proteins” are antibodies or parts thereof which specifically bind at least to some epitopes of CKX5 or CKX3 PGAZ proteins, and which inhibit the activity of the target protein. CKX5 or CKX3 protein activity can also be reduced, for example, by aggregating CKX5 or CKX3 proteins (see, e.g., WO2007/071789), or by scaffolding target proteins (see, e.g., WO2009/030780).

Said Brassica plant comprising at least two CKX5 or CKX3 genes, wherein expression of at least one CKX5 or CKX3 gene is reduced, can, for example, be a Brassica plant comprising four CKX5 or CKX3 genes, said Brassica plant selected from the group consisting of Brassica napus, Brassica juncea and Brassica carinata. In said Brassica plant, expression of at least one, or at least two, or at least three, or four CKX5 or CKX3 genes can be reduced.

Thus, in a first aspect, a Brassica plant is provided comprising at least one, preferably two CKX5 genes, characterised in that it comprises at least one mutant CKX5 allele in its genome. In a further aspect, a Brassica plant is provided comprising at least one CKX5 and at least two CKX3 genes, preferably two CKX5 genes and four CKX3 genes, characterised in that it comprises at least one mutant CKX5 allele and one mutant CKX3 in its genome.

Said Brassica plant comprising at least one CKX5 gene, preferably two CKX5 genes wherein expression of at least one CKX5 gene is reduced, and comprising at least two preferably four CKX3 genes wherein expression of at least on CKX3 gene is reduced can, for example, be a Brassica plant comprising two CKX5 genes, said Brassica plant selected from the group consisting of Brassica napus, Brassica juncea and Brassica carinata. In said Brassica plant, expression of at least one, or at least two, or at least three, or four CKX3 genes can be reduced and/or expression of at least one, or two CKX5 genes can be reduced. The plants according to the invention may, according to this invention, be used for breeding purposes.

In another aspect of the invention, a mutant allele of a Brassica CKX3 or CKX5 gene is provided, wherein the CKX3 gene is selected from the group consisting of:
a nucleotide sequence which comprises at least 90% sequence identity to SEQ ID NO: 7, SEQ ID NO: 10; SEQ ID NO: 13 or SEQ ID NO: 16;
a nucleotide sequence comprising a coding sequence which comprises at least 90% sequence identity to SEQ ID NO: 8, SEQ ID NO: 11; SEQ ID NO: 14 or SEQ ID NO: 17; and
a nucleotide sequence encoding an amino acid sequence which comprises at least 90% sequence identity SEQ ID NO: 9, SEQ ID NO: 12; SEQ ID NO: 15 or SEQ ID NO: 18; and
   wherein the CKX5 gene comprising a nucleic acid sequence selected from the group consisting of:
a nucleotide sequence which comprises at least 90% sequence identity to SEQ ID NO: 19, SEQ ID NO: 22; SEQ ID NO: 23;
a nucleotide sequence comprising a coding sequence which comprises at least 90% sequence identity to SEQ ID NO: 20 or SEQ ID NO: 23; and
a nucleotide sequence encoding an amino acid sequence which comprises at least 90% sequence identity to SEQ ID NO: 21, or SEQ ID NO: 24.

In another embodiment, said mutant allele is selected from the group consisting of:
a mutant CKX5 allele comprising a G to A substitution at a position corresponding to position 465 of SEQ ID NO: 19 or position 465 pf SEQ ID No. 20;
a mutant CKX5 allele comprising a G to A substitution at a position corresponding to position 399 of SEQ ID NO: 19 or position 399 of SEQ ID No. 20;
a mutant CKX5 allele comprising a G to A substitution at a position corresponding to position 465 of SEQ ID NO: 22 or position 399 of SEQ ID No. 23;
a mutant CKX3 allele comprising a C to T substitution at a position corresponding to position 2244 of SEQ ID NO: 7 or position 1093 of SEQ ID No. 8;
a mutant CKX3 allele comprising a C to T substitution at a position corresponding to position 2482 of SEQ ID NO: 10 or position 1168 of SEQ ID No. 11;
a mutant CKX3 allele comprising a G to A substitution at a position corresponding to position 1893 of SEQ ID NO: 13 or position 876 of SEQ ID No. 14;
a mutant CKX3 allele comprising a C to T substitution at a position corresponding to position 2171 of SEQ ID NO: 16 or position 982 of SEQ ID No. 17.

Also provided are methods of generating and combining mutant and wild type CKX alleles in Brassica plants, whereby flower or pod number or TSW is increased in these plants. The use of these plants for transferring mutant CKX alleles to other plants is also an embodiment of the invention, as are the plant products of any of the plants described. In addition kits and methods for marker assisted selection (MAS) for combining or detecting CKX genes and/or alleles are provided. Each of the embodiments of the invention is described in detail herein below.

### Nucleic Acids According to the Invention

Provided are both wild type CKX3 and CKX5 nucleic acid sequences encoding functional CKX3 and CKX5 proteins and mutant CKX3 and CKX5 nucleic acid sequences (comprising one or more mutations, preferably mutations which result in no or a significantly reduced biological activity of the encoded CKX3 or CKX5 protein or in no CKX3 or CKX5 protein being produced) of CKX3 and CKX5 genes from Brassicaceae, particularly from Brassica species, especially from Brassica napus. For example, Brassica species comprising an A and/or a C genome may comprise different alleles of CKX3-A or CKX3-C or CKX5-A or CKX5-C genes, which can be identified and combined in a single plant according to the invention. In addition, mutagenesis or gene targeting methods can be used to generate mutations in wild type CKX3 and CKX5 alleles, thereby generating mutant CKX3 and CKX5 alleles for use according to the invention. Because specific CKX3 and CKX5 alleles are preferably combined in a plant by crossing and selection, in one embodiment the CKX3 and/or CKX5 nucleic acid sequences are provided within a plant (i.e. endogenously), e.g. a Brassica plant, preferably a Brassica plant which can be crossed with Brassica napus or which can be used to make a “synthetic” Brassica napus plant. Hybridization between different Brassica species is described in the art, e.g., as referred to in Snowdon (2007, Chromosome research 15: 85-95). Interspecific hybridization can, for example, be used to transfer genes from, e.g., the C genome in B. napus (AACC) to the C genome in B. carinata (BBCC), or even from, e.g., the C genome in B. napus (AACC) to the B genome in B. juncea (AABB) (by the sporadic event of illegitimate recombination between their C and B genomes). “Resynthesized” or “synthetic” Brassica napus lines can be produced by crossing the original ancestors, B. oleracea (CC) and B. rapa (AA). Interspecific, and also intergeneric, incompatibility barriers can be successfully overcome in crosses between Brassica crop species and their relatives, e.g., by embryo rescue techniques or protoplast fusion (see e.g. Snowdon, above).

However, isolated CKX3 and CKX5nucleic acid sequences (e.g. isolated from the plant by cloning or made synthetically by DNA synthesis), as well as variants thereof and fragments of any of these are also provided herein, as these can be used to determine which sequence is present endogenously in a plant or plant part, whether the sequence encodes a functional, a non-functional or no protein (e.g. by expression in a recombinant host cell as described below) and for selection and transfer of specific alleles from one plant into another, in order to generate a plant having the desired combination of functional and mutant alleles.

Nucleic acid sequences of CKX3 and CKX5 genes have been isolated from Brassica napus (BnCKX3-A1, BnCKX3-A2, BnCKX3-C1, and BnCKX3-C2; BnCKX5-A1 and BnCKX5-C1) as depicted in the sequence listing. The wild type CKX3 and CKX5 sequences are depicted, while the mutant CKX sequences of these sequences, and of sequences essentially similar to these, are described herein below and in the Examples, with reference to the wild type CKX3 and CKX5 sequences. The genomic CKX3 and CKX5 protein-encoding DNA from Brassica napus, Brassica rapa, Brassica oleracea and Brassica nigra contains four introns.

“BnCKX3-A1 nucleic acid sequences” or “BnCKX3-A1 variant nucleic acid sequences” according to the invention are nucleic acid sequences encoding an amino acid sequence having at least 75%, at least 80%, at least 85%, at least 90%, at least 95%, 98%, 99% or 100% sequence identity with SEQ ID NO: 9 or nucleic acid sequences having at least 80%, at least 85%, at least 90%, at least 95%, 96%, 97%, 98%, 99% or 100% sequence identity with SEQ ID NO: 7 or having a cDNA sequence having at least 80%, at least 85%, at least 90%, at least 95%, 96%, 97%, 98%, 99% or 100% sequence identity with SEQ ID NO: 8. These nucleic acid sequences may also be referred to as being “essentially similar” or “essentially identical” to the CKX3 sequences provided in the sequence listing.

“BnCKX3-A2 nucleic acid sequences” or “BnCKX3-A2 variant nucleic acid sequences” according to the invention are nucleic acid sequences encoding an amino acid sequence having at least 75%, at least 80%, at least 85%, at least 90%, at least 95%, 98%, 99% or 100% sequence identity with SEQ ID NO: 12 or nucleic acid sequences having at least 80%, at least 85%, at least 90%, at least 95%, 96%, 97%, 98%, 99% or 100% sequence identity with SEQ ID NO: 10 or having a cDNA sequence having at least 80%, at least 85%, at least 90%, at least 95%, 96%, 97%, 98%, 99% or 100% sequence identity with SEQ ID NO: 11. These nucleic acid sequences may also be referred to as being “essentially similar” or “essentially identical” to the CKX sequences provided in the sequence listing.

“BnCKX3-C1 nucleic acid sequences” or “BnCKX3-C1 variant nucleic acid sequences” according to the invention are nucleic acid sequences encoding an amino acid sequence having at least 75%, at least 80%, at least 85%, at least 90%, at least 95%, 98%, 99% or 100% sequence identity with SEQ ID NO: 15 or nucleic acid sequences having at least 80%, at least 85%, at least 90%, at least 95%, 96%, 97%, 98%, 99% or 100% sequence identity with SEQ ID NO: 13, or having a cDNA sequence having at least 80%, at least 85%, at least 90%, at least 95%, 96%, 97%, 98%, 99% or 100% sequence identity with SEQ ID NO: 14. These nucleic acid sequences may also be referred to as being “essentially similar” or “essentially identical” to the CKX sequences provided in the sequence listing.

“BnCKX3-C2 nucleic acid sequences” or “BnCKX-C2 variant nucleic acid sequences” according to the invention are nucleic acid sequences encoding an amino acid sequence having at least 75%, at least 80%, at least 85%, at least 90%, at least 95%, 98%, 99% or 100% sequence identity with SEQ ID NO: 18 or nucleic acid sequences having at least 80%, at least 85%, at least 90%, at least 95%, 96%, 97%, 98%, 99% or 100% sequence identity with SEQ ID NO: 16 or having a cDNA sequence having at least 80%, at least 85%, at least 90%, at least 95%, 96%, 97%, 98%, 99% or 100% sequence identity with SEQ ID NO: 17. These nucleic acid sequences may also be referred to as being “essentially similar” or “essentially identical” to the CKX sequences provided in the sequence listing.

“BnCKX5-A1 nucleic acid sequences” or “BnCKX5-A1 variant nucleic acid sequences” according to the invention are nucleic acid sequences encoding an amino acid sequence having at least 75%, at least 80%, at least 85%, at least 90%, at least 95%, 98%, 99% or 100% sequence identity with SEQ ID NO: 21 or nucleic acid sequences having at least 80%, at least 85%, at least 90%, at least 95%, 96%, 97%, 98%, 99% or 100% sequence identity with SEQ ID NO: 19 or having a cDNA sequence having at least 80%, at least 85%, at least 90%, at least 95%, 96%, 97%, 98%, 99% or 100% sequence identity with SEQ ID NO: 20. These nucleic acid sequences may also be referred to as being “essentially similar” or “essentially identical” to the CKX3 sequences provided in the sequence listing.

“BnCKX5-C1 nucleic acid sequences” or “BnCKX5-C1 variant nucleic acid sequences” according to the invention are nucleic acid sequences encoding an amino acid sequence having at least 75%, at least 80%, at least 85%, at least 90%, at least 95%, 98%, 99% or 100% sequence identity with SEQ ID NO: 24 or nucleic acid sequences having at least 80%, at least 85%, at least 90%, at least 95%, 96%, 97%, 98%, 99% or 100% sequence identity with SEQ ID NO: 22, or having a cDNA sequence having at least 80%, at least 85%, at least 90%, at least 95%, 96%, 97%, 98%, 99% or 100% sequence identity with SEQ ID NO: 23. These nucleic acid sequences may also be referred to as being “essentially similar” or “essentially identical” to the CKX sequences provided in the sequence listing.

Thus the invention provides both nucleic acid sequences encoding wild type, functional CKX3 and CKX5 proteins, including variants and fragments thereof (as defined further below), as well as mutant nucleic acid sequences of any of these, whereby the mutation in the nucleic acid sequence preferably results in one or more amino acids being inserted, deleted or substituted in comparison to the wild type CKX3 or CKX5 protein. Preferably the mutation(s) in the nucleic acid sequence result in one or more amino acid changes (i.e. in relation to the wild type amino acid sequence one or more amino acids are inserted, deleted and/or substituted) whereby the biological activity of the CKX3 or CKX5 protein is significantly reduced or completely abolished. A significant reduction in or complete abolishment of the biological activity of the CKX3 or CKX5 protein refers herein to a reduction in or abolishment of the substrate binding activity and/or the catalytic capacity of the CKX3 or CKX5 protein, such that flower number, pod number and/or TSW of a plant expressing the mutant CKX3 or CKX5 protein is increased as compared to a plant expressing the corresponding wild type CKX3 or CKX5 protein.

To determine the functionality of a specific CKX allele/protein in plants, particularly in Brassica plants, the number of flowers on the plants can be determined by counting as described herein in the Examples below, and/or by microscopic tests to examine, e.g., whether and how meristems, particularly flower meristems are affected by mutations in CKX5 or CKX5 and CKX3. The functionality of a specific CKX3 or CKX5 allele/protein can alternatively be evaluated by recombinant DNA techniques as known in the art, e.g., by expressing CKX3 or CKX5 in a host cell (e.g. a bacterium, such as E. coli) and evaluating e.g. substrate binding activity or in vitro catalysis of the oxidation of cytokinin, such as isopentenyl adenine.

Both endogenous and isolated nucleic acid sequences are provided herein. Also provided are fragments of the CKX3 or CKX5 sequences and CKX3 or CKX5 variant nucleic acid sequences defined above, for use as primers or probes and as components of kits according to another aspect of the invention (see further below). A “fragment” of a CKX3 or CKX5 or CKX nucleic acid sequence or variant thereof (as defined) may be of various lengths, such as at least 10, 12, 15, 18, 20, 50, 100, 200, 500, 800, 1000, or 1500 contiguous nucleotides of the respective CKX or CKX sequence (or of the variant sequence).

### Nucleic Acid Sequences Encoding Functional CKX3 or CKX5 Proteins

The nucleic acid sequences depicted in the sequence listing encode wild type, functional CKX3 or CKX5 proteins from Brassica napus. Thus, these sequences are endogenous to the Brassica plants from which they were isolated. Other Brassica crop species, varieties, breeding lines or wild accessions may be screened for other CKX3 or CKX5 alleles, encoding the same CKX3 or CKX5 proteins or variants thereof. For example, nucleic acid hybridization techniques (e.g. Southern blot analysis, using for example stringent hybridization conditions) or PCR-based techniques may be used to identify CKX3 or CKX5 alleles endogenous to other Brassica plants, such as various Brassica napus varieties, lines or accessions, but also Brassica juncea (especially CKX3 or CKX5 or alleles on the A-genome), Brassica carinata (especially CKX3 or CKX5 alleles on the C-genome) and Brassica rapa (especially CKX3 or CKX5 alleles on the A-genome) and Brassica oleracea (especially CKX3 or CKX5 alleles on the C-genome) plants, organs and tissues can be screened for other wild type CKX3 or CKX5 alleles. To screen such plants, plant organs or tissues for the presence of CKX3 or CKX5 alleles, the CKX3 or CKX5 nucleic acid sequences provided in the sequence listing, or variants or fragments of any of these, may be used. For example whole sequences or fragments may be used as probes or primers. For example specific or degenerate primers may be used to amplify nucleic acid sequences encoding CKX3 or CKX5 proteins from the genomic DNA of the plant, plant organ or tissue. These CKX3 or CKX5 nucleic acid sequences may be isolated and sequenced using standard molecular biology techniques. Bioinformatics analysis may then be used to characterize the allele(s), for example in order to determine which CKX3 or CKX5 allele the sequence corresponds to and which CKX5 or CKX3 protein or protein variant is encoded by the sequence.

Whether a nucleic acid sequence encodes a functional CKX3 or CKX5 protein can be analyzed by recombinant DNA techniques as known in the art, e.g., by a genetic complementation test using, e.g., an Arabidopsis plant, which is homozygous for a full knock-out ckx3 or ckx5 mutant allele (or both) or a Brassica napus plant, which is homozygous for a full knock-out ck3 or ckx5 mutant allele of both, or all of the CKX3-A1, CKX3-A2, CKX3-C1 and/or the CKX3-C2 gene and/or the CKX5-A1 and CKX5-C1.

In addition, it is understood that CKX3 or CKX5 nucleic acid sequences and variants thereof (or fragments of any of these) may be identified in silico, by screening nucleic acid databases for essentially similar sequences. Likewise, a nucleic acid sequence may be synthesized chemically. Fragments of nucleic acid molecules according to the invention are also provided, which are described further below.

### Nucleic Acid Sequences Encoding Mutant CKX3 or CKX5 Proteins

Nucleic acid sequences comprising one or more nucleotide deletions, insertions or substitutions relative to the wild type nucleic acid sequences are another embodiment of the invention, as are fragments of such mutant nucleic acid molecules. Such mutant nucleic acid sequences (referred to as ckx3 or ckx5 sequences) can be generated and/or identified using various known methods, as described further below. Again, such nucleic acid molecules are provided both in endogenous form and in isolated form. In one embodiment, the mutation(s) result in one or more changes (deletions, insertions and/or substitutions) in the amino acid sequence of the encoded CKX3 or CKX5 protein (i.e. it is not a “silent mutation”). In another embodiment, the mutation(s) in the nucleic acid sequence result in a significantly reduced or completely abolished biological activity of the encoded CKX3 or CKX5 protein relative to the wild type protein.

The nucleic acid molecules may, thus, comprise one or more mutations, such as:
(a) a “missense mutation”, which is a change in the nucleic acid sequence that results in the substitution of an amino acid for another amino acid;
(b) a “nonsense mutation” or “STOP codon mutation”, which is a change in the nucleic acid sequence that results in the introduction of a premature STOP codon and thus the termination of translation (resulting in a truncated protein); plant genes contain the translation stop codons “TGA” (UGA in RNA), “TAA” (UAA in RNA) and “TAG” (UAG in RNA); thus any nucleotide substitution, insertion, deletion which results in one of these codons to be in the mature mRNA being translated (in the reading frame) will terminate translation;
(c) an “insertion mutation” of one or more amino acids, due to one or more codons having been added in the coding sequence of the nucleic acid;
(d) a “deletion mutation” of one or more amino acids, due to one or more codons having been deleted in the coding sequence of the nucleic acid;
(e) a “frameshift mutation”, resulting in the nucleic acid sequence being translated in a different frame downstream of the mutation. A frameshift mutation can have various causes, such as the insertion, deletion or duplication of one or more nucleotides, which number is not dividable by 3.

As already mentioned, it is desired that the mutation(s) in the nucleic acid sequence preferably result in a mutant protein comprising significantly reduced or no biological activity in vivo or in the production of no protein, Basically, any mutation which results in a protein comprising at least one amino acid insertion, deletion and/or substitution relative to the wild type protein can lead to significantly reduced or no biological activity. It is, however, understood that mutations in certain parts of the protein are more likely to result in a reduced function of the mutant CKX3 or CKX5 protein, such as mutations leading to truncated proteins, whereby significant portions of the functional domains, such as the FAD-binding motif, the cytokinin binding motif, the GIWeVPHPWLNL motif, and/or the PGQxIF motif, are lacking.

Amino acid positions of the conserved motifs and catalytic residues in the Arabidopsis and Brassica CKX3 and CKX5 protein sequences are indicated in Tables 1 and 2.

TABLE 1
conserved regions in CKX3 proteins from A. thaliana and B. napus

| | | | Bn-CKX3- | Bn-CKX3- | Bn-CKX3- | Bn-CKX3- | | | | |
|---|---|---|---|---|---|---|---|---|---|---|
| | | AtCKX3 | A1 | A2 | C1 | C2 | YIIN501 | YIIN512 | YIIN521 | YIIN531 |
| SEQ ID No.: | | 3 | 9 | 12 | 15 | 18 | 25 | 26 | 27 | 28 |
| signal peptide | \|_(1)_\| | 1 to 31 | 1 to 32 | 1 to 30 | 1 to 32 | 1 to 31 | 1 to 32 | 1 to 30 | 1 to 32 | 1 to 31 |
| FAD-binding PCHM- | \|_(2)_\| | 66 to 243 | 67 to 244 | 65 to 242 | 67 to 244 | 66 to 243 | 67 to 244 | 65 to 242 | 67 to 244 | 66 to 243 |
| type | | | | | | | | | | |
| FAD-binding | ▴ | 100 to 104 | 101 to 105 | 99 to 103 | 101 to 105 | 100 to 104 | 101 to 105 | 99 to 103 | 101 to 105 | 100 to 104 |
| FAD-binding | ▴ | 105 to 106 | 106 to 107 | 104 to 105 | 106 to 107 | 105 to 106 | 106 to 107 | 104 to 105 | 106 to 107 | 105 to 106 |
| Pros-8alpha-FAD | ▴ | 105 | 106 | 104 | 106 | 105 | 106 | 104 | 106 | 105 |
| Histidine | | | | | | | | | | |
| FAD-binding via | ▴ | 110 | 111 | 109 | 111 | 110 | 111 | 109 | 111 | 110 |
| carbonyl oxygen | | | | | | | | | | |
| Glycosylation | ⋄ | 153 | 154 | 152 | 154 | 153 | 154 | 152 | 154 | 153 |
| FAD-binding via | ▴ | 167 | 168 | 166 | 168 | 167 | 168 | 166 | 168 | 167 |
| amide nitrogen | | | | | | | | | | |
| FAD-binding | ▴ | 172 | 173 | 171 | 173 | 172 | 173 | 171 | 173 | 172 |
| FAD-binding | ▴ | 178 to 182 | 179 to 183 | 177 to 181 | 179 to 183 | 178 to 182 | 179 to 183 | 177 to 181 | 179 to 183 | 178 to 182 |
| FAD-binding via | ▴ | 233 | 234 | 233 | 234 | 233 | 234 | 233 | 234 | 233 |
| amide nitrogen | | | | | | | | | | |
| and carbonyl | | | | | | | | | | |
| oxygen | | | | | | | | | | |
| Cytoninin binding | \|_(3)_\| | 244 to 517 | 245 to 518 | 243 to 516 | 245 to 518 | 244 to 517 | not | not | not | not |
| | | | | | | | present | present | present | present |
| Glycosylation | ⋄ | 408 | 409 | 407 | 409 | 408 | not | not | not | not |
| | | | | | | | present | present | present | present |
| FAD-binding | ▴ | 476 | 477 | 475 | 477 | 476 | not | not | not | not |
| | | | | | | | present | present | present | present |
| GIWeVPHPWLNL | ▪ | 374 to 385 | 375 to 386 | 373 to 384 | 375 to 386 | 374 to 385 | not | 373 to 384 | not | not |
| motif | | | | | | | present | | present | present |
| PGQxIF motif | ▪ | 512 to 517 | 513 to 518 | 511 to 516 | 513 to 518 | 512 to 517 | not | not | not | not |
| | | | | | | | present | present | present | present |

TABLE 2
conserved regions in CKX5 proteins from A. thaliana and B. napus

| | | AtCKX5 | Bn-CKX5-A1 | Bn-CKX5-C1 | YIIN801 | YIIN805 | YIIN811 |
|---|---|---|---|---|---|---|---|
| SEQ ID No.: | | 6 | 21 | 24 | 29 | 30 | 31 |
| signal peptide | \|_(1)_\| | 1 to 24 | 1 to 18 | 1 to 18 | 1 to 18 | 1 to 18 | 1 to 18 |
| FAD-binding PCHM-type | \|_(2)_\| | 63 to 241 | 55 to 233 | 55 to 233 | not present | not present | not present |
| FAD-binding | ▴ | 97 to 101 | 89 to 93 | 89 to 93 | 89 to 93 | 89 to 93 | 89 to 93 |
| FAD-binding | ▴ | 102-103 | 94 to 95 | 94 to 95 | 94 to 95 | 94 to 95 | 94 to 95 |
| Pros-8alpha-FAD Histidine | ▴ | 102 | 94 | 94 | 94 | 94 | 94 |
| FAD-binding via carbonyl oxygen | ▴ | 107 | 99 | 99 | 99 | 99 | 99 |
| FAD-binding via amide nitrogen | ▴ | 165 | 157 | 157 | not present | not present | not present |
| FAD-binding | ▴ | 170 | 162 | 162 | not present | not present | not present |
| FAD-binding | ▴ | 176 to 180 | 168 to 172 | 168 to 172 | not present | not present | not present |
| FAD-binding via amide nitrogen | ▴ | 231 | 223 | 223 | not present | not present | not present |
| and carbonyl oxygen | | | | | | | |
| Cytoninin binding | \|_(3)_\| | 242 to 520 | 234 to 512 | 234 to 512 | not present | not present | notpresent |
| Glycosylation | ⋄ | 310 | 302 | 302 | not present | not present | not present |
| Glycosylation | ⋄ | 406 | 398 | 398 | not present | not present | not present |
| FAD-binding | ▴ | 479 | 471 | 471 | not present | not present | not present |
| GIWeVPHPWLNL motif | ▪ | 374 to 385 | 368 to 377 | 368 to 377 | not present | not present | not present |
| PGQxIF motif | ▪ | 515 to 520 | 507 to 512 | 507 to 512 | not present | not present | not present |

Optimal alignment of the Arabidopsis CKX3 and CKX5 nucleic acid (SEQ ID NOs: 1, 2, 4 and 5) and amino acid (SEQ ID NO: 3 and 6) sequences with CKX3 and CKX5 nucleic acid sequences, in particular the Brassica CKX3 and CKX5 nucleic acid and amino acid sequences of the present invention, allows to determine the positions of the corresponding conserved domains and amino acids in these Brassica sequences (see Tables 1 and 2 for the Brassica CKX3 and CKX5 sequences).

Thus in one embodiment, nucleic acid sequences comprising one or more of any of the types of mutations described above are provided. In another embodiment, ckx3/ckx5-sequences comprising one or more stop codon (nonsense) mutations, one or more missense mutations and/or one or more frameshift mutations are provided. Any of the above mutant nucleic acid sequences are provided per se (in isolated form), as are plants and plant parts comprising such sequences endogenously. In the tables herein below the most preferred ckx3/ckx5 alleles are described and seed deposits of Brassica napus seeds comprising one or more ckx3/ckx5 alleles have been deposited as indicated.

A nonsense mutation in a CKX3 or CKX5 allele, as used herein, is a mutation in a CKX3 or CKX5 allele whereby one or more translation stop codons are introduced into the coding DNA and the corresponding mRNA sequence of the corresponding wild type CKX3 or CKX5 allele. Translation stop codons are TGA (UGA in the mRNA), TAA (UAA) and TAG (UAG). Thus, any mutation (deletion, insertion or substitution) that leads to the generation of an in-frame stop codon in the coding sequence will result in termination of translation and truncation of the amino acid chain. In one embodiment, a mutant CKX3 or CKX5 allele comprising a nonsense mutation is a CKX3 or CKX5 allele wherein an in-frame stop codon is introduced in the CKX3 or CKX5 codon sequence by a single nucleotide substitution, such as the mutation of CAG to TAG, TGG to TAG, TGG to TGA, or CAA to TAA. In another embodiment, a mutant CKX3 or CKX5 allele comprising a nonsense mutation is a CKX3 or CKX5 allele wherein an in-frame stop codon is introduced in the CKX3 or CKX5 codon sequence by double nucleotide substitutions, such as the mutation of CAG to TAA, TGG to TAA, or CGG to TAG or TGA. In yet another embodiment, a mutant CKX3 or CKX5 allele comprising a nonsense mutation is a CKX3 or CKX5 allele wherein an in-frame stop codon is introduced in the CKX3 or CKX5 codon sequence by triple nucleotide substitutions, such as the mutation of CGG to TAA. The truncated protein lacks the amino acids encoded by the coding DNA downstream of the mutation (i.e. the C-terminal part of the CKX3 or CKX5 protein) and maintains the amino acids encoded by the coding DNA upstream of the mutation (i.e. the N-terminal part of the CKX3 or CKX5 protein). In one embodiment, a mutant CKX3 or CKX5 allele comprising a nonsense mutation is a CKX3 or CKX5e wherein the nonsense mutation is present anywhere in front of the PGQXIF-motif at positions corresponding to 512-517 of SEQ ID NO: 3, so that at least the conserved domain PGQXIF is lacking. The more truncated the mutant CKX3 or CKX5 protein is in comparison to the wild type CKX3 or CKX5 protein, the more the truncation may result in a significantly reduced or no activity of the CKX3 or CKKX5 protein. Thus in another embodiment, a mutant CKX3 or CKX5 allele comprising a nonsense mutation which results in a truncated protein of less than about 517 or 518, or 516 (lacking a complete cytokinin binding site), less than about 244 or 245, or 243 (lacking the complete cytokinin binding site), less than about 233, or 234 amino acids (lacking the FAD-binding amino acid at position 233) or even less amino acids in length. See Tables 1 and 2 for indication conserved regions and domains which are not any longer present in the particular YIIN alleles.

It will be clear as described herein in the examples that the CKX alleles that are truncated at a position corresponding to position 364 of SEQ ID NO: 3 (YINN501) or corresponding to position 389 of SEQ ID NO. 3, lacking a complete cytokinin binding motif, the GIWeVPHPWLNL motif, and the PGQxIF motif as well as the FAD binding site at a position corresponding to position 476 of SEQ ID No. 3 or position 479 of SEQ ID No. 6, which are the longest truncated CKX proteins of the Examples, contribute to an increase of flower number and TSW. Therefore, in a particular embodiment, the CKX3 or CKX5 allele according to the invention encodes a truncated protein lacking the GIWeVPHPWLNL motif, and the PGQxIF motif as well as the FAD binding site at a position corresponding to position 476.

Obviously, mutations are not limited to the ones indicated above and it is understood that analogous STOP mutations may be present in ckx3/ckx5 alleles other than those depicted in the sequence listing and referred to in the tables above.

A missense mutation in a CKX3 or CKX5 allele, as used herein, is any mutation (deletion, insertion or substitution) in a CKX3 or CKX5 allele whereby one or more codons are changed into the coding DNA and the corresponding mRNA sequence of the corresponding wild type CKX3 or CKX5 allele, resulting in the substitution of one or more amino acids in the wild type CKX3 or CKX5 protein for one or more other amino acids in the mutant CKX3 or CKX5 protein. In one embodiment, a mutant CKX3 or CKX5 allele comprising a missense mutation is a CKX3 or CKX5 allele wherein one or more of the conserved amino acids indicated above or in Tables 1 or 2 is/are substituted. Missense mutations which result in the substitution of, e.g., the amino acid at a position corresponding to position 100 to 104, 105 to 106, 110, 153, 167, 172, 178 to 182, 233, 476, 374 to 385 or 512 to 517 of SEQ ID NO: 3 are more likely to result in a significantly reduced or no activity, of the CKX3 protein. Similarly missense mutations which result in the substitution of, e.g., the amino acid at a position corresponding to position 97 to 101, 102 to 103, 107, 165, 170, 176 to 180, 231, 479, 374 to 385 or 515 to 520 of SEQ ID NO: 6 are more likely to result in a significantly reduced or no activity of the CKX5 protein.

A frameshift mutation in a CKX3 or CKX5 allele, as used herein, is a mutation (deletion, insertion, duplication, and the like) in a CKX3 or CKX5 allele that results in the nucleic acid sequence being translated in a different frame downstream of the mutation. In one embodiment, a mutant CKX3 or CKX5 allele comprising a frameshift mutation is a CKX3 or CKX5 allele comprising a frameshift mutation upstream of the codon encoding the first amino acid of the PGQxIF motif corresponding to position 512 of SEQ ID NO: 3 or 515 of SEQ ID NO. 6, or comprising a frameshift mutation upstream of the codon encoding the first amino acid of the GIWeVPHPWLNL motif corresponding to position 374 of SEQ ID NO: 3 or 374 of SEQ ID NO. 6, or comprising a frameshift mutation upstream of the codon encoding the first amino acid of the cytokinin binding motif corresponding to position 244 of SEQ ID NO: 3 or 242 of SEQ ID NO. 6, or comprising a frameshift mutation upstream of the codon encoding the first amino acid of the FAD motif corresponding to position 66 of SEQ ID NO: 3 or position 63 of SEQ ID NO. 6, or comprising a frameshift mutation upstream of the codon encoding the FAD binding amino acids at the amino acid at a position corresponding to position 100 to 104, 105 to 106, 110, 153, 167, 172, 178 to 182, 233, 476, 374 to 385 or 512 to 517 of SEQ ID NO: 3 or corresponding to position 97 to 101, 102 to 103, 107, 165, 170, 176 to 180, 231, 479, 374 to 385 or 515 to 520 of SEQ ID NO: 6.

A mutant CKX3 or CKX5 allele may also be a CKX3 or CKX5 allele which produces no CKX3 or CKX5 protein. Examples of mutant alleles that do not produce a protein are alleles having mutations leading to no production or degradation of the mRNA, such as mutations in promoter regions abolishing mRNA production, stop codon mutations leading to degradation of the mRNA (nonsense-mediated decay; see, for example, Baker and Parker, 2004, Curr Opin Cell Biol 16:293), splice site mutations leading to RNA degradation (see, for example, Isken and Maquat, 2007, Genes Dev 21:1833), or mutations in the protein coding sequence comprising mutation or deletion of the ATG start codon, such that no protein is produced, or gross deletions in the gene leading to absence of (part of) the protein coding sequence.

The mutant CKX3 or CKX5 alleles according to the invention can thus comprise nucleotide sequences which comprise at least 90% but less than 100% sequence identity to SEQ ID NO: 7, SEQ ID NO: 10, SEQ ID NO: 13, SEQ ID NO: 16, SEQ ID NO: 19 or SEQ ID NO: 22; or can comprise nucleotide sequences comprising a coding sequence which comprises at least 90% but less than 100% sequence identity to SEQ ID NO: 8, SEQ ID NO: 11, SEQ ID NO: 14, SEQ ID NO: SEQ ID NO: 17, SEQ ID NO: 20 or SEQ ID NO: 23; or can comprise nucleotide sequences encoding an amino acid sequence which comprises at least 90% but less than 100% sequence identity to SEQ ID NO: 9, SEQ ID NO: 12, SEQ ID NO: 15, SEQ ID NO: 18, SEQ ID NO: 21 or SEQ ID NO: 24. Said at least 90% can be at least 90%, or at least 93%, or at least 95%, or at least 96%, or at least 97%, or at least 98%, or 99%. However, the mutant CKX3 or CKX5 alleles according to the invention cannot comprise nucleotide sequences comprising 100% sequence identity to the above sequences. Furthermore, the mutant CKX3 or CKX5 alleles according to the invention can comprise sequence identity which is lower than 90% to the above-mentioned sequences, such as, for example, when part or all of the wild type CKX3 or CKX5 gene is deleted. In such a case, a mutant CKX3 or CKX5 allele may also refer to a genetic locus corresponding to the genetic locus of a wild type CKX3 or CKX5 allele, wherein a CKX3 or CKX5 allele is present having less than 100% sequence identity to the wild type allele, or wherein a part of, or the complete CKX3 or CKX5 gene, is deleted.

### Amino Acid Sequences According to the Invention

Provided are both wild type (functional) CKX3 or CKX5 amino acid sequences and mutant CKX3 or CKX5 amino acid sequences (comprising one or more mutations, preferably mutations which result in a significantly reduced or no biological activity of the CKX3 or CKX5 protein) from Brassicaceae, particularly from Brassica species, especially from Brassica napus, Brassica rapa, Brassica oleracea and Brassica nigra, but also from other Brassica crop species. For example, Brassica species comprising an A and/or a C genome may encode different CKX3-A or CKX5-A or CKX3-C or CKX5-C amino acids. In addition, mutagenesis or gene targeting methods can be used to generate mutations in wild type CKX3 or CKX5 alleles, thereby generating mutant alleles which can encode further mutant CKX3 or CKX5 proteins. In one embodiment the wild type and/or mutant CKX3 or CKX5 amino acid sequences are provided within a Brassica plant (i.e. endogenously). However, isolated CKX3 or CKX5 amino acid sequences (e.g. isolated from the plant or made synthetically), as well as variants thereof and fragments of any of these are also provided herein.

A significantly reduced or no biological activity of the CKX5 or CKX3 protein can be a reduction of at least 10%, or of at least 20%, or of at least 40%, or of at least 60%, or of at least 80%, or of at least 90%, or of at least 95%, or of at least 98%, or a reduction of 100% in which no protein activity can be detected, as compared to a functional CKX5 or CKX3 protein, such as a functional CKX5 or CKX3 protein encoded by a wild type CKX3 or CKX5 allele. Cytokinin oxidase activity can be determined, for example, as described by Liberos-Minotta and Tipton (1995) Analytical Biochemistry 231, 339-341 or Frebort et al.(2002) Analytical Biochemistry 306, 1-7 (both incorporated herein by reference).

Amino acid sequences of CKX3 and CKX5 proteins have been isolated from Brassica napus, as depicted in the sequence listing. The wild type CKX3 and CKX5 sequences are depicted, while the mutant CKX3 and CKX5 sequences of these sequences, and of sequences essentially similar to these, are described herein below, with reference to the wild type CKX3 and CKX5 sequences.

As described above, the CKX3 or CKX5 proteins of Brassica described herein are about 520 amino acids in length and comprise a number of structural and functional domains.

“BnCKX3-A1 amino acid sequences” or “BnCKX3-A1 variant amino acid sequences” according to the invention are amino acid sequences having at least 75%, at least 80%, at least 85%, at least 90%, at least 95%, 98%, 99% or 100% sequence identity with SEQ ID NO 9. These amino acid sequences may also be referred to as being “essentially similar” or “essentially identical” to the CKX3 sequences provided in the sequence listing.

“BnCKX3-A2 amino acid sequences” or “BnCKX3-A2 variant amino acid sequences” according to the invention are amino acid sequences having at least 75%, at least 80%, at least 85%, at least 90%, at least 95%, 98%, 99% or 100% sequence identity with SEQ ID NO 12. These amino acid sequences may also be referred to as being “essentially similar” or “essentially identical” to the CKX3 sequences provided in the sequence listing.

“BnCKX3-C1 amino acid sequences” or “BnCKX3-C1 variant amino acid sequences” according to the invention are amino acid sequences having at least 75%, at least 80%, at least 85%, at least 90%, at least 95%, 96%, 97%, 98%, 99% or 100% sequence identity with SEQ ID NO 15. These amino acid sequences may also be referred to as being “essentially similar” or “essentially identical” the CKX3 sequences provided in the sequence listing.

“BnCKX3-C2 amino acid sequences” or “BnCKX3-C2 variant amino acid sequences” according to the invention are amino acid sequences having at least 75%, at least 80%, at least 85%, at least 90%, at least 95%, 96%, 97%, 98%, 99% or 100% sequence identity with any one of SEQ ID NOs 18. These amino acid sequences may also be referred to as being “essentially similar” or “essentially identical” the CKX3 sequences provided in the sequence listing.

“BnCKX5-A1 amino acid sequences” or “BnCKX5-A1 variant amino acid sequences” according to the invention are amino acid sequences having at least 75%, at least 80%, at least 85%, at least 90%, at least 95%, 98%, 99% or 100% sequence identity with SEQ ID NO 21. These amino acid sequences may also be referred to as being “essentially similar” or “essentially identical” to the CKX5 sequences provided in the sequence listing.

“BnCKX5-C1 amino acid sequences” or “BnCKX5-C1 variant amino acid sequences” according to the invention are amino acid sequences having at least 75%, at least 80%, at least 85%, at least 90%, at least 95%, 96%, 97%, 98%, 99% or 100% sequence identity with SEQ ID NO 24. These amino acid sequences may also be referred to as being “essentially similar” or “essentially identical” the CKX5 sequences provided in the sequence listing.

Thus, the invention provides both amino acid sequences of wild type, functional CKX3 or CKX5 proteins, including variants and fragments thereof (as defined further below), as well as mutant amino acid sequences of any of these, whereby the mutation in the amino acid sequence preferably results in a significant reduction in or a complete abolishment of the biological activity of the CKX3 or CKX5 protein as compared to the biological activity of the corresponding wild type CKX3 or CKX5 protein. A significant reduction in or complete abolishment of the biological activity of the CKX3 or CKX5 protein refers herein to a reduction in or abolishment of the substrate binding activity or the catalytic activity, such that the flower number, pod number and/or TSW of a plant expressing the mutant CKX3 or CKX5 protein is increased as compared to a plant expressing the corresponding wild type CKX3 or CKX5 protein compared to flower number, pod number and/or TSW of a corresponding wild type plant.

Both endogenous and isolated amino acid sequences are provided herein. Also provided are fragments of the CKX3 or CKX5 amino acid sequences and CKX3 or CKX5 variant amino acid sequences defined above. A “fragment” of a CKX3 or CKX5 amino acid sequence or variant thereof (as defined) may be of various lengths, such as at least 10, 12, 15, 18, 20, 50, 100, 150, 175, 200, 150, 300, 350 or 400 contiguous amino acids of the CKX3 or CKX5 sequence (or of the variant sequence). Examples of such fragments for CKX3 proteins are those consisting of the amino acid sequences of any one of SEQ ID No. 25, SEQ ID No. 26, SEQ ID No. 27 or SEQ ID No. 28 Examples of such fragments for CKX5 proteins are those consisting of the amino acid sequences of any one of SEQ ID No. 29, SEQ ID No. 30, or SEQ ID No. 31.

### Amino Acid Sequences of Functional CKX3 or CKX5 Proteins

The amino acid sequences depicted in the sequence listing are wild type, functional CKX3 or CKX5 proteins from Brassica napus. Thus, these sequences are endogenous to the Brassica plants from which they were isolated. Other Brassica crop species, varieties, breeding lines or wild accessions may be screened for other functional CKX3 or CKX5 proteins with the same amino acid sequences or variants thereof, as described above.

In addition, it is understood that CKX3 or CKX5 amino acid sequences and variants thereof (or fragments of any of these) may be identified in silico, by screening amino acid databases for essentially similar sequences. Fragments of amino acid molecules according to the invention are also provided.

### Amino Acid Sequences of Mutant CKX3 or CKX5 Proteins

Amino acid sequences comprising one or more amino acid deletions, insertions or substitutions relative to the wild type amino acid sequences are another embodiment of the invention, as are fragments of such mutant amino acid molecules. Such mutant amino acid sequences can be generated and/or identified using various known methods, as described above. Again, such amino acid molecules are provided both in endogenous form and in isolated form.

In one embodiment, the mutation(s) in the amino acid sequence result in a significantly reduced or completely abolished biological activity of the CKX3 or CKX5 protein relative to the wild type protein. As described above, basically, any mutation which results in a protein comprising at least one amino acid insertion, deletion and/or substitution relative to the wild type protein can lead to significantly reduced or no biological activity. It is, however, understood that mutations in certain parts of the protein are more likely to result in a reduced function of the mutant CKX3 or CKX5 protein, such as mutations leading to truncated proteins, whereby significant portions of the conserved domains, as described in Tables 1 or 2 are lacking or being substituted.

Thus in one embodiment, mutant CKX3 or CKX5 proteins are provided comprising one or more deletion or insertion mutations, whereby the deletion(s) or insertion(s) result(s) in a mutant protein which has significantly reduced or no activity in vivo. Such mutant CKX3 or CKX5 proteins are CKX3 or CKX5 proteins wherein at least 1, at least 2, 3, 4, 5, 10, 20, 30, 50, 100, 100, 150, 175, 180, 200, 250, 300, 350, 400 or more amino acids are deleted or inserted as compared to the wild type CKX3 or CKX5 protein, whereby the deletion(s) or insertion(s) result(s) in a mutant protein which has significantly reduced or no activity in vivo.

In another embodiment, mutant CKX3 or CKX5 proteins are provided which are truncated whereby the truncation results in a mutant protein that has significantly reduced or no activity in vivo. Such truncated CKX3 or CKX5 proteins are CKX3 or CKX5 proteins which lack functional domains in the C-terminal part of the corresponding wild type CKX3 or CKX5 protein and which maintain the N-terminal part of the corresponding wild type CKX3 or CKX5 protein. Thus in one embodiment, a truncated CKX3 or CKX5 protein comprising the N-terminal part of the corresponding wild type CKX3 or CKX5 protein up to but not including the first amino acid of the PGQxIF motif (at a position corresponding to position 512 of SEQ ID NO: 3) is provided. The more truncated the mutant protein is in comparison to the wild type protein, the more the truncation may result in a significantly reduced or no activity of the CKX3 or CKX5 protein. Thus in another embodiment, a trunctated CKX3 or CKX5 protein comprising the N-terminal part of the corresponding wild type CKX3 or CKX5 protein lacking part or all of the FAD binding motif, and/or lacking part or all of the cytokining binding motif (as described above), are provided.

In yet another embodiment, mutant CKX3 or CKX5 proteins are provided comprising one or more substitution mutations, whereby the substitution(s) result(s) in a mutant protein that has significantly reduced or no activity in vivo. Such mutant CKX3 or CKX5 proteins are CKX3 or CKX5 proteins whereby conserved amino acid residues which have a specific function, substrate binding or a catalytic function, as described above, are substituted. Thus in one embodiment, mutant CKX3 or CKX5 proteins comprising a substitution of a conserved amino acid residue which has a biological function, such as the conserved amino acids of the cytokinin binding motif, the FAD-binding motif, the GIWeVPHPWLNL motif, or the PGQxIF motif are provided.

### Methods According to the Invention

Mutant ckx3 or ckx5 alleles may be generated (for example induced by mutagenesis or gene targeting) and/or identified using a range of methods, which are conventional in the art, for example using PCR based methods to amplify part or all of the ckx3 or ckx5 genomic or cDNA.

Following mutagenesis, plants are grown from the treated seeds, or regenerated from the treated cells using known techniques. For instance, mutagenized seeds may be planted in accordance with conventional growing procedures and following self-pollination seed is formed on the plants. Alternatively, doubled haploid plantlets may be extracted from treated microspore or pollen cells to immediately form homozygous plants, for example as described by Coventry et al. (1988, Manual for Microspore Culture Technique for Brassica napus. Dep. Crop Sci. Techn. Bull. OAC Publication 0489. Univ. of Guelph, Guelph, Ontario, Canada). Additional seed which is formed as a result of such self-pollination in the present or a subsequent generation may be harvested and screened for the presence of mutant CKX3 or CKX5 alleles, using techniques which are conventional in the art, for example polymerase chain reaction (PCR) based techniques (amplification of the ckx3/ckx5 alleles) or hybridization based techniques, e.g. Southern blot analysis, BAC library screening, and the like, and/or direct sequencing of ckx3/ckx5 alleles. To screen for the presence of point mutations (so called Single Nucleotide Polymorphisms or SNPs) in mutant CKX3 or CKX5 alleles, SNP detection methods conventional in the art can be used, for example oligoligation-based techniques, single base extension-based techniques or techniques based on differences in restriction sites, such as TILLING.

As described above, mutagenization (spontaneous as well as induced) of a specific wild type CKX3 or CKX5 allele results in the presence of one or more deleted, inserted, or substituted nucleotides (hereinafter called “mutation region”) in the resulting mutant CKX3 or CKX5 allele. The mutant CKX3 or CKX5 allele can thus be characterized by the location and the configuration of the one or more deleted, inserted, or substituted nucleotides in the wild type CKX3 or CKX5 allele. The site in the wild type CKX3 or CKX5 allele where the one or more nucleotides have been inserted, deleted, or substituted, respectively, is herein also referred to as the “mutation region or sequence”. A “5′ or 3′ flanking region or sequence” as used herein refers to a DNA region or sequence in the mutant (or the corresponding wild type) CKX3 or CKX5 allele of at least 20 bp, preferably at least 50 bp, at least 750 bp, at least 1500 bp, and up to 5000 bp of DNA different from the DNA containing the one or more deleted, inserted, or substituted nucleotides, preferably DNA from the mutant (or the corresponding wild type) CKX3 or CKX5 allele which is located either immediately upstream of and contiguous with (5′ flanking region or sequence“) or immediately downstream of and contiguous with (3′ flanking region or sequence”) the mutation region in the mutant CKX3 or CKX5 allele (or in the corresponding wild type CKX3 or CKX5 allele). A “joining region” as used herein refers to a DNA region in the mutant (or the corresponding wild type) CKX3 or CKX5 allele where the mutation region and the 5′ or 3′ flanking region are linked to each other. A “sequence spanning the joining region between the mutation region and the 5′ or 3′ flanking region thus comprises a mutation sequence as well as the flanking sequence contiguous therewith.

Variant CKX5 or CKX3 alleles may also be identified by identifying QTLs for number of flower, number of pods or seeds per pod and identifying underlying CKX genes. Similarly, variant CKX5 or CKX3 alleles may also be identified by phenotypically screening for number of flower, number of pods or seeds per pod or shoot or inflorescence meristem size and identifying underlying CKX3 or CKX5 genes/alleles.

The tools developed to identify a specific mutant CKX3 or CKX5 allele or the plant or plant material comprising a specific mutant CKX3 or CKX5 allele, or products which comprise plant material comprising a specific mutant CKX3 or CKX5 allele are based on the specific genomic characteristics of the specific mutant CKX3 or CKX5 allele as compared to the genomic characteristics of the corresponding wild type CKX3 or CKX5 allele, such as, a specific restriction map of the genomic region comprising the mutation region, molecular markers or the sequence of the flanking and/or mutation regions.

Once a specific mutant CKX3 or CKX5 allele has been sequenced, primers and probes can be developed which specifically recognize a sequence within the 5′ flanking, 3′ flanking and/or mutation regions of the mutant CKX3 or CKX5 allele in the nucleic acid (DNA or RNA) of a sample by way of a molecular biological technique. For instance a PCR method can be developed to identify the mutant CKX3 or CKX5 allele in biological samples (such as samples of plants, plant material or products comprising plant material). Such a PCR is based on at least two specific “primers”: one recognizing a sequence within the 5′ or 3′ flanking region of the mutant CKX3 or CKX5 allele and the other recognizing a sequence within the 3′ or 5′ flanking region of the mutant CKX3 or CKX5 allele, respectively; or one recognizing a sequence within the 5′ or 3′ flanking region of the mutant CKX3 or CKX5 allele and the other recognizing a sequence within the mutation region of the mutant CKX3 or CKX5 allele; or one recognizing a sequence within the 5′ or 3′ flanking region of the mutant CKX3 or CKX5 allele and the other recognizing a sequence spanning the joining region between the 3′ or 5′ flanking region and the mutation region of the specific mutant CKX3 or CKX5 allele (as described further below), respectively.

A suitable method for identifying a mutant CKX3 or CKX5 allele according to the invention is a method comprising subjecting the biological sample to an amplification reaction assay using a set of at least two primers, said set being selected from the group consisting of:
(a) a set of primers, wherein one of said primers specifically recognizes the 5′ or 3′ flanking region of the mutant CKX3 or CKX5 allele and the other of said primers specifically recognizes the mutation region of the mutant CKX3 or CKX5 allele, and
(b) a set of primers, wherein one of said primers specifically recognizes the 5′ or 3′ flanking region of the mutant CKX3 or CKX5 allele and the other of said primers specifically recognizes the joining region between the 3′ or 5′ flanking region and the mutation region of the mutant CKX3 or CKX5 allele, respectively.

The primers preferably have a sequence of between 15 and 35 nucleotides which under optimized PCR conditions “specifically recognize” a sequence within the 5′ or 3′ flanking region, a sequence within the mutation region, or a sequence spanning the joining region between the 3′ or 5′ flanking and mutation regions of the specific mutant CKX3 or CKX5 allele, so that a specific fragment (“mutant CKX3 or CKX5 specific fragment” or discriminating amplicon) is amplified from a nucleic acid sample comprising the specific mutant CKX3 or CKX5 allele. This means that only the targeted mutant CKX3 or CKX5 allele, and no other sequence in the plant genome, is amplified under optimized PCR conditions.

PCR primers suitable for the invention may be the following:
oligonucleotides ranging in length from 17 nt to about 200 nt, comprising a nucleotide sequence of at least 17 consecutive nucleotides, preferably 20 consecutive nucleotides selected from the 5′ or 3′ flanking sequence of a specific mutant CKX3 or CKX5 allele or the complement thereof (i.e., for example, the sequence 5′ or 3′ flanking the one or more nucleotides deleted, inserted or substituted in the mutant CKX3 or CKX5 alleles of the invention, such as the sequence 5′ or 3′ flanking the non-sense, mis-sense or frameshift mutations described above or the sequence 5′ or 3′ flanking the STOP codon mutations indicated in the above Tables or the substitution mutations indicated above or the complement thereof) (primers recognizing 5′ flanking sequences); or
oligonucleotides ranging in length from 17 nt to about 200 nt, comprising a nucleotide sequence of at least 17 consecutive nucleotides, preferably 20 nucleotides selected from the sequence of the mutation region of a specific mutant CKX3 or CKX5 allele or the complement thereof (i.e., for example, the sequence of nucleotides inserted or substituted in the CKX3 or CKX5 genes of the invention or the complement thereof) (primers recognizing mutation sequences).

The primers may of course be longer than the mentioned 17 consecutive nucleotides, and may e.g. be 18, 19, 20, 21, 30, 35, 50, 75, 100, 150, 200 nt long or even longer. The primers may entirely consist of nucleotide sequence selected from the mentioned nucleotide sequences of flanking and mutation sequences. However, the nucleotide sequence of the primers at their 5′ end (i.e. outside of the 3′-located 17 consecutive nucleotides) is less critical. Thus, the 5′ sequence of the primers may consist of a nucleotide sequence selected from the flanking or mutation sequences, as appropriate, but may contain several (e.g. 1, 2, 5, 10) mismatches. The 5′ sequence of the primers may even entirely consist of a nucleotide sequence unrelated to the flanking or mutation sequences, such as e.g. a nucleotide sequence representing restriction enzyme recognition sites. Such unrelated sequences or flanking DNA sequences with mismatches should preferably be not longer than 100, more preferably not longer than 50 or even 25 nucleotides.

Moreover, suitable primers may comprise or consist of a nucleotide sequence spanning the joining region between flanking and mutation sequences (i.e., for example, the joining region between a sequence 5′ or 3′ flanking one or more nucleotides deleted, inserted or substituted in the mutant CKX3 or CKX5 alleles of the invention and the sequence of the one or more nucleotides inserted or substituted or the sequence 3′ or 5′, respectively, flanking the one or more nucleotides deleted, such as the joining region between a sequence 5′ or 3′ flanking non-sense, missense or frameshift mutations in the CKX3 or CKX5 genes of the invention described above and the sequence of the non-sense, missense or frameshift mutations, or the joining region between a sequence 5′ or 3′ flanking a potential STOP codon mutation as indicated above or the substitution mutations indicated above and the sequence of the STOP codon mutation or the substitution mutations, respectively), provided the nucleotide sequence is not derived exclusively from either the mutation region or flanking regions.

It will also be immediately clear to the skilled artisan that properly selected PCR primer pairs should also not comprise sequences complementary to each other.

For the purpose of the invention, the “complement of a nucleotide sequence represented in SEQ ID No: X” is the nucleotide sequence which can be derived from the represented nucleotide sequence by replacing the nucleotides through their complementary nucleotide according to Chargaff s rules (A↔T; G↔C) and reading the sequence in the 5′ to 3′ direction, i.e. in opposite direction of the represented nucleotide sequence.

Examples of primers suitable to identify specific mutant CKX3 or CKX5 alleles are described in the Examples.

As used herein, “the nucleotide sequence of SEQ ID No. Z from position X to position Y” indicates the nucleotide sequence including both nucleotide endpoints.

Preferably, the amplified fragment has a length of between 50 and 1000 nucleotides, such as a length between 50 and 500 nucleotides, or a length between 100 and 350 nucleotides. The specific primers may have a sequence which is between 80 and 100% identical to a sequence within the 5′ or 3′ flanking region, to a sequence within the mutation region, or to a sequence spanning the joining region between the 3′ or 5′ flanking and mutation regions of the specific mutant CKX3 or CKX5 allele, provided the mismatches still allow specific identification of the specific mutant CKX3 or CKX5 allele with these primers under optimized PCR conditions. The range of allowable mismatches however, can easily be determined experimentally and are known to a person skilled in the art.

Detection and/or identification of a “mutant CKX3 or CKX5 specific fragment” can occur in various ways, e.g., via size estimation after gel or capillary electrophoresis or via fluorescence-based detection methods. The mutant CKX3 or CKX5 specific fragments may also be directly sequenced. Other sequence specific methods for detection of amplified DNA fragments are also known in the art.

Standard PCR protocols are described in the art, such as in “PCR Applications Manual” (Roche Molecular Biochemicals, 3rd Edition, 2006) and other references. The optimal conditions for the PCR, including the sequence of the specific primers, is specified in a “PCR identification protocol” for each specific mutant CKX3 or CKX5 allele. It is however understood that a number of parameters in the PCR identification protocol may need to be adjusted to specific laboratory conditions, and may be modified slightly to obtain similar results. For instance, use of a different method for preparation of DNA may require adjustment of, for instance, the amount of primers, polymerase, MgCl₂ concentration or annealing conditions used. Similarly, the selection of other primers may dictate other optimal conditions for the PCR identification protocol. These adjustments will however be apparent to a person skilled in the art, and are furthermore detailed in current PCR application manuals such as the one cited above.

Examples of PCR identification protocols to identify specific mutant CKX3 or CKX5 alleles are described in the Examples.

Alternatively, specific primers can be used to amplify a mutant CKX3 or CKX5 specific fragment that can be used as a “specific probe” for identifying a specific mutant CKX3 or CKX5 allele in biological samples. Contacting nucleic acid of a biological sample, with the probe, under conditions that allow hybridization of the probe with its corresponding fragment in the nucleic acid, results in the formation of a nucleic acid/probe hybrid. The formation of this hybrid can be detected (e.g. labeling of the nucleic acid or probe), whereby the formation of this hybrid indicates the presence of the specific mutant CKX3 or CKX5 allele. Such identification methods based on hybridization with a specific probe (either on a solid phase carrier or in solution) have been described in the art. The specific probe is preferably a sequence that, under optimized conditions, hybridizes specifically to a region within the 5′ or 3′ flanking region and/or within the mutation region of the specific mutant CKX3 or CKX5 allele (hereinafter referred to as “mutant CKX3 or CKX5 specific region”). Preferably, the specific probe comprises a sequence of between 10 and 1000 bp, 50 and 600 bp, between 100 to 500 bp, between 150 to 350 bp, which is at least 80%, preferably between 80 and 85%, more preferably between 85 and 90%, especially preferably between 90 and 95%, most preferably between 95% and 100% identical (or complementary) to the nucleotide sequence of a specific region. Preferably, the specific probe will comprise a sequence of about 13 to about 100 contiguous nucleotides identical (or complementary) to a specific region of the specific mutant CKX3 or CKX5 allele.

A suitable method for identifying a mutant CKX3 or CKX5 allele is a method comprising subjecting the biological sample to a hybridization assay using at least one specific probe, said probe being selected from the group consisting of:
(a) a probe specifically recognizing the mutation region of the mutant CKX3 or CKX5 allele, and
(b) a probe specifically recognizing the joining region between the 3′ or 5′ flanking region between the mutation region of the mutant CKX3 or CKX5 allele.

Specific probes suitable for the invention may be the following:
oligonucleotides ranging in length from 13 nt to about 1000 nt, comprising a nucleotide sequence of at least 13 consecutive nucleotides selected from the 5′ or 3′ flanking sequence of a specific mutant CKX3 or CKX5 allele or the complement thereof (i.e., for example, the sequence 5′ or 3′ flanking the one or more nucleotides deleted, inserted or substituted in the mutant CKX3 or CKX5 alleles of the invention, such as the sequence 5′ or 3′ flanking the non-sense, mis-sense or frameshift mutations described above or the sequence 5′ or 3′ flanking the STOP codon mutations indicated above Tables or the substitution mutations indicated above), or a sequence having at least 80% sequence identity therewith (probes recognizing 5′ flanking sequences); or
oligonucleotides ranging in length from 13 nt to about 1000 nt, comprising a nucleotide sequence of at least 13 consecutive nucleotides selected from the mutation sequence of a specific mutant CKX3 or CKX5 allele or the complement thereof (i.e., for example, the sequence of nucleotides inserted or substituted in the CKX3 or CKX5 genes of the invention, or the complement thereof), or a sequence having at least 80% sequence identity therewith (probes recognizing mutation sequences).

The probes may entirely consist of nucleotide sequence selected from the mentioned nucleotide sequences of flanking and mutation sequences. However, the nucleotide sequence of the probes at their 5′ or 3′ ends is less critical. Thus, the 5′ or 3′ sequences of the probes may consist of a nucleotide sequence selected from the flanking or mutation sequences, as appropriate, but may consist of a nucleotide sequence unrelated to the flanking or mutation sequences. Such unrelated sequences should preferably be not longer than 50, more preferably not longer than 25 or even not longer than 20 or 15 nucleotides.

Moreover, suitable probes may comprise or consist of a nucleotide sequence spanning the joining region between flanking and mutation sequences (i.e., for example, the joining region between a sequence 5′ or 3′ flanking one or more nucleotides deleted, inserted or substituted in the mutant CKX3 or CKX5 alleles of the invention and the sequence of the one or more nucleotides inserted or substituted or the sequence 3′ or 5′, respectively, flanking the one or more nucleotides deleted, such as the joining region between a sequence 5′ or 3′ flanking non-sense, mis-sense or frameshift mutations in the CKX3 or CKX5 genes of the invention described above and the sequence of the non-sense, mis-sense or frameshift mutations, or the joining region between a sequence 5′ or 3′ flanking a potential STOP codon mutation as indicated in the above Tables or the substitution mutations indicated above and the sequence of the potential STOP codon or substitution mutation, respectively), provided the mentioned nucleotide sequence is not derived exclusively from either the mutation region or flanking regions.

Examples of specific probes suitable to identify specific mutant CKX3 or CKX5 alleles are described in the Examples.

Detection and/or identification of a “mutant CKX3 or CKX5 specific region” hybridizing to a specific probe can occur in various ways, e.g., via size estimation after gel electrophoresis or via fluorescence-based detection methods. Other sequence specific methods for detection of a “mutant CKX3 or CKX5 specific region” hybridizing to a specific probe are also known in the art.

Alternatively, plants or plant parts comprising one or more mutant ckx5 or ckx5 and ckx3 alleles can be generated and identified using other methods, such as the “Delete-a-gene™” method which uses PCR to screen for deletion mutants generated by fast neutron mutagenesis (reviewed by Li and Zhang, 2002, Funct Integr Genomics 2:254-258), by the TILLING (Targeting Induced Local Lesions IN Genomes) method which identifies EMS-induced point mutations using denaturing high-performance liquid chromatography (DHPLC) to detect base pair changes by heteroduplex analysis (McCallum et al., 2000, Nat Biotech 18:455, and McCallum et al. 2000, Plant Physiol. 123, 439-442), etc. As mentioned, TILLING uses high-throughput screening for mutations (e.g. using Cell cleavage of mutant-wildtype DNA heteroduplexes and detection using a sequencing gel system). Thus, the use of TILLING to identify plants or plant parts comprising one or more mutant ckx5 or ckx3 alleles and methods for generating and identifying such plants, plant organs, tissues and seeds is encompassed herein. Thus in one embodiment, the method according to the invention comprises the steps of mutagenizing plant seeds (e.g. EMS mutagenesis), pooling of plant individuals or DNA, PCR amplification of a region of interest, heteroduplex formation and high-throughput detection, identification of the mutant plant, sequencing of the mutant PCR product. It is understood that other mutagenesis and selection methods may equally be used to generate such mutant plants.

Instead of inducing mutations in CKX3 or CKX5 alleles, natural (spontaneous) mutant alleles may be identified by methods known in the art. For example, ECOTILLING may be used (Henikoff et al. 2004, Plant Physiology 135(2):630-6) to screen a plurality of plants or plant parts for the presence of natural mutant ckx3/ckx5 alleles. As for the mutagenesis techniques above, preferably Brassica species are screened which comprise an A and/or a C genome, so that the identified ckx3/ckx5 allele can subsequently be introduced into other Brassica species, such as Brassica napus, by crossing (inter- or intraspecific crosses) and selection. In ECOTILLING natural polymorphisms in breeding lines or related species are screened for by the TILLING methodology described above, in which individual or pools of plants are used for PCR amplification of the ckx3/ckx5 target, heteroduplex formation and high-throughput analysis. This can be followed by selecting individual plants having a required mutation that can be used subsequently in a breeding program to incorporate the desired mutant allele.

The identified mutant alleles can then be sequenced and the sequence can be compared to the wild type allele to identify the mutation(s). Optionally functionality can be tested as indicated above. Using this approach a plurality of mutant ckx3/ckx5 alleles (and Brassica plants comprising one or more of these) can be identified. The desired mutant alleles can then be combined with the desired wild type alleles by crossing and selection methods as described further below. Finally a single plant comprising the desired number of mutant ckx3/ckx5 and the desired number of wild type CKX3 or CKX5 alleles is generated.

Oligonucleotides suitable as PCR primers or specific probes for detection of a specific mutant CKX3 or CKX5 allele can also be used to develop methods to determine the zygosity status of the specific mutant CKX3 or CKX5 allele.

To determine the zygosity status of a specific mutant CKX3 or CKX5 allele, a PCR-based assay can be developed to determine the presence of a mutant and/or corresponding wild type CKX3 or CKX5 specific allele.

To determine the zygosity status of a specific mutant CKX3 or CKX5 allele, two primers specifically recognizing the wild-type CKX3 or CKX5 allele can be designed in such a way that they are directed towards each other and have the mutation region located in between the primers. These primers may be primers specifically recognizing the 5′ and 3′ flanking sequences, respectively. This set of primers allows simultaneous diagnostic PCR amplification of the mutant, as well as of the corresponding wild type CKX3 or CKX5 allele.

Alternatively, to determine the zygosity status of a specific mutant CKX3 or CKX5 allele, two primers specifically recognizing the wild-type CKX3 or CKX5 allele can be designed in such a way that they are directed towards each other and that one of them specifically recognizes the mutation region. These primers may be primers specifically recognizing the sequence of the 5′ or 3′ flanking region and the mutation region of the wild type CKX3 or CKX5 allele, respectively. This set of primers, together with a third primer which specifically recognizes the sequence of the mutation region in the mutant CKX3 or CKX5 allele, allow simultaneous diagnostic PCR amplification of the mutant CKX3 or CKX5 gene, as well as of the wild type CKX3 or CKX5 gene.

Alternatively, to determine the zygosity status of a specific mutant CKX3 or CKX5 allele, two primers specifically recognizing the wild-type CKX3 or CKX5 allele can be designed in such a way that they are directed towards each other and that one of them specifically recognizes the joining region between the 5′ or 3′ flanking region and the mutation region. These primers may be primers specifically recognizing the 5′ or 3′ flanking sequence and the joining region between the mutation region and the 3′ or 5′ flanking region of the wild type CKX3 or CKX5 allele, respectively. This set of primers, together with a third primer which specifically recognizes the joining region between the mutation region and the 3′ or 5′ flanking region of the mutant CKX3 or CKX5 allele, respectively, allow simultaneous diagnostic PCR amplification of the mutant CKX3 or CKX5 gene, as well as of the wild type CKX3 or CKX5 gene.

Alternatively, the zygosity status of a specific mutant CKX3 or CKX5 allele can be determined by using alternative primer sets that specifically recognize mutant and wild type CKX3 or CKX5 alleles.

A suitable method for determining the zygosity status of a mutant CKX3 or CKX5 allele comprises subjecting the genomic DNA of said plant, or a cell, part, seed or progeny thereof, to an amplification reaction using a set of at least two or at least three primers, wherein at least two of said primers specifically recognize the wild type CKX3 or CKX5 allele, said at least two primers being selected from the group consisting of:
(a) a first primer which specifically recognizes the 5′ or 3′ flanking region of the mutant and the wild type CKX3 or CKX5 allele, and a second primer which specifically recognizes the mutation region of the wild type CKX3 or CKX5 allele, and
(b) a first primer which specifically recognizes the 5′ or 3′ flanking region of the mutant and the wild type CKX3 or CKX5 allele, and a second primer which specifically recognizes the joining region between the 3′ or 5′ flanking region and the mutation region of the wild type CKX3 or CKX5 allele, respectively, and
   wherein at least two of said primers specifically recognize the mutant CKX3 or CKX5 allele, said at least two primers being selected from the group consisting of:
(a) the first primer which specifically recognizes the 5′ or 3′ flanking region of the mutant and the wild type CKX3 or CKX5 allele, and a third primer which specifically recognizes the mutation region of the mutant CKX3 or CKX5 allele, and
(b) the first primer which specifically recognizes the 5′ or 3′ flanking region of the mutant and the wild type CKX3 or CKX5 allele, and a third primer which specifically recognizes the joining region between the 3′ or 5′ flanking region and the mutation region of the mutant CKX3 or CKX5 allele, respectively.

If the plant is homozygous for the mutant CKX3 or CKX5 gene or the corresponding wild type CKX3 or CKX5 gene, the diagnostic PCR assays described above will give rise to a single PCR product typical, preferably typical in length, for either the mutant or wild type CKX3 or CKX5 allele. If the plant is heterozygous for the mutant CKX3 or CKX5 allele, two specific PCR products will appear, reflecting both the amplification of the mutant and the wild type CKX3 or CKX5 allele.

Identification of the wild type and mutant CKX3 or CKX5 specific PCR products can occur e.g. by size estimation after gel or capillary electrophoresis (e.g. for mutant CKX3 or CKX5 alleles comprising a number of inserted or deleted nucleotides which results in a size difference between the fragments amplified from the wild type and the mutant CKX3 or CKX5 allele, such that said fragments can be visibly separated on a gel); by evaluating the presence or absence of the two different fragments after gel or capillary electrophoresis, whereby the diagnostic PCR amplification of the mutant CKX3 or CKX5 allele can, optionally, be performed separately from the diagnostic PCR amplification of the wild type CKX3 or CKX5 allele; by direct sequencing of the amplified fragments; or by fluorescence-based detection methods.

Examples of primers suitable to determine the zygosity of specific mutant CKX3 or CKX5 alleles are described in the Examples.

Alternatively, to determine the zygosity status of a specific mutant CKX3 or CKX5 allele, a hybridization-based assay can be developed to determine the presence of a mutant and/or corresponding wild type CKX3 or CKX5 specific allele:

To determine the zygosity status of a specific mutant CKX3 or CKX5 allele, two specific probes recognizing the wild-type CKX3 or CKX5 allele can be designed in such a way that each probe specifically recognizes a sequence within the CKX3 or CKX5 wild type allele and that the mutation region is located in between the sequences recognized by the probes. These probes may be probes specifically recognizing the 5′ and 3′ flanking sequences, respectively. The use of one or, preferably, both of these probes allows simultaneous diagnostic hybridization of the mutant, as well as of the corresponding wild type CKX3 or CKX5 allele.

Alternatively, to determine the zygosity status of a specific mutant CKX3 or CKX5 allele, two specific probes recognizing the wild-type CKX3 or CKX5 allele can be designed in such a way that one of them specifically recognizes a sequence within the CKX3 or CKX5 wild type allele upstream or downstream of the mutation region, preferably upstream of the mutation region, and that one of them specifically recognizes the mutation region. These probes may be probes specifically recognizing the sequence of the 5′ or 3′ flanking region, preferably the 5′ flanking region, and the mutation region of the wild type CKX3 or CKX5 allele, respectively. The use of one or, preferably, both of these probes, optionally, together with a third probe which specifically recognizes the sequence of the mutation region in the mutant CKX3 or CKX5 allele, allow diagnostic hybridization of the mutant and of the wild type CKX3 or CKX5 gene.

Alternatively, to determine the zygosity status of a specific mutant CKX3 or CKX5 allele, a specific probe recognizing the wild-type CKX3 or CKX5 allele can be designed in such a way that the probe specifically recognizes the joining region between the 5′ or 3′ flanking region, preferably the 5′ flanking region, and the mutation region of the wild type CKX3 or CKX5 allele. This probe, optionally, together with a second probe that specifically recognizes the joining region between the 5′ or 3′ flanking region, preferably the 5′ flanking region, and the mutation region of the mutant CKX3 or CKX5 allele, allows diagnostic hybridization of the mutant and of the wild type CKX3 or CKX5 gene.

Alternatively, the zygosity status of a specific mutant CKX3 or CKX5 allele can be determined by using alternative sets of probes that specifically recognize mutant and wild type CKX3 or CKX5 alleles.

A suitable method for determining the zygosity status of a mutant CKX3 or CKX5 allele comprises subjecting the genomic DNA of said plant, or a cell, part, seed or progeny thereof, to a hybridization assay using a set of at least two specific probes, wherein at least one of said specific probes specifically recognizes the wild type CKX3 or CKX5 allele, said at least one probe selected from the group consisting of:
(a) a first probe which specifically recognizes the 5′ or 3′ flanking region of the mutant and the wild type CKX3 or CKX5 allele, and a second probe which specifically recognizes the mutation region of the wild type CKX3 or CKX5 allele,
(b) a first probe which specifically recognizes the 5′ or 3′ flanking region of the mutant and the wild type CKX3 or CKX5 allele, and a second probe which specifically recognizes the joining region between the 3′ or 5′ flanking region and the mutation region of the wild type CKX3 or CKX5 allele, respectively, and
(c) a probe which specifically recognizes the joining region between the 5′ or 3′ flanking region and the mutation region of the wild type CKX3 or CKX5 allele, and
   wherein at least one of said specific probes specifically recognize(s) the mutant CKX3 or CKX5 allele, said at least one probe selected from the group consisting of:
(a) the first probe which specifically recognizes the 5′ or 3′ flanking region of the mutant and the wild type CKX3 or CKX5 allele, and a third probe which specifically recognizes the mutation region of the mutant CKX3 or CKX5 allele,
(b) the first probe which specifically recognizes the 5′ or 3′ flanking region of the mutant and the wild type CKX3 or CKX5 allele, and a third probe which specifically recognizes the joining region between the 5′ or 3′ flanking region and the mutation region of the mutant CKX3 or CKX5 allele, and
(c) a probe which specifically recognizes the joining region between the 5′ or 3′ flanking region and the mutation region of the mutant CKX3 or CKX5 allele.

If the plant is homozygous for the mutant CKX3 or CKX5 gene or the corresponding wild type CKX3 or CKX5 gene, the diagnostic hybridization assays described above will give rise to a single specific hybridization product, such as one or more hybridizing DNA (restriction) fragments, typical, preferably typical in length, for either the mutant or wild type CKX3 or CKX5 allele. If the plant is heterozygous for the mutant CKX3 or CKX5 allele, two specific hybridization products will appear, reflecting both the hybridization of the mutant and the wild type CKX3 or CKX5 allele.

Identification of the wild type and mutant CKX3 or CKX5 specific hybridization products can occur e.g. by size estimation after gel or capillary electrophoresis (e.g. for mutant CKX3 or CKX5 alleles comprising a number of inserted or deleted nucleotides which results in a size difference between the hybridizing DNA (restriction) fragments from the wild type and the mutant CKX3 or CKX5 allele, such that said fragments can be visibly separated on a gel); by evaluating the presence or absence of the two different specific hybridization products after gel or capillary electrophoresis, whereby the diagnostic hybridization of the mutant CKX3 or CKX5 allele can, optionally, be performed separately from the diagnostic hybridization of the wild type CKX3 or CKX5 allele; by direct sequencing of the hybridizing DNA (restriction) fragments; or by fluorescence-based detection methods.

Examples of probes suitable to determine the zygosity of specific mutant CKX3 or CKX5 alleles are described in the Examples.

Furthermore, detection methods specific for a specific mutant CKX3 or CKX5 allele that differ from PCR- or hybridization-based amplification methods can also be developed using the specific mutant CKX3 or CKX5 allele specific sequence information provided herein. Such alternative detection methods include linear signal amplification detection methods based on invasive cleavage of particular nucleic acid structures, also known as Invader™ technology, (as described e.g. in U.S. Pat. No. 5,985,557 “Invasive Cleavage of Nucleic Acids”, U.S. Pat. No. 6,001,567 “Detection of Nucleic Acid sequences by Invader Directed Cleavage, or Lyamichev et al., 1999, Nature Biotechnology 17: 292, incorporated herein by reference), RT-PCR-based detection methods, such as Taqman, or other detection methods, such as SNPlex. Briefly, in the Invader™ technology, the target mutation sequence may e.g. be hybridized with a labeled first nucleic acid oligonucleotide comprising the nucleotide sequence of the mutation sequence or a sequence spanning the joining region between the 3′ flanking region and the mutation region, and with a second nucleic acid oligonucleotide comprising the 5′ flanking sequence immediately downstream and adjacent to the mutation sequence, wherein the first and second oligonucleotide overlap by at least one nucleotide. Further, the target mutation sequence may e.g. be hybridized with a labeled first nucleic acid oligonucleotide complementary to the nucleotide sequence of the mutation sequence or a sequence spanning the joining region between the 5′ flanking region and the mutation region, and with a second nucleic acid oligonucleotide complementary to the 3′ flanking sequence immediately downstream and adjacent to the mutation sequence, wherein the first and second oligonucleotide overlap by at least one nucleotide. The duplex or triplex structure that is produced by this hybridization allows selective probe cleavage with an enzyme (Cleavase®) leaving the target sequence intact. The cleaved labeled probe is subsequently detected, potentially via an intermediate step resulting in further signal amplification. In a further embodiment, the first nucleic acid oligonucleotide comprises at its 5′ end a 5′ flap which is not complementary or corresponding to target mutant or wild type sequences, and immediately downstream of the flap the joining region between the 3′ flanking region and the mutation region, wherein the mutation sequence is at the 5′ end of said joining region, and said second nucleic acid oligonucleotide comprises the 5′ flanking sequence immediately upstream of and contiguous with the mutation region, and at its 3′ end immediately downstream of the 5′ flanking sequence one additional nucleotide which may be any nucleotide. In another embodiment, the first nucleic acid oligonucleotide comprises at its 5′ end a 5′ flap which is not complementary or corresponding to target mutant or wild type sequences, and immediately downstream of the flap the sequence complementary to the joining region between the 5′ flanking region and the mutation region, wherein complementary of the mutation sequence is at the 5′ end of said joining region, and said second nucleic acid oligonucleotide complementary to the 3′ flanking sequence immediately upstream of and contiguous with the mutation region, and at its 3′ end immediately downstream of the complement to the 3′ flanking sequence one additional nucleotide which may be any nucleotide. The length of the sequence corresponding to, or complementary to, the joining region in the first oligonucleotide may be at least 5, or at least 8, or at least 10, or at least 15, or at least 20, or at least 25, or at least 30, or at least 40, or at least 50 nucleotides. The length of the sequence corresponding to, or complementary to the flanking sequence in the second oligonucleotide may be at least 5, or at least 8, or at least 10, or at least 15, or at least 20, or at least 25, or at least 30, or at least 40, or at least 50 nucleotides. The length of the 5′ flap of the first oligonucleotide may be at least 3, or at least 5, or at least 8, or at least 10, or at least 15, or at least 20 nucleotides.

A suitable method for identifying a mutant CKX3 or CKX5 allele is a method comprising subjecting the biological sample to a hybridization assay with
(a) a labelled first nucleic acid oligonucleotide, said first nucleic acid oligonucleotide comprising the nucleotide sequence of the mutation sequence or a sequence spanning the joining region between the 3′ flanking region and the mutation region, and a second nucleic acid oligonucleotide comprising the 5′ flanking sequence immediately downstream and adjacent to the mutation sequence, and wherein the first and second oligonucleotide overlap by at least one nucleotide; or
(b) a labelled first nucleic acid oligonucleotide, said first nucleic acid oligonucleotide complementary to the nucleotide sequence of the mutation sequence or a sequence spanning the joining region between the 5′ flanking region and the mutation region, and a second nucleic acid oligonucleotide complementary to the 3′ flanking sequence immediately downstream and adjacent to the mutation sequence, and wherein the first and second oligonucleotide overlap by at least one nucleotide.

Mutant CKX3 or CKX5 alleles can also be identified by determining the sequence of the CKX3 or CKX5 alleles. Sequencing can be performed by methods known in the art.

A “kit”, as used herein, refers to a set of reagents for the purpose of performing the method of the invention, more particularly, the identification of a specific mutant CKX3 or CKX5 allele in biological samples or the determination of the zygosity status of plant material comprising a specific mutant CKX3 or

CKX5 allele. More particularly, a preferred embodiment of the kit of the invention comprises at least two specific primers, as described above, for identification of a specific mutant CKX3 or CKX5 allele, or at least two or three specific primers for the determination of the zygosity status. Optionally, the kit can further comprise any other reagent described herein in the PCR identification protocol. Alternatively, according to another embodiment of this invention, the kit can comprise at least one specific probe, which specifically hybridizes with nucleic acid of biological samples to identify the presence of a specific mutant CKX3 or CKX5 allele therein, as described above, for identification of a specific mutant CKX3 or CKX5 allele, or at least two or three specific probes for the determination of the zygosity status. Optionally, the kit can further comprise any other reagent (such as but not limited to hybridizing buffer, amplification buffer, label) for identification of a specific mutant CKX3 or CKX5 allele in biological samples, using the specific probe.

The kit of the invention can be used, and its components can be specifically adjusted, for purposes of quality control (e.g., purity of seed lots), detection of the presence or absence of a specific mutant CKX3 or CKX5 allele in plant material or material comprising or derived from plant material, such as but not limited to food or feed products.

The term “primer” as used herein encompasses any nucleic acid that is capable of priming the synthesis of a nascent nucleic acid in a template-dependent process, such as PCR. Typically, primers are oligonucleotides from 10 to 30 nucleotides, but longer sequences can be employed. Primers may be provided in double-stranded form, though the single-stranded form is preferred. Probes can be used as primers, but are designed to bind to the target DNA or RNA and need not be used in an amplification process.

The term “recognizing” as used herein when referring to specific primers, refers to the fact that the specific primers specifically hybridize to a nucleic acid sequence in a specific mutant CKX3 or CKX5 allele under the conditions set forth in the method (such as the conditions of the PCR identification protocol), whereby the specificity is determined by the presence of positive and negative controls.

The term “hybridizing”, as used herein when referring to specific probes, refers to the fact that the probe binds to a specific region in the nucleic acid sequence of a specific mutant CKX3 or CKX5 allele under standard stringency conditions. Standard stringency conditions as used herein refers to the conditions for hybridization described herein or to the conventional hybridizing conditions as described by Sambrook et al., 1989 (Molecular Cloning: A Laboratory Manual, Second Edition, Cold Spring Harbour Laboratory Press, NY) which for instance can comprise the following steps: 1) immobilizing plant genomic DNA fragments or BAC library DNA on a filter, 2) prehybridizing the filter for 1 to 2 hours at 65° C. in 6×SSC, 5× Denhardt's reagent, 0.5% SDS and 20 μg/ml denaturated carrier DNA, 3) adding the hybridization probe which has been labeled, 4) incubating for 16 to 24 hours, 5) washing the filter once for 30 min. at 68° C. in 6×SSC, 0.1% SDS, 6) washing the filter three times (two times for 30 min. in 30m1 and once for 10 min in 500 ml) at 68° C. in 2×SSC, 0.1% SDS, and 7) exposing the filter for 4 to 48 hours to X-ray film at −70° C.

As used in herein, a “biological sample” is a sample of a plant, plant material or product comprising plant material. Preferably, the biological sample contains nucleic acids such as DNA or RNA. The term “plant” is intended to encompass plant tissues, at any stage of maturity, as well as any cells, tissues, or organs taken from or derived from any such plant, including without limitation, any seeds, leaves, stems, flowers, roots, single cells, gametes, cell cultures, tissue cultures or protoplasts. “Plant material”, as used herein refers to material that is obtained or derived from a plant. Products comprising plant material relate to food, feed or other products that are produced using plant material or can be contaminated by plant material. It is understood that, in the context of the present invention, such biological samples are tested for the presence of nucleic acids specific for a specific mutant CKX3 or CKX5 allele, implying the presence of nucleic acids in the samples. Thus the methods referred to herein for identifying a specific mutant CKX3 or CKX5 allele in biological samples, relate to the identification in biological samples of nucleic acids that comprise the specific mutant CKX3 or CKX5 allele.

The present invention also relates to the combination of specific CKX3 or CKX5 alleles in one plant, to the transfer of one or more specific mutant CKX3 or CKX5 allele(s) from one plant to another plant, to the plants comprising one or more specific mutant CKX3 or CKX5 allele(s), the progeny obtained from these plants and to plant cells, plant parts, and plant seeds derived from these plants.

Thus, in one embodiment of the invention a method for combining two or more selected mutant CKX3 or CKX5 alleles in one plant is provided comprising the steps of:
(a) generating and/or identifying two or more plants each comprising one or more selected mutant CKX3 or CKX5 alleles, as described above,
(b) crossing a first plant comprising one or more selected mutant CKX3 or CKX5 alleles with a second plant comprising one or more other selected mutant CKX3 or CKX5 alleles, collecting F1 seeds from the cross, and, optionally, identifying an F1 plant comprising one or more selected mutant CKX3 or CKX5 alleles from the first plant with one or more selected mutant CKX3 or CKX5 alleles from the second plant, as described above,
(c) optionally, repeating step (b) until an F1 plant comprising all selected mutant CKX5 or CKX5 and CKX3 alleles is obtained,
(d) optionally,
   identifying an F 1 plant, which is homozygous or heterozygous for a selected mutant CKX3 or CKX5 allele by determining the zygosity status of the mutant CKX3 or CKX5 alleles, as described above, or
   generating plants which are homozygous for one or more of the selected mutant CKX3 or CKX5 alleles by performing one of the following steps:
      extracting doubled haploid plants from treated microspore or pollen cells of F 1 plants comprising the one or more selected mutant CKX3 or CKX5 alleles, as described above,
      selfing the F1 plants comprising the one or more selected mutant CKX3 or CKX5 allele(s) for one or more generations (y), collecting F1 Sy seeds from the selfings, and identifying F1 Sy plants, which are homozygous for the one or more mutant CKX3 or CKX5 allele, as described above.

In another embodiment of the invention a method for transferring one or more mutant CKX3 or CKX5 alleles from one plant to another plant is provided comprising the steps of:
(a) generating and/or identifying a first plant comprising one or more selected mutant CKX3 or CKX5 alleles, as described above, or generating the first plant by combining the one or more selected mutant CKX3 or CKX5 alleles in one plant, as described above (wherein the first plant is homozygous or heterozygous for the one or more mutant CKX3 or CKX5 alleles),
(b) crossing the first plant comprising the one or more mutant CKX3 or CKX5 alleles with a second plant not comprising the one or more mutant CKX3 or CKX5 alleles, collecting F1 seeds from the cross (wherein the seeds are heterozygous for a mutant CKX3 or CKX5 allele if the first plant was homozygous for that mutant CKX3 or CKX5 allele, and wherein half of the seeds are heterozygous and half of the seeds are azygous for, i.e. do not comprise, a mutant CKX3 or CKX5 allele if the first plant was heterozygous for that mutant CKX3 or CKX5 allele), and, optionally, identifying F1 plants comprising one or more selected mutant CKX3 or CKX5 alleles, as described above,
(c) backcrossing F1 plants comprising one or more selected mutant CKX3 or CKX5 alleles with the second plant not comprising the one or more selected mutant CKX3 or CKX5 alleles for one or more generations (x), collecting BCx seeds from the crosses, and identifying in every generation BCx plants comprising the one or more selected mutant CKX3 or CKX5 alleles, as described above,
(d) optionally, generating BCx plants which are homozygous for the one or more selected mutant CKX3 or CKX5 alleles by performing one of the following steps:
   extracting doubled haploid plants from treated microspore or pollen cells of BCx plants comprising the one or more desired mutant CKX3 or CKX5 allele(s), as described above,
   selfing the BCx plants comprising the one or more desired mutant CKX3 or CKX5 allele(s) for one or more generations (y), collecting BCx Sy seeds from the selfings, and identifying BCx Sy plants, which are homozygous for the one or more desired mutant CKX3 or CKX5 allele, as described above.

Said method for transferring one or more mutant CKX3 or CKX5 alleles from one plant to another is also suitable for combining one or more mutant CKX3 or CKX5 alleles in one plant, said method for combining at least two selected mutant CKX3 or CKX5 alleles comprising the steps of:
(a) identifying at least two plants each comprising at least one selected mutant CKX3 or CKX5 allele,
(b) crossing the at least two plants and collecting F1 hybrid seeds from the at least one cross, and
(c) optionally, identifying an F1 plant comprising at least two selected mutant CKX3 or CKX5 alleles.

Said plants comprising said at least one selected mutant CKX3 or CKX5 alleles can be identified using the methods as described herein.

In one aspect of the invention, the first and the second plant are Brassicaceae plants, particularly Brassica plants, especially Brassica napus plants or plants from another Brassica crop species. In another aspect of the invention, the first plant is a Brassicaceae plant, particularly a Brassica plant, especially a Brassica napus plant or a plant from another Brassica crop species, and the second plant is a plant from a Brassicaceae breeding line, particularly from a Brassica breeding line, especially from a Brassica napus breeding line or from a breeding line from another Brassica crop species. “Breeding line”, as used herein, is a preferably homozygous plant line distinguishable from other plant lines by a preferred genotype and/or phenotype that is used to produce hybrid offspring.

In yet another embodiment of the invention, a method for making a plant, in particular a Brassica crop plant, such as a Brassica napus plant, of which the number of flowers, the numbers or pods or the TSW is increased is provided comprising combining and/or transferring mutant CKX5 or CKX5 and CKX3 alleles according to the invention in or to one Brassica plant, as described above.

Also provided herein is a method to increase the number of flowers, the numbers or pods or the TSW, comprising introducing at least one mutant CKX5 allele or at least one mutant CKX5 and at least one mutant CKX3 allele into a Brassica plant, or comprising introducing the chimeric gene according to the invention in a Brassica plant.

The mutant CKX3 or CKX5 alleles can be introduced into said Brassica plants using methods as described herein comprising combining and/or transferring mutant CKX3 or CKX5 alleles according to the invention in or to one Brassica plant. The mutant CKX3 or CKX5 allele can also be introduced through, e.g. mutagenesis or gene targeting. Said method can further comprise identification of the presence of the mutant CKX3 or CKX5 alleles using methods as described herein.

The chimeric gene according to the invention can be introduced into Brassica plants using transformation.

A method to increase the number of flowers, the numbers of pods or the TSW may comprise
(a) providing plant cells with one or more chimeric genes to create transgenic plant cells, said chimeric genes comprising the following operably linked DNA fragments
   i. a plant-expressible promoter;
   ii. a DNA region, which when transcribed yields an RNA or protein molecule inhibitory to the expression or protein activity of one or more CKX5 genes/proteins or two CKX5 and one or more CKX3 genes or proteins; and, optionally,
   iii. a 3′ end region involved in transcription termination and polyadenylation;
(b) regenerating a population of transgenic plant lines from said transgenic plant cell; and
(c) identifying a plant line with increased number of flowers within said population of transgenic plant lines.

Means for preparing chimeric genes are well known in the art. Methods for making chimeric genes and vectors comprising such chimeric genes particularly suited to plant transformation are described in U.S. Pat. Nos. 4,971,908, 4,940,835, 4,769,061 and 4,757,011. The chimeric gene may also contain one or more additional nucleic acid sequences.

Said chimeric gene may be introduced in said Brassica plant by transformation. The term “transformation” herein refers to the introduction (or transfer) of nucleic acid into a recipient host such as a plant or any plant parts or tissues including plant cells, protoplasts, calli, roots, tubers, seeds, stems, leaves, seedlings, embryos and pollen. Plants containing the transformed nucleic acid sequence are referred to as “transgenic plants”. Transformed, transgenic and recombinant refer to a host organism such as a plant into which a heterologous nucleic acid molecule (e.g. an expression cassette or a recombinant vector) has been introduced. The transformed nucleic acid can be stably integrated into the genome of the plant.

As used herein, the phrase “transgenic plant” refers to a plant having an transformed nucleic acid stably introduced into a genome of the plant, for example, the nuclear or plastid genomes. In other words, plants containing transformed nucleic acid sequence are referred to as “transgenic plants”. Transgenic and recombinant refer to a host organism such as a plant into which a heterologous nucleic acid molecule (e.g. the promoter, the chimeric gene or the vector as described herein) has been introduced. The nucleic acid can be stably integrated into the genome of the plant.

Transformation methods are well known in the art and include Agrobacterium-mediated transformation. Agrobacterium-mediated transformation of cotton has been described e.g. in U.S. Pat. No. 5,004,863, in U.S. Pat. No. 6,483,013 and WO2000/71733. Plants may also be transformed by particle bombardment: Particles of gold or tungsten are coated with DNA and then shot into young plant cells or plant embryos. This method also allows transformation of plant plastids. Viral transformation (transduction) may be used for transient or stable expression of a gene, depending on the nature of the virus genome. The desired genetic material is packaged into a suitable plant virus and the modified virus is allowed to infect the plant. The progeny of the infected plants is virus free and also free of the inserted gene. Suitable methods for viral transformation are described or further detailed e. g. in WO 90/12107, WO 03/052108 or WO 2005/098004. Further suitable methods well-known in the art are microinjection, electroporation of intact cells, polyethyleneglycol-mediated protoplast transformation, electroporation of protoplasts, liposome-mediated transformation, silicon-whiskers mediated transformation etc. Said transgene may be stably integrated into the genome of said plant cell, resulting in a transformed plant cell. The transformed plant cells obtained in this way may then be regenerated into mature fertile transformed plants.

In one aspect of the invention, the plant according to the invention is a Brassica plant comprising at least one CKX5 gene at least two CKX3 genes wherein increase in number of flowers or increase in TSW is increased by combining and/or transferring six mutant alleles according to the invention in or to the Brassica plant, as described above (four CKX3 and two CKX5 alleles).

In still another embodiment of the invention, a method for making a hybrid Brassica crop seed or plant comprising at least two CKX5 and at least four CKX3 genes, in particular a hybrid Brassica napus seed or plant, of which the number of flowers or TSW is increased is provided, comprising the steps of:
(a) generating and/or identifying a first plant comprising a first and a second selected mutant CKX5 allele in homozygous state and a second plant comprising at least one selected mutant CKX allele in homozygous state, as described above,
(b) crossing the first and the second plant and collecting F1 hybrid seeds from the cross.

In one aspect of the invention, the first or the second selected mutant CKX5 allele may be the same mutant CKX5 allele as the third selected mutant CKX3 or CKX5 allele, such that the F1 hybrid seeds are homozygous for one mutant CKX5 allele and heterozygous for the other. In another aspect of the invention, the first plant is used as a male parent plant and the second plant is used as a female parent plant.

Whenever reference to a “plant” or “plants” according to the invention is made, it is understood that also plant parts (cells, tissues or organs, seed pods, seeds, severed parts such as roots, leaves, flowers, pollen, etc.), progeny of the plants which retain the distinguishing characteristics of the parents (especially the fruit dehiscence properties), such as seed obtained by selfing or crossing, e.g. hybrid seed (obtained by crossing two inbred parental lines), hybrid plants and plant parts derived there from are encompassed herein, unless otherwise indicated.

In some embodiments, the plant cells of the invention, i.e. a plant cell comprising at least one mutant CKX5 or at least one mutant CKX3 or CKX5 allele, or a plant cell wherein expression of at least one CKX5 or at least one CKX5 and one CKX3 gene is reduced, as well as plant cells generated according to the methods of the invention, may be non-propagating cells.

The obtained plants according to the invention can be used in a conventional breeding scheme to produce more plants with the same characteristics or to introduce the characteristic of the presence of at least one mutant CKX5 allele, having reduced expression of at least one CKX5 in other varieties of the same or related plant species, or in hybrid plants. The obtained plants can further be used for creating propagating material. Plants according to the invention can further be used to produce gametes, seeds (including crushed seeds and seed cakes), seed oil, embryos, either zygotic or somatic, progeny or hybrids of plants obtained by methods of the invention. Seeds obtained from the plants according to the invention are also encompassed by the invention.

“Creating propagating material”, as used herein, relates to any means know in the art to produce further plants, plant parts or seeds and includes inter alia vegetative reproduction methods (e.g. air or ground layering, division, (bud) grafting, micropropagation, stolons or runners, storage organs such as bulbs, corms, tubers and rhizomes, striking or cutting, twin-scaling), sexual reproduction (crossing with another plant) and asexual reproduction (e.g. apomixis, somatic hybridization).

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a nucleic acid which is functionally or structurally defined, may comprise additional DNA regions etc.

The sequence listing contained in the file named “BCS15-2012_ST25.txt”, which is 142 kilobytes (size as measured in Microsoft Windows®), contains 45 sequences SEQ ID NO: 1 through SEQ ID NO: 45 is filed herewith by electronic submission and is incorporated by reference herein.

In the description and examples, reference is made to the following sequences:

### Sequences

SEQ ID No.1: Arabidopsis thaliana CKX3 genomic sequence At5g56970

SEQ ID No.2: Arabidopsis thaliana CKX3 cDNA sequence (coding sequence)

SEQ ID No.3: Arabidopsis thaliana CKX3 amino acid sequence

SEQ ID No.4: Arabidopsis thaliana CKX5 genomic sequence At1g75450

SEQ ID No.5: Arabidopsis thaliana CKX5 cDNA sequence (coding sequence)

SEQ ID No.6: Arabidopsis thaliana CKX5 amino acid sequence

SEQ ID No.7: Brassica napus CKX3-A1 genomic sequence

SEQ ID No.8: Brassica napus CKX3-A1 cDNA sequence (coding sequence)

SEQ ID No.9: Brassica napus CKX3-A1 amino acid sequence

SEQ ID No.10: Brassica napus CKX3-A2 genomic sequence

SEQ ID No.11: Brassica napus CKX3-A2 cDNA sequence (coding sequence)

SEQ ID No.12: Brassica napus CKX3-A2 amino acid sequence

SEQ ID No.13: Brassica napus CKX3-C1 genomic sequence

SEQ ID No.14: Brassica napus CKX3-C1 cDNA sequence (coding sequence)

SEQ ID No.15: Brassica napus CKX3-C1 amino acid sequence

SEQ ID No.16: Brassica napus CKX3-C2 genomic sequence

SEQ ID No.17: Brassica napus CKX3-C2 cDNA sequence (coding sequence)

SEQ ID No.18: Brassica napus CKX3-C2 amino acid sequence

SEQ ID No.19: Brassica napus CKX5-A1 genomic sequence

SEQ ID No.20: Brassica napus CKX5-A1 cDNA sequence (coding sequence)

SEQ ID No. 21: Brassica napus CKX5-A1 amino acid sequence

SEQ ID No.22: Brassica napus CKX5-C1 genomic sequence

SEQ ID No.23: Brassica napus CKX5-C1 cDNA sequence (coding sequence)

SEQ ID No.24: Brassica napus CKX5-C1 amino acid sequence

SEQ ID No.25: Brassica napus CKX3-A1 YIIN501 amino acid sequence

SEQ ID No.26: Brassica napus CKX3-A2 YIIN512 amino acid sequence

SEQ ID No.27: Brassica napus CKX3-C1 YIIN521 amino acid sequence

SEQ ID No.28: Brassica napus CKX3-C2 YIIN531 amino acid sequence

SEQ ID No.29: Brassica napus CKX5-A1 YIIN801 amino acid sequence

SEQ ID No.30: Brassica napus CKX5-A1 YIIN805 amino acid sequence

SEQ ID No.31: Brassica napus CKX5-C1 YIIN811 amino acid sequence

SEQ ID No. 32: KASP Primer BnCKX3-A1 WT allele

SEQ ID No. 33: KASP Primer BnCKX3-A1 YIIN501 allele

SEQ ID No. 34: KASP Primer BnCKX3-A2 WT allele

SEQ ID No. 35: KASP Primer BnCKX3-A2 YIIN512 allele

SEQ ID No. 36: KASP Primer BnCKX3-C1 WT allele

SEQ ID No. 37: KASP Primer BnCKX3-C1 YIIN521 allele

SEQ ID No. 38: KASP Primer BnCKX3-C2 WT allele

SEQ ID No. 39: KASP Primer BnCKX3-C2 YIIN531 allele

SEQ ID No. 40: KASP Primer BnCKX5-A1 WT allele

SEQ ID No. 41: KASP Primer BnCKX5-YIIN801 WT allele

SEQ ID No. 42: KASP Primer BnCKX5-A1 WT allele

SEQ ID No. 43: KASP Primer BnCKX5-A1 YIIN805 allele

SEQ ID No. 44: KASP Primer BnCKX5C1 WT allele

SEQ ID No. 45: KASP Primer BnCKX5-C1 YIIN811 allele

### EXAMPLES

Unless stated otherwise in the Examples, all recombinant DNA techniques are carried out according to standard protocols as described in Sambrook and Russell (2001) Molecular Cloning: A Laboratory Manual, Third Edition, Cold Spring Harbor Laboratory Press, NY, in Volumes 1 and 2 of Ausubel et al. (1994) Current Protocols in Molecular Biology, Current Protocols, USA and in Volumes I and II of Brown (1998) Molecular Biology LabFax, Second Edition, Academic Press (UK). Standard materials and methods for plant molecular work are described in Plant Molecular Biology Labfax (1993) by R. D. D. Croy, jointly published by BIOS Scientific Publications Ltd (UK) and Blackwell Scientific Publications, UK. Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1—Isolation of the DNA Sequences of the CKX3 and CKX5 Genes

The CKX3 and CKX5 nucleotide sequences from Brassica napus have been determined as follows.

Genomic DNA from Brassica napus was isolated using standard procedures. Fragments of the CKX3 and CKX5 genes were isolated through PCR on the B. napus genomic DNA using primers based on the A. thaliana CKX3 and CKX5 gene sequence as described. The PCR products were cloned and the sequence was determined.

Subsequently, CKX3 and CKX5 sequences from the PCR products were used as the query in a BLAST homology search of in-house sequence databases of a Brassica napus line. Four CKX3 genes were identified in B. napus, and two CKX5 genes. The genes and coding regions of the CKX3 and CKX5 sequences were determined using EST sequence information and comparison with the Arabidopsis CKX3 gene At5g56970 and CKX5 gene At1g75450 sequence information. The Brassica CKX3 and CKX5 sequences have five exons.

SEQ ID NOs: 7, 10, 13 and 16 are the genomic sequences of BnCKX3-A1, Bn CKX3-A2, Bn CKX3-C1 and Bn CKX3-C2, respectively of B. napus. SEQ ID NOs: 8, 11, 14 and 17 are the cDNA (coding) sequences of Bn CKX3-A1, Bn CKX3-A2, Bn CKX3-C1 and Bn CKX3-C2, respectively. Amino acid sequences of the proteins encoded by Bn CKX3-A1, Bn CKX3-A2, Bn CKX3-C1 and Bn CKX3-C2 are given in SEQ ID NOs: 9, 12, 15 and 18, respectively.

SEQ ID NOs: 19 and 22 are the genomic sequences of BnCKX5-A1 and Bn CKX5-C1, respectively of B. napus. SEQ ID NOs: 20 and 23 are the cDNA (coding) sequences of BnCKX5-A1 and Bn CKX5-C1, respectively. Amino acid sequences of the proteins encoded by BnCKX5-A1 and Bn CKX5-C1 are given in SEQ ID NOs: 21 and 24, respectively.

### Example 2—Generation and Isolation of Mutant CKX3 and CKX5 Alleles

Mutations in the CKX3 and CKX5 genes of Brassica napus identified in Example 1 were generated and identified as follows:
30,000 seeds from an elite spring oilseed rape breeding line (M0 seeds) were pre-imbibed for 2 h on wet filter paper in deionized or distilled water. Half of the seeds were exposed to 0.8% EMS and half to 1% EMS (Sigma: M0880) and incubated for 4 h.
The mutagenized seeds (M1 seeds) were rinsed three times and dried in a fume hood overnight. 30,000 M1 plants were grown in soil and selfed to generate M2 seeds. M2 seeds were harvested for each individual M1 plant.
Two times 4800 M2 plants, derived from different M1 plants, were grown and DNA samples were prepared from leaf samples of each individual M2 plant according to the CTAB method (Doyle and Doyle, 1987, Phytochemistry Bulletin 19:11-15).
The DNA samples were screened for the presence of point mutations in the CKX3 and CKX5 genes that cause the introduction of STOP codons or introduction of another amino acid in the protein-encoding regions of the CKX3 and CKX5 genes, by direct sequencing by standard sequencing techniques (LGC) and analyzing the sequences for the presence of the point mutations using the NovoSNP software (VIB Antwerp).
The mutant CKX3 and CKX5 genes alleles as depicted in Table 3 were thus identified.

### Example 3—Identification of a Brassica Plant Comprising Mutant Brassica CKX5 or CKX5 and CKX3 Alleles

Brassica plants comprising the mutations in the CKX5 and CKX3 genes identified in Example 2 were identified as follows:
For each mutant CKX3 or CKX5 gene identified in the DNA sample of an M2 plant, at least 50 M2 plants derived from the same M1 plant as the M2 plant comprising the CKX3 or CKX5 mutation, were grown and DNA samples were prepared from leaf samples of each individual M2 plant.
The DNA samples were screened for the presence of the identified point CKX3 or CKX5 mutation as described above in Example 4.
Heterozygous and homozygous (as determined based on the electropherograms) M2 plants comprising the same mutation were selfed and M3 seeds were harvested.

### Example 4—Analysis of Brassica Plants Comprising Mutant Brassica CKX5 and CKX3 alleles in Greenhouse Conditions

Brassica plants homozygous for mutations in all CKX5 and CKX3 genes were grown under greenhouse conditions until complete maturity. The summary results of flower counts on Brassica plants homozygous for mutations in all CKX5 and CKX3 genes in growth chamber conditions in absence of forcing pollination techniques (no bag selfs, no insect pollinators). The specific objective was to determine the absolute effect of the CKX5/CKX3 mutants on the number of flowers per plant and on the distribution of the effect across the branches.

The following Brassica plants were tested:

| Pedigree | Short Name |
|---|---|
| BC4S4 (YIIN501/YIIN512/YIIN521/YIIN531/ | CKX3/CKX5 6x (1) |
| YIIN801/YIIN811) | |
| (A1A1/A2A2/C1C1/C2C2/A1A1/C1C1) | |
| BC4S4 (YIIN501/YIIN512/YIIN521/YIIN531/ | CKX3/CKX5 WTS |
| YIIN801/YIIN811) (—/—/—/—/—) | (1) |
| BC4S4 (YIIN501/YIIN512/YIIN521/YIIN531/ | CKX3/CKX5 6x (2) |
| YIIN805/YIIN811) | |
| (A1A1/A2A2/C1C1/C2C2/A1A1/C1C1) | |
| BC4S4 (YIIN501/YIIN512/YIIN521/YIIN531/ | CKX3/CKX5 WTS |
| YIIN805/YIIN811) (—/—/—/—/—) | (2) |
| Original Brassica line not subjected to mutagenesis | WT |

The following variables were scored and measured in the trial:

| | | | | | | |
|---|---|---|---|---|---|---|
| Variable | Abbreviation | Stage | Scale or Unit | Scale 1 | Scale 5 | Scale 9 |

| | | | |
|---|---|---|---|
| Number of branches (incl. | NBR | flowering end | number |
| main) | | | |
| Number of flowers (total) | NFL ¹ | flowering end | number |
| Number of flowers on | NFLM ¹ | flowering end | number |
| main branch | | | |
| Number of flowers on side | NFLS ¹ | flowering end | number |
| branches (per branch, in | | | |
| order from top to bottom) | | | |
| Percent flowers on main | PFLM ¹ | flowering end | percent ² |
| branch | | | |
| Percent flowers on side | PFLS1 till | flowering end | percent ² |
| branches (per branch, in | PFLS7 ¹ | | |
| order from top to bottom) | | | |

¹ (based on) sum of open flowers, aborted flowers and pods
² calculated (based on NFL counts)

An overview of the overall estimates from an ANOVA analysis (with random block effect) for the variables is shown in Table 4, including significance testing (p<0.05) for the contrasts between the mutant lines and corresponding wild type segregants (*) and for the contrasts between wild type segregants (WTS) and the wild type (WT) check (**). Note that absence of a statistical difference does not imply equivalence.

TABLE 4
Overall estimates

| | NBR | NFL | NFLM | NFLS1 | NFLS2 | NFLS3 | NFLS4 | NFLS5 | NFLS6 | NFLS7 |
|---|---|---|---|---|---|---|---|---|---|---|
| GENOTYPES | nr | nr | nr | nr | nr | nr | nr | nr | nr | nr |
| CKX3/CKX5 6x (1) | 6.3* | 926.2* | 131.0* | 135.9 | 162.7* | 162.3* | 157.1* | 134.5* | 42.8 | 0.0 |
| CKX3/CKX5 WTS (1) | 5.6 | 616.1 | 101.7** | 128.1 | 107.3 | 121.6 | 77.7** | 57.0 | 21.2 | 1.7 |
| CKX3/CKX5 6x (2) | 5.4 | 838.5* | 137.8* | 161.7* | 163.9* | 164.6* | 141.4 | 69.2 | 0.0 | 0.0 |
| CKX3/CKX5 WTS (2) | 5.8 | 623.1 | 92.8 | 98.7 | 122.9 | 115.0 | 110.6 | 71.5 | 11.7 | 0.0 |
| WT | 5.8 | 584.5 | 90.7 | 105.8 | 101.4 | 103.9 | 115.5 | 62.0 | 5.4 | 0.0 |
| CV | 11.4 | 10.0 | 12.7 | 34.6 | 30.3 | 27.8 | 44.2 | 78.7 | 257.1 | >999 |

| | PFLM | PFLS1 | PFLS2 | PFLS3 | PFLS4 | PFLS5 | PFLS6 | PFLS7 |
|---|---|---|---|---|---|---|---|---|
| GENOTYPES | % | % | % | % | % | % | % | % |
| CKX3/CKX5 6x (1) | 14.2* | 14.7* | 17.5 | 17.6 | 16.9 | 14.5* | 4.7 | 0.0 |
| CKX3/CKX5 WTS (1) | 16.6 | 21.0 | 17.5 | 19.9 | 12.8** | 8.9 | 3.0 | 0.2 |
| CKX3/CKX5 6x (2) | 16.5* | 19.4 | 19.6 | 19.6 | 16.6 | 8.1 | 0.0 | 0.0 |
| CKX3/CKX5 WTS (2) | 15.1 | 15.9 | 19.8 | 18.4 | 18.0 | 11.1 | 1.5 | 0.0 |
| WT | 15.6 | 18.2 | 17.3 | 17.9 | 19.6 | 10.4 | 0.9 | 0.0 |
| CV | 15.7 | 33.4 | 29.5 | 27.5 | 41.6 | 78.2 | 258.4 | >999 |

*mutant significantly different from WTS
**WTS significantly different from the WT check

Both lines of Brassica plants homozygous for mutations in all CKX5 and CKX3 genes demonstrate a highly significant increase in number of flowers. The increase in number of flowers is equally divided across the flower branches.

### Example 5—Analysis of Brassica Plants Comprising Mutant Brassica CKX5 and CKX3 Alleles Under Field Conditions

Brassica plants homozygous for mutations in all CKX5, all CKX3 genes and all CKX5 and CKX3 genes were grown under field conditions in various locations in Europe and North America.

The following Brassica plants were tested:

| Pedigree | Short Name |
|---|---|
| BC4S4 (YIIN501/YIIN512/YIIN521/YIIN531/ | CKX3/CKX5 6x (1) |
| YIIN801/YIIN811) | |
| (A1A1/A2A2/C1C1/C2C2/A1A1/C1C1) | |
| BC4S4 (YIIN501/YIIN512/YIIN521/YIIN531/ | CKX3/CKX5 WTS (1) |
| YIIN801/YIIN811) (—/—/—/—/—/—) | |
| BC4S4 (YIIN501/YIIN512/YIIN521/YIIN531/ | CKX3/CKX5 6x (2) |
| YIIN805/YIIN811) | |
| (A1A1/A2A2/C1C1/C2C2/A1A1/C1C1) | |
| BC4S4 (YIIN501/YIIN512/YIIN521/YIIN531/ | CKX3/CKX5 WTS (2) |
| YIIN805/YIIN811) (—/—/—/—/—/—) | |
| BC4S3 (YIIN801/YIIN811) (A1A1/C1C1) | CKX5 2x (1) |
| BC4S3 (YIIN801/YIIN811) (—/—) | CKX5 WTS (1) |
| BC4S3 (YIIN805/YIIN811) (A1A1/C1C1) | CKX5 2x (2) |
| BC4S3 (YIIN805/YIIN811) (—/—) | CKX5 WTS (2) |
| BC5S3 (YIIN501/YIIN512/YIIN521/YIIN531) | CKX3 4x |
| (A1A1/A2A2/C1C1/C2C2) | |
| BC5S3 (YIIN501/YIIN512/YIIN521/YIIN531) | CKX3 WTS |
| (—/—/—/—) | |
| Original Brassica line not subjected to | WT |
| mutagenesis | |

The following variables were scored and measured in the trial:

| Variable | Abbreviation | Stage | Scale or Unit | Scale 1 | Scale 5 | Scale 9 |
|---|---|---|---|---|---|---|
| Number of flowers on | NFLM | flowering | number/plant | | | |
| main branch | | | | | | |
| Flowering-End | EOF | 90% flowering | days after seeding | | | |
| | | end | | | | |
| Plant Height | HICM | flowering end | cm | | | |
| Number of pods | NPOD | maturity | number/plant | | | |
| (total) | | | | | | |
| Percent pods on main | PPODM | maturity | %/plant | | | |
| branch | | | | | | |
| Maturity | DTM | maturity | days after seeding | | | |
| Lodging Resistance at | LOM | maturity | (1-9) | 0 degrees | 45 degrees | 90 degrees |
| Maturity | | | | (flat) | | (upright) |
| Seed yield per plot at | YLD | seed | grams/plot | | | |
| 8% moisture | | | | | | |
| Seed yield per plant at | YLDPL ¹ | seed | grams/plant | | | |
| 8% moisture | | | | | | |
| Seeds per pod on | SPODM ² | seed | number/pod | | | |
| main branch | | | | | | |
| Thousand Seed Weight | TSW | seed | grams/1000 seeds | | | |
| NIR analysis | GLUN | seed | μmoles/gram | | | |
| | OILN | | % of whole seed | | | |
| | PRON | | % of whole seed | | | |

${}^{1} YLDPL = \frac{YLD}{PLDE}$ ² SPODM: average of 4 oldest pods on the main branch

An overview of the overall estimates from an ANOVA analysis (with random block effect) for the variables is shown in Table 5 for European field trials and Table 6 for North American field trials, including significance testing (p<0.05) for the contrasts between the mutant lines and corresponding wild type segregants (*) and for the contrasts between wild type segregants (WTS) and the wild type (WT) check (**). Note that absence of a statistical difference does not imply equivalence.

TABLE 5
Overall estimates (Europe)

| | LOM | YLD | YLDPL | TSW | OILN | PRON | GLUN | NPOD | PPODM | SPODM |
|---|---|---|---|---|---|---|---|---|---|---|
| GENOTYPES | (1-9) | gram | gram | gram | % | % | μmol/g | pods | % | seeds |
| CKX3/CKX5 6x (1) | 9.0 | 2251 | 3.44 | 3.81* | 47.8 | 22.3 | 12.5* | 77.5 | 57.6* | 42.6* |
| CKX3/CKX5 WTS (1) | 9.0 | 2281 | 3.65 | 3.42 | 48.1 | 22.1 | 10.8 | 83.4** | 43.1 | 37.6 |
| CKX3/CKX5 6x (2) | 9.0 | 2220 | 3.50 | 4.02* | 47.0* | 23.0 | 13.0* | 80.5 | 55.6* | 42.6* |
| CKX3/CKX5 WTS (2) | 9.0 | 2348 | 3.56 | 3.37 | 48.2 | 22.3 | 11.2 | 77.7 | 45.9 | 35.1 |
| CKX5 2x (1) | 9.0 | 2312 | 3.71 | 3.58* | 47.6 | 22.3 | 11.4* | 76.3 | 48.5* | 39.4* |
| CKX5 WTS (1) | 9.0 | 2339 | 3.74 | 3.35 | 48.3 | 21.8 | 10.2 | 89.4** | 42.0 | 36.6 |
| CKX5 2x (2) | 9.0 | 2337 | 3.65 | 3.67* | 47.5 | 22.5 | 11.2 | 79.4 | 47.6 | 40.9* |
| CKX5 WTS (2) | 9.0 | 2362 | 3.96** | 3.40 | 47.9 | 22.7** | 11.1 | 83.5** | 44.0 | 33.1** |
| CKX3 4x | 9.0 | 2485 | 3.51 | 3.55* | 48.3 | 22.1 | 11.5 | 77.2 | 47.7 | 36.2 |
| CKX3 WTS | 9.0 | 2467 | 3.58 | 3.42 | 48.0 | 22.2 | 11.5 | 74.4 | 47.4 | 35.4 |
| 98-55-013 | 9.0 | 2338 | 3.42 | 3.37 | 48.6 | 21.7 | 10.6 | 70.9 | 46.9 | 36.0 |
| CV | 0.0 | 10.1 | 18.1 | 2.9 | 2.4 | 4.3 | 11.4 | 41.5 | 36.1 | 10.2 |

TABLE 6
Overall estimates (North America)

| | EST1 | PLDE | VIG1 | DTF | EOF | HICM | DTM | LOM | YLD | YLDPL | TSW | OILN | PRON | GLUN | NFLM |
|---|---|---|---|---|---|---|---|---|---|---|---|---|---|---|---|
| GENOTYPES | (1-9) | plants | (1-9) | days | days | cm | days | (1-9) | gram | gram | gram | % | % | μmol/g | flowers |

| | |
|---|---|
| CKX3/CKX5 6x (1) | 63.9* |
| CKX3/CKX5 WTS (1) | 49.3 |
| CKX3/CKX5 6x (2) | 60.5* |
| CKX3/CKX5 WTS (2) | 52.0 |
| CKX5 2x (1) | 51.9* |
| CKX5 WTS (1) | 49.0 |
| CKX5 2x (2) | 53.6* |
| CKX5 WTS (2) | 50.5 |
| CKX3 4x | 54.3* |
| CKX3 WTS | 50.6 |
| WT | 50.1 |
| CV | 12.9 |

In general the following conclusions can be drawn:
a. more flowers on main branch (side branches not counted for)
b. more pods on the main branch
c. more seeds per pod on the main branch (side branches not measured)
d. higher TSW (of seed bulks main+side branches)
e. only a limited effect on seed yield
f. no demonstrable effect on seed yield per plant which may be due to variability in plant density between plots and plant distance within each plot

More specifically:
a. Mutant ckx3/ckx5 6x (1) demonstrates the highest increase on number of flowers (29%) and number of pods (33%) on the main branch, but only 11% increase on TSW and 13% increase on number of seeds per pod on the main branch. There is no effect on seed yield.
b. Both CKX5 mutants show intermediate effect on TSW, number of flowers and pods on the main branch, without an effect on seed yield. Unclear is the highest ranking of YIIN805/YIIN811 for number of seeds per pod on the main branch without direct correlation with the results of the parameters for the other mutants.

### Example 6—Detection and/or Transfer of Mutant CKX5 and CKX3 Genes into (Elite) Brassica Lines

To select for plants comprising a point mutation in a CKX5 or CKX3 allele, direct sequencing by standard sequencing techniques known in the art, such as those described in Example 2, can be used. Alternatively, PCR based assays can be developed to discriminate plants comprising a specific point mutation in a CKX5 or CKX3 allele from plants not comprising that specific point mutation. The following KASP assays were developed to detect the presence or absence and the zygosity status of the mutant alleles identified in Example 2 (see Table 3):
Template DNA:

Genomic DNA isolated from leaf material of homozygous or heterozygous mutant Brassica plants (comprising a mutant CKX5 or CKX3 allele, called hereinafter “CKXx-Xx-YIINxxx”).

Wild type DNA control: Genomic DNA isolated from leaf material of wild type Brassica plants (comprising the wild type equivalent of the mutant CKX5 or CKX3 allele, called hereinafter “WT”).

Positive DNA control: Genomic DNA isolated from leaf material of homozygous mutant Brassica plants known to comprise CKXx-Xx-YIINxxx.
Primers and probes for the mutant and corresponding wild type target CKX5 or CKX3 gene are indicated in Table 7.

TABLE 7
Primers and probes for detection of wild type and mutant CKX5 or
CKX3 alleles

| target | mutant | Primer_WT allele | Primer_MUT allele |
|---|---|---|---|
| CKX3- | YIIN | GAAGGTGACCAAGTTCATGCTGTATATGGATTTCTT | GAAGGTCGGAGTCAACGGATTCGTATATGGATTTCTTA |
| A1 | 501 | AAACCGGGTTC (SEQ ID No. 32) | AACCGGGTTT (SEQ ID No. 33) |
| CKX3- | YIIN | GAAGGTGACCAAGTTCATGCTGGCTTAATCTCTTTG | GAAGGTCGGAGTCAACGGATTATGGCTTAATCTCTTTG |
| A2 | 512 | TACCAAAATCTC (SEQ ID No. 34) | TACCAAAATCTT (SEQ ID No. 35) |
| CKX3- | YIIN | GAAGGTGACCAAGTTCATGCTGGTGGATAGTAAGTG | GAAGGTCGGAGTCAACGGATTCGGTGGATAGTAAGTG |
| C1 | 521 | GACCGC (SEQ ID No. 36) | GACCGT (SEQ ID No. 37) |
| CKX3- | YIIN | GAAGGTGACCAAGTTCATGCTGGACCTCGTTGACTG | GAAGGTCGGAGTCAACGGATTCGGACCTCGTTGACTG |
| C2 | 531 | AGTGTTG (SEQ ID No. 38) | AGTGTTA (SEQ ID No. 39) |
| CKX5- | YIIN | GAAGGTGACCAAGTTCATGCTCAACGGAAAGATAC | GAAGGTCGGAGTCAACGGATTCCAACGGAAAGATACA |
| A1 | 801 | AAGTAATCAGTC (SEQ ID No. 40) | AGTAATCAGTT (SEQ ID No. 41) |
| CKX5- | YIIN | GAAGGTGACCAAGTTCATGCTCATCAACCCATAACT | GAAGGTCGGAGTCAACGGATTACATCAACCCATAACT |
| A1 | 805 | CTCCACCC (SEQ ID No. 42) | CTCCACCT (SEQ ID No. 43) |
| CKX5- | YIIN | GAAGGTGACCAAGTTCATGCTCAACGGAAAGATAC | GAAGGTCGGAGTCAACGGATTCAACGGAAAGATACAG |
| C1 | 811 | AGGTAATCAGTC (SEQ ID No. 44) | GTAATCAGTT (SEQ ID No. 45) |

### Example 7: Further Analysis of Brassica Plants Comprising Mutant Brassica CKX5 and CKX3 alleles in Greenhouse Conditions

Brassica plants homozygous for mutations in all CKX5 and CKX3 genes were grown and phenotyped under greenhouse conditions. It should be noted that the plants were grown in larger pots than used for the greenhouse trial in Example 4. It is expected that growing denser in smaller pots reflects field conditions more accurately.

The following Brassica plants were tested:

| Pedigree | Short Names |
|---|---|
| BC4S4 (YIIN501/YIIN512/YIIN521/YIIN531/ | CKX3/CKX5 6x (1) |
| YIIN801/YIIN811) | Mutant 1 |
| (A1A1/A2A2/C1C1/C2C2/A1A1/C1C1) | |
| BC4S4 (YIIN501/YIIN512/YIIN521/YIIN531/ | CKX3/CKX5 WTS (1) |
| YIIN801/YIIN811) (—/—/—/—/—) | |
| wild type segregant | |
| BC4S4 (YIIN501/YIIN512/YIIN521/YIIN531/ | CKX3/CKX5 6x (2) |
| YIIN805/YIIN811) | |
| (A1A1/A2A2/C1C1/C2C2/A1A1/C1C1) | Mutant 2 |
| BC4S4 (YIIN501/YIIN512/YIIN521/YIIN531/ | CKX3/CKX5 WTS (2) |
| YIIN805/YIIN811) (—/—/—/—/—) | |
| wild type segregant | |
| Original Brassica line not subjected to | WT |
| mutagenesis | |

The following variables were scored and measured in the trial:

| Variable | Abbreviation | Stage | Unit |
|---|---|---|---|
| Number of branches | NBR | maturity | nr |
| Number of flowers on main branch | NFLM¹ | maturity | nr |
| Number of flowers on side branches | NFLS¹⁺² | maturity | nr |
| Number of pods on main branch | NPODM³ | maturity | nr |
| Number of pods on side branches | NPODS²⁺³ | maturity | nr |
| Seed Yield (weight) main branch | YLDWM | seed | g |
| | | | (accuracy |
| | | | mg) |
| Seed Yield (weight) side branches | YLDWS² | seed | g |
| | | | (accuracy |
| | | | mg) |
| Seed Yield (seed number) main | YLDSM | seed | nr |
| branch | | | |
| Seed Yield (seed number) side | YLDSS² | seed | nr |
| branches | | | |

¹sum of pods and aborted flower buds/pods
²for each side branch separately (number the branches from top to bottom, S1 till Sx with x = NBR − 1)
³all pods with thickening

The following variables were calculated:

| Variable | Abbreviation | Unit | Formula |
|---|---|---|---|
| Percent flowers main branch on total | PFLM ¹ | % | $PFLM = \frac{NFLM}{NFL} * 100$ |
| Percent flowers side branches on total | PFLS ¹⁺² | % | $PFLS = \frac{NFLS}{NFL} * 100$ |
| Percent aborted flowers/pods (total) | PABR | % | $PABR = \frac{NFL - NPOD}{NFL} * 100$ |
| Percent aborted flowers/pods on main branch | PABRM | % | $PABRM = \frac{NFLM - NPODM}{NFLM} * 100$ |
| Percent aborted flowers/pods on side branches | PABRS ² | % | $PABRS = \frac{NFLS - NPODS}{NFLS} * 100$ |
| Number of pods (total) | NPOD ³ | nr | NPOD = NPODM + NPODS |
| Percent pods main branch on total | PPODM ³ | % | $PPODM = \frac{NPODM}{NPOD} * 100$ |
| Percent pods side branches on total | PPODS ²⁺³ | % | $PPODS = \frac{NPODS}{NPOD} * 100$ |
| Seed Yield (total weight) | YLDW | g | YLDW = YLDWM + YLDWS |
| | | (accuracy mg) | |
| Percent seed yield (weight) main branch on total | PYLDWM | % | $PYLDWM = \frac{YLDWM}{YLDW} * 100$ |
| Percent seed yield (weight) side branches on total | PYLDWS ² | % | $PYLDWS = \frac{YLDWS}{YLDW} * 100$ |
| Seed Yield (total seed number) | YLDS | nr | YLDS = YLDSM + YLDSS |
| Percent seed yield (seed number) main branch on total | PYLDSM | % | $PYLDSM = \frac{YLDSM}{YLDS} * 100$ |
| Percent seed yield (seed number) side branches on total | PYLDSS ² | % | $PYLDSS = \frac{YLDSS}{YLDS} * 100$ |
| Number of seeds per pod (average per plant) | SPOD | nr | $SPOD = \frac{YLDS}{NPOD}$ |
| Number of seeds per pod on main branch | SPODM | nr | $SPODM = \frac{YLDSM}{NPODM}$ |
| Number of seeds per pod on side branches | SPODS ² | nr | $SPODS = \frac{YLDSS}{NPODS}$ |
| Seed Weight (average per plant) | SW | mg (accuracy μg) | $SW = \frac{YLDW}{YLDS} * 1000$ |
| Seed Weight on main branch | SWM | mg (accuracy μg) | $SWM = \frac{YLDWM}{YLDSM} * 1000$ |
| Seed Weight on side branches | SWS ² | mg (accuracy μg) | $SWS = \frac{YLDWS}{YLDSS} * 1000$ |

¹ sum of pods and aborted flower buds/pods
² for each side branch separately (number the branches from top to bottom, S1 till Sx with x = NBR-1)
³ all pods with thickening

An overview of the overall estimates from an ANOVA analysis for the variables is shown in Table 8, including significance testing (p<0.05) for the contrasts between the mutant lines and corresponding wild type segregants (*) and for the contrasts between wild type segregants and the wild type check (**). Note that absence of a statistical difference does not imply equivalence.

CKX mutants show an increased effect on number of pods (NPOD) on main branch and the two first side branches resulting in a higher total number of pods. The effect becomes less for lower branches, even resulting in a negative effect at the height of the middle side branch, but reveals again at the lowest branches without significant effect on the total number of pods due to the low amount of developed pods at those lowest branches. Looking at the effect on the contribution of each branch to the total number of pods (PPOD) then the main branch becomes even more dominant than it already is in the wild types.

The increased effect on number of pods is strengthened by a reduction in flower and pod abortions on the main and first 4 side branches.

The increased dominant effect of the main branch leads to higher seed yield weights on that main branch, but also to yield weight decreases on all side branches, resulting in a total seed yield weight decrease. This can explain why the negative effect on seed yield in the field trials was not that strong, because plants are growing at higher plant density with much less side branches.

The neutral effect on the number of seeds on the main branch indicates that the seeds are bigger, which is confirmed by the seed weight results. The increased seed weight (size) can be observed for all branches.

Different embodiments of the invention are summarized in the following paragraphs:
1. A Brassica plant comprising at least one CKX5 gene, comprising at least one mutant CKX5 allele in its genome.
2. The plant according to paragraph 1, wherein said mutant CKX5 allele is a mutant allele of a CKX5 gene comprising a nucleic acid sequence selected from the group consisting of:

(a) a nucleotide sequence which comprises at least 90% sequence identity to SEQ ID NO: 19 or SEQ ID NO: 23;

(b) a nucleotide sequence comprising a coding sequence which comprises at least 90% sequence identity to SEQ ID NO: 20 or SEQ ID NO: 23; and

(c) a nucleotide sequence encoding an amino acid sequence which comprises at least 90% sequence identity to SEQ ID NO: 21 or SEQ ID NO: 24.
3. The plant according to paragraph 1 or 2, which is a Brassica plant comprising two CKX5 genes, said Brassica plant selected from the group consisting of Brassica napus, Brassica juncea and Brassica carinata.
4. The plant according to any one of paragraphs 1 to 3, comprising at least two mutant CKX5, or at least three mutant CKX5 alleles, or at least four mutant CKX5 alleles.
5. The plant according to any one of paragraphs 1 to 4, wherein said mutant CKX5 allele is selected from the group consisting of:

(a) a mutant CKX5 allele comprising a G to A substitution at a position corresponding to position 465 of SEQ ID NO: 19 or position 465 of SEQ ID No. 20;

(b) a mutant CKX5 allele comprising a G to A substitution at a position corresponding to position 399 of SEQ ID NO: 19 or position 399 of SEQ ID No. 20; and

(c) a mutant CKX5 allele comprising a G to A substitution at a position corresponding to position 465 of SEQ ID NO: 22 or position 399 of SEQ ID No. 23.
6. The plant according to any one of paragraphs 1 to 5, which is homozygous for the mutant CKX5 allele.
7. The plant according to any one of paragraphs 1 to 6 further comprising at least two CKX3 genes, further comprising at least two mutant CKX3 alleles in its genome.
8. The plant according to paragraph 7, wherein said mutant CKX3 allele is a mutant allele of a CKX3 gene comprising a nucleic acid sequence selected from the group consisting of:

(a) a nucleotide sequence which comprises at least 90% sequence identity to SEQ ID NO: 7, SEQ ID NO: 10; SEQ ID NO: 13 or SEQ ID NO: 16;

(b) a nucleotide sequence comprising a coding sequence which comprises at least 90% sequence identity to SEQ ID NO: 8, SEQ ID NO: 11; SEQ ID NO: 14 or SEQ ID NO: 17; and

(c) a nucleotide sequence encoding an amino acid sequence which comprises at least 90% sequence identity to SEQ ID NO: 9, SEQ ID NO: 12; SEQ ID NO: 15 or SEQ ID NO: 18.

9. The plant according to paragraph 7 or 8, which is a Brassica plant comprising four CKX3 genes, said Brassica plant selected from the group consisting of Brassica napus, Brassica juncea and Brassica carinata.

10. The plant according to any one of paragraphs 7 to 9, comprising at least two mutant CKX3 alleles, or at least three mutant CKX3 alleles, or at least four mutant CKX3 alleles, or at least five mutant CKX3 alleles, or at least six mutant CKX3 alleles, or at least seven mutant CKX3 alleles, or at least eight mutant CKX3 alleles.
11. The plant according to any one of paragraphs 7 to 10, wherein said mutant CKX3 allele is selected from the group consisting of:

a mutant CKX3 allele comprising a C to T substitution at a position corresponding to position 2244 of SEQ ID NO: 7 or position 1093 of SEQ ID No. 8;

a mutant CKX3 allele comprising a C to T substitution at a position corresponding to position 2482 of SEQ ID NO: 10 or position 1168 of SEQ ID No. 11;

a mutant CKX3 allele comprising a G to A substitution at a position corresponding to position 1893 of SEQ ID NO: 13 or position 876 of SEQ ID No. 14;

a mutant CKX3 allele comprising a C to T substitution at a position corresponding to position 2171 of SEQ ID NO: 16 or position 982 of SEQ ID No. 17.
12. The plant according to any one of paragraphs 1 to 5, which is homozygous for the mutant CKX3 allele.
13. A Brassica plant comprising at least two CKX5 genes, wherein expression of at least one CKX5 gene is reduced.
14. The plant according to any one of paragraphs 1 to 13, which has increased flower number per plant.
15. The plant according to any one of paragraphs 1 to 13 which has an increased Thousand Seed Weight.
16. A plant cell, pod, seed, or progeny of the plant of any one of paragraphs 1 to 15.
17. A mutant allele of a Brassica CKX3 or CKX5 gene, wherein the CKX5 gene is selected from the group consisting of:

(a) a nucleotide sequence which comprises at least 90% sequence identity to SEQ ID NO: 19 or SEQ ID NO: 23;

(b) a nucleotide sequence comprising a coding sequence which comprises at least 90% sequence identity to SEQ ID NO: 20 or SEQ ID NO: 23; and

(c) a nucleotide sequence encoding an amino acid sequence which comprises at least 90% sequence identity to SEQ ID NO: 21, or SEQ ID NO: 24; and wherein the CKX3 gene is selected from the group consisting of

(d) a nucleotide sequence which comprises at least 90% sequence identity to SEQ ID NO: 7, SEQ

ID NO: 10; SEQ ID NO: 13 or SEQ ID NO: 16;

(e) a nucleotide sequence comprising a coding sequence which comprises at least 90% sequence identity to SEQ ID NO: 8, SEQ ID NO: 11; SEQ ID NO: 14 or SEQ ID NO: 17; and

(f) a nucleotide sequence encoding an amino acid sequence which comprises at least 90% sequence identity to SEQ ID NO: 9, SEQ ID NO: 12; SEQ ID NO: 15 or SEQ ID NO: 18.
18. The mutant allele according to paragraph 17, selected from the group consisting of:

a. a mutant CKX5 allele comprising a G to A substitution at a position corresponding to position 465 of SEQ ID NO: 19 or position 465 of SEQ ID No. 20;

b. a mutant CKX5 allele comprising a G to A substitution at a position corresponding to position 399 of SEQ ID NO: 19 or position 399 of SEQ ID No. 20; and

c. a mutant CKX5 allele comprising a G to A substitution at a position corresponding to position 465 of SEQ ID NO: 22 or position 399 of SEQ ID No. 23;

d. a mutant CKX3 allele comprising a C to T substitution at a position corresponding to position 2244 of SEQ ID NO: 7 or position 1093 of SEQ ID No. 8;

e. a mutant CKX3 allele comprising a C to T substitution at a position corresponding to position 2482 of SEQ ID NO: 10 or position 1168 of SEQ ID No. 11;

f. a mutant CKX3 allele comprising a G to A substitution at a position corresponding to position 1893 of SEQ ID NO: 13 or position 876 of SEQ ID No. 14;

g. a mutant CKX3 allele comprising a C to T substitution at a position corresponding to position 2171 of SEQ ID NO: 16 or position 982 of SEQ ID No. 17.
19. A chimeric gene comprising the following operably linked DNA fragments:

(a) a plant-expressible promoter;

(b) a DNA region, which when transcribed yields an RNA or protein molecule inhibitory to the expression or activity of one or more CKX5 or CKX5 and CKX3 genes or proteins; and, optionally,

(c) a 3′ end region involved in transcription termination and polyadenylation.
20. A method for identifying a mutant CKX5 or CKX3 allele according to paragraph 17 or 18 in a biological sample, which comprises determining the presence of a mutant CKX5 or CKX3 specific region in a nucleic acid present in said biological sample.
21. A method for determining the zygosity status of a mutant CKX3 or CKX5 allele according to paragraph 17 or 18 in a Brassica plant, plant material or seed, which comprises determining the presence of a mutant and/or a corresponding wild type CKX3 or CKX5 specific region in the genomic DNA of said plant, plant material or seed.
22. A kit for identifying a mutant CKX3 or CKX5 allele according to paragraph 17 or 18, in a biological sample, comprising a set of at least two primers, said set being selected from the group consisting of:

(a) a set of primers, wherein one of said primers specifically recognizes the 5′ or 3′ flanking region of the mutant allele and the other of said primers specifically recognizes the mutation region of the mutant CKX3 or CKX5 allele, and

(b) a set of primers, wherein one of said primers specifically recognizes the 5′ or 3′ flanking region of the mutant CKX3 or CKX5 allele and the other of said primers specifically recognizes the joining region between the 3′ or 5′ flanking region and the mutation region of the mutant CKX3 or CKX5 allele, respectively;

or said kit comprising a set of at least one probe, said probe being selected from the group consisting of:

(a) a probe specifically recognizing the mutation region of the mutant CKX3 or CKX5 allele, and

(b) a probe specifically recognizing the joining region between the 3′ or 5′ flanking region between the mutation region of the mutant CKX3 or CKX5 allele.
23. A method for transferring at least one selected mutant CKX3 or CKX5 allele according to paragraph 17 or 18, from one plant to another plant comprising the steps of:

(a) identifying a first plant comprising at least one selected mutant CKX3 or CKX5 allele using the method according to paragraph 22,

(b) crossing the first plant with a second plant not comprising the at least one selected mutant CKX3 or CKX5 allele and collecting F1 hybrid seeds from said cross,

(c) optionally, identifying F1 plants comprising the at least one selected mutant CKX3 or CKX5 allele using the method according to paragraph 22,

(d) backcrossing the F1 plants comprising the at least one selected mutant CKX3 or CKX5 allele with the second plant not comprising the at least one selected mutant CKX3 or CKX5 allele for at least one generation (x) and collecting BCx seeds from said crosses, and

(e) identifying in every generation BCx plants comprising the at least one selected mutant CKX3 or CKX5 allele using the method according to the method of paragraph 22.
24. A method to increase flower number per plant, comprising

a. introducing at least one mutant CKX5 allele or at least one mutant CKX5 allele and one mutant CKX3 allele into a Brassica plant; or

b. introducing the chimeric gene of paragraph 19 into a Brassica plant.
25. A method to increase Thousand Seed Weight of seed of a Brassica plant, comprising

a. introducing at least one mutant CKX5 allele or at least one mutant CKX5 allele and one mutant CKX3 allele into a Brassica plant; or

b. introducing the chimeric gene of paragraph 19 into a Brassica plant.
26. A method to increase pod number per plant, comprising

a. introducing at least one mutant CKX5 allele or at least one mutant CKX5 allele and one mutant CKX3 allele into a Brassica plant; or

b. introducing the chimeric gene of paragraph 19 into a Brassica plant.
27. A method for production of seeds, said method comprising sowing the seeds according to paragraph 16, growing plants from said seeds, and harvesting seeds from said plants.
28. A Brassica plant selected from the group consisting of:

a Brassica plant comprising a mutant CKX5 allele comprising a G to A substitution at a position corresponding to position 465 of SEQ ID NO: 19 or position 465 of SEQ ID No. 20, reference seeds comprising said allele having been deposited at the NCIMB Limited on 5 Oct. 2015, under accession number NCIMB 42464;

a Brassica plant comprising a mutant CKX5 allele comprising a G to A substitution at a position corresponding to position 399 of SEQ ID NO: 19 or position 399 of SEQ ID No. 20 reference seeds comprising said allele having been deposited at the NCIMB Limited on 5 Oct. 2015, under accession number NCIMB 42465;

a Brassica plant comprising a mutant CKX5 allele comprising a G to A substitution at a position corresponding to position 465 of SEQ ID NO: 22 or position 399 of SEQ ID No. 23, reference seeds comprising said allele having been deposited at the NCIMB Limited on 5 Oct. 2015, under accession number NCIMB 42464;

a Brassica plant comprising a mutant CKX3 allele comprising a C to T substitution at a position corresponding to position 2244 of SEQ ID NO: 7 or position 1093 of SEQ ID No. 8, reference seeds comprising said allele having been deposited at the NCIMB Limited on 5 Oct. 2015, under accession number NCIMB 42464;

a Brassica plant comprising a mutant CKX3 allele comprising a C to T substitution at a position corresponding to position 2482 of SEQ ID NO: 10 or position 1168 of SEQ ID No. 11, reference seeds comprising said allele having been deposited at the NCIMB Limited on 5 Oct. 2015, under accession number NCIMB 42464;

a Brassica plant comprising a mutant CKX3 allele comprising a G to A substitution at a position corresponding to position 1893 of SEQ ID NO: 13 or position 876 of SEQ ID No. 14, reference seeds comprising said allele having been deposited at the NCIMB Limited on 5 Oct. 2015, under accession number NCIMB 42464;

a Brassica plant comprising a mutant CKX3 allele comprising a C to T substitution at a position corresponding to position 2171 of SEQ ID NO: 16 or position 982 of SEQ ID No. 17, reference seeds comprising said allele having been deposited at the NCIMB Limited on 5 Oct. 2015, under accession number NCIMB 42464;
29. Use of the mutant CKX5 allele or mutant CKX5 and mutant CKX3 alleles according to paragraph 17 or 18 or the chimeric gene according to paragraph 19 to increase flower number per plant, pod number per plant or increase TSW in Brassica plants.
30. Use of the Brassica plants according to any one of paragraphs 1 to 15, or of the seeds according to paragraph 27, to produce oilseed rape oil or an oilseed rape seed cake.
31. A method for producing food, feed, or an industrial product comprising

a. obtaining the plant or a part thereof, of any one of paragraphs 1 to 15 or the seeds of paragraph 27, and

b. preparing the food, feed or industrial product from the plant or part thereof.
32. The method of paragraph 31, wherein

a. the food or feed is oil, meal, grain, starch, flour or protein; or

b. the industrial product is biofuel, industrial chemicals, a pharmaceutical or a nutraceutical.

1. A Brassica plant or plant part thereof comprising at least one CKX5 gene, comprising at least one mutant CKX5 allele in its genome, wherein said mutant CKX5 allele is a mutant allele of a CKX5 gene comprising:
(a) a nucleotide sequence which comprises at least 90% sequence identity to SEQ ID NO: 19 or SEQ ID NO: 23;
(b) a nucleotide sequence comprising a coding sequence which comprises at least 90% sequence identity to SEQ ID NO: 20 or SEQ ID NO: 23; or
(c) a nucleotide sequence encoding an amino acid sequence which comprises at least 90% sequence identity to SEQ ID NO: 21 or SEQ ID NO: 24.

2. The plant according to claim 1, which is a Brassica plant comprising two CKX5 genes, said Brassica plant is Brassica napus, Brassica juncea and or Brassica carinata.

3. The plant according to claim 1, wherein said mutant CKX5 allele is:
(a) a mutant CKX5 allele comprising a G to A substitution at a position corresponding to position 465 of SEQ ID NO: 19 or position 465 of SEQ ID No. 20;
(b) a mutant CKX5 allele comprising a G to A substitution at a position corresponding to position 399 of SEQ ID NO: 19 or position 399 of SEQ ID No. 20; or
(c) a mutant CKX5 allele comprising a G to A substitution at a position corresponding to position 465 of SEQ ID NO: 22 or position 399 of SEQ ID No. 23.

4. The plant according to claim 1 further comprising at least two CKX3 genes, further comprising at least two mutant CKX3 alleles in its genome, wherein said mutant CKX3 allele is a mutant allele of a CKX3 gene comprising:
(a) a nucleotide sequence which comprises at least 90% sequence identity to SEQ ID NO: 7, SEQ ID NO: 10; SEQ ID NO: 13 or SEQ ID NO: 16;
(b) a nucleotide sequence comprising a coding sequence which comprises at least 90% sequence identity to SEQ ID NO: 8, SEQ ID NO: 11; SEQ ID NO: 14 or SEQ ID NO: 17; or
(c) a nucleotide sequence encoding an amino acid sequence which comprises at least 90% sequence identity to SEQ ID NO: 9, SEQ ID NO: 12; SEQ ID NO: 15 or SEQ ID NO: 18.

5. The plant according to claim 3, which is a Brassica plant comprising four CKX3 genes, said Brassica plant is Brassica napus, Brassica juncea or Brassica carinata.

6. The plant according to claim 5, wherein said mutant CKX3 allele is:
a mutant CKX3 allele comprising a C to T substitution at a position corresponding to position 22 of SEQ ID NO: 7 or position 1093 of SEQ ID No. 8;
a mutant CKX3 allele comprising a C to T substitution at a position corresponding to position 2482 of SEQ ID NO: 10 or position 1168 of SEQ ID No. 11;
a mutant CKX3 allele comprising a G to A substitution at a position corresponding to position 1893 of SEQ ID NO: 13 or position 876 of SEQ ID No. 14; or
a mutant CKX3 allele comprising a C to T substitution at a position corresponding to position 2171 of SEQ ID NO: 16 or position 982 of SEQ ID No. 17.

7. The plant according to claim 1, which has increased flower number per plant or which has increased number of pods per plant, or increased number of pods on the main branch, or has an increased Thousand Seed Weight compared to a Brassica plant without the at least one mutant CKX5 allele.

8. A plant cell, pod, seed, or progeny of the plant of claim 1.

9. A mutant allele of a Brassica CKX3 or CKX5 gene, wherein the CKX5 gene is:
(a) a nucleotide sequence which comprises at least 90% sequence identity to SEQ ID NO: 19 or SEQ ID NO: 23;
(b) a nucleotide sequence comprising a coding sequence which comprises at least 90% sequence identity to SEQ ID NO: 20 or SEQ ID NO: 23; or
(c) a nucleotide sequence encoding an amino acid sequence which comprises at least 90% sequence identity to SEQ ID NO: 21, or SEQ ID NO: 24; and
wherein the CKX3 gene is
(d) a nucleotide sequence which comprises at least 90% sequence identity to SEQ ID NO: 7, SEQ ID NO: 10; SEQ ID NO: 13 or SEQ ID NO: 16;
(e) a nucleotide sequence comprising a coding sequence which comprises at least 90% sequence identity to SEQ ID NO: 8, SEQ ID NO: 11; SEQ ID NO: 14 or SEQ ID NO: 17; or
(f) a nucleotide sequence encoding an amino acid sequence which comprises at least 90% sequence identity to SEQ ID NO: 9, SEQ ID NO: 12; SEQ ID NO: 15 or SEQ ID NO: 18.

10. The mutant allele according to claim 9, wherein said mutant allele is:
a. a mutant CKX5 allele comprising a G to A substitution at a position corresponding to position 465 of SEQ ID NO: 19 or position 465 of SEQ ID No. 20;
b. a mutant CKX5 allele comprising a G to A substitution at a position corresponding to position 399 of SEQ ID NO: 19 or position 399 of SEQ ID No. 20; and
c. a mutant CKX5 allele comprising a G to A substitution at a position corresponding to position 465 of SEQ ID NO: 22 or position 399 of SEQ ID No. 23;
d. a mutant CKX3 allele comprising a C to T substitution at a position corresponding to position 2244 of SEQ ID NO: 7 or position 1093 of SEQ ID No. 8;
e. a mutant CKX3 allele comprising a C to T substitution at a position corresponding to position 2482 of SEQ ID NO: 10 or position 1168 of SEQ ID No. 11;
f. a mutant CKX3 allele comprising a G to A substitution at a position corresponding to position 1893 of SEQ ID NO: 13 or position 876 of SEQ ID No. 14; or
g. a mutant CKX3 allele comprising a C to T substitution at a position corresponding to position 2171 of SEQ ID NO: 16 or position 982 of SEQ ID No. 17.

11. A method to increase flower number per plant, to increase Thousand Seed Weight or to increase pod number per plant comprising introducing at least one mutant CKX5 allele or at least one mutant CKX5 allele and one mutant CKX3 allele into a Brassica plant.

12. A Brassica plant:
comprising a mutant CKX5 allele comprising a G to A substitution at a position corresponding to position 465 of SEQ ID NO: 19 or position 465 of SEQ ID No. 20, reference seeds comprising said allele having been deposited at the NCIMB Limited, under accession number NCIMB 42464;
comprising a mutant CKX5 allele comprising a G to A substitution at a position corresponding to position 399 of SEQ ID NO: 19 or position 399 of SEQ ID No. 20 reference seeds comprising said allele having been deposited at the NCIMB Limited, under accession number NCIMB 42465;
comprising a mutant CKX5 allele comprising a G to A substitution at a position corresponding to position 465 of SEQ ID NO: 22 or position 399 of SEQ ID No. 23, reference seeds comprising said allele having been deposited at the NCIMB Limited, under accession number NCIMB 42464;
comprising a mutant CKX3 allele comprising a C to T substitution at a position corresponding to position 2244 of SEQ ID NO: 7 or position 1093 of SEQ ID No. 8, reference seeds comprising said allele having been deposited at the NCIMB Limited, under accession number NCIMB 42464;
comprising a mutant CKX3 allele comprising a C to T substitution at a position corresponding to position 2482 of SEQ ID NO: 10 or position 1168 of SEQ ID No. 11, reference seeds comprising said allele having been deposited at the NCIMB Limited, under accession number NCIMB 42464;
comprising a mutant CKX3 allele comprising a G to A substitution at a position corresponding to position 1893 of SEQ ID NO: 13 or position 876 of SEQ ID No. 14, reference seeds comprising said allele having been deposited at the NCIMB Limited, under accession number NCIMB 42464; or
comprising a mutant CKX3 allele comprising a C to T substitution at a position corresponding to position 2171 of SEQ ID NO: 16 or position 982 of SEQ ID No. 17, reference seeds comprising said allele having been deposited at the NCIMB Limited, under accession number NCIMB 42464.

13. A method to increase flower number per plant, pod number per plant or increase TSW in Brassica plants comprising introducing at least one mutant allele of claim 9 into a Brassica plant.

14. A method for producing oilseed rape oil or an oilseed rape seed cake from the plant of claim 1.

15. A method for producing food, feed, or an industrial product comprising
preparing food, feed or an industrial product from the plant or part thereof of claim 1.
